(19) United States
(12) Patent Application Publication
(10) Pub. No.: US 2017/0345332 A1
(43) Pub. Date: Nov. 30, 2017

(54) METHOD OF AND SYSTEM FOR FACILITATING OPERATION OF A HEALTH MAINTENANCE DEVICE

(71) Applicant: Rodney Obay, Portland, OR (US)

(72) Inventor: Rodney Obay, Portland, OR (US)

(21) Appl. No.: 15/602,937

(22) Filed: May 23, 2017

Related U.S. Application Data
(60) Provisional application No. 62/340,667, filed on May 24, 2016.

(51) Int. Cl. G09B 19/00; A61B 5/00; G06F 19/00; A61B 5/11; A63B 24/00

(52) U.S. Cl. CPC G09B 19/00; A61B 5/11; A63B 24/0062; G09B 19/0092; G06F 19/3406; A61B 5/0002; A63B 2024/0065; A63B 2024/0068; A63B 2220/30; A63B 2225/50; A63B 2230/01; A63B 2220/20

(57) ABSTRACT

Disclosed is a method of facilitating operation of a health maintenance device. The method may include receiving a plurality of health maintenance participant information associated with a user. At least a part of the plurality of health maintenance participant information may be generated based on monitoring each of the health maintenance device and the user. Further, the method may include generating, using a processing device, a plurality of performance assessments of the user based on the plurality of the health maintenance participant information. Furthermore, the method may include generating at least one control code based on a comparing of the plurality of performance assessments with a health maintenance database. Additionally, the method may include transmitting the at least one control code to the health maintenance device. Further, the health maintenance device may be configured to be adjusted based on the at least one control code.

The current application claims a priority to the U.S. Provisional Patent application Ser. No. 62/340,667 filed on May 24, 2016.

### FIELD OF DISCLOSURE

The present disclosure generally relates to health maintenance devices. More specifically, the disclosure relates to a method of and system for facilitating operation of a health maintenance device, such as, but not limited to, a fitness maintenance device, a nutrition maintenance device, etc.

### BACKGROUND

An epidemic of obesity continues to sweep across the adult and adolescent landscape in America today. Even with advancements in the sports medicine and exercise science industry, our overweight and obese citizens remain both underserved and underappreciated.

In some parts of America, the high body mass and makeup of overweight and obese lives are complicated with the addition of medical and malnutrition issues. To date, there appears to be no globally accepted fitness and nutrition (i.e. fitness maintenance) system that monitors, tracks and assists individuals, such as overweight and obese populations.

Furthermore, existing fitness maintenance measurement systems that attempt to improve the overweight and obese citizens lack a nutrition periodization measurement system that adjusts to populations of high body mass and makeup percentages of adolescents and adults. Further, such fitness maintenance measurement systems fail to consider individual, geographical and cultural restrictions. As a result, it potentially leads to poor or improper nutrition periodization and dietary consumption patterns.

Further, overweight and obese citizens also suffer from the lack of a comprehensive and coordinated approach that would change their environmental factors to include access to full-service grocery stores with reasonably priced healthy foods and safe places to play and exercise. Accordingly, individuals and groups with high body mass index percentages have inherent social and economic obstacles to meaningful fitness, nutrition and overall health products and services.

Accordingly, there is a need for methods and systems for facilitating maintenance of health among users while overcoming one or more of the aforementioned drawbacks.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

A computer implemented method of facilitating operation of a health maintenance device is provided. The computer implemented method may include receiving, using a communication device, a first plurality of health maintenance participant information associated with a health maintenance user. Further, the first plurality of health maintenance participant information may include a selection of one or more of an interactive learned program mode and an automated health maintenance test mode. Furthermore, the computer implemented method may include receiving, using a communication device, a second plurality of health maintenance participant information generated based on monitoring each of the health maintenance device and the health maintenance user. Additionally, the computer implemented method may include generating, using a processing device, a plurality of performance assessments of the health maintenance user corresponding to a plurality of health maintenance sessions of the health maintenance device. Further, each performance assessment may be generated based on the second plurality of the health maintenance participant information and the first plurality of health maintenance participant information. Furthermore, the computer implemented method may include storing, using a storage device, the plurality of performance assessments. Additionally, the computer implemented method may include generating, using the processing device, at least one control code based on a comparing of the second plurality of health maintenance participant information with the health maintenance database. Further, the computer implemented method may include transmitting, using the communication device, the at least one control code to the health maintenance device. Furthermore, the health maintenance device may be configured to be adjusted based on the at least one control code.

In some embodiments, the first plurality of health maintenance information may include one or more of user data, at least one goal and a selection of an operation mode of the health maintenance device.

In some embodiments, a performance assessment of the plurality of performance assessments may include an energy expenditure associated with the at least one goal.

In some embodiments, the performance assessment further may include a percentage of reaching the at least one goal. Further, the percentage may be determined based on dividing the energy expenditure by one or more of a weight and a time length of use of the health maintenance device.

In some embodiments, the computer implemented method may further include: generating, using the processing device, a suggested goal based on a comparison of the percentage with a predetermined low; and transmitting, using the communication device, the suggested goal to the health maintenance device.

In some embodiments, the at least one control code may be configured to adjust one or more of a difficulty level and an environmental factor associated with the health maintenance device.

In some embodiments, generating the at least one control code may be further based on the first plurality of health maintenance participant information.

In some embodiments, the second plurality of health maintenance participant information may include one or more of a weight, a speed and a distance traveled associated with the health maintenance device. Further, the health maintenance device may include at least one sensor configured to detect one or more of the weight, the speed and the distance travelled.

In some embodiments, the computer implemented method may further include: generating, using the processing device, at least one predetermined health maintenance boundary associated with the health maintenance device. Further, the at least one predetermined health maintenance boundary may be generated based on the user data; and transmitting, using the communication device, the at least one predetermined health maintenance boundary to the health maintenance device. Further, the health maintenance device may be configured to generate an alert based on an operational value of the health maintenance device exceeding the at least one predetermined health maintenance boundary.

In some embodiments, the user data may include a physical condition. Further, the predetermined health maintenance boundary may be further associated with physiological data of the health maintenance user. Further, the health maintenance device may be further configured to detect physiological data of the health maintenance user.

Also, a system for facilitating operation of a health maintenance device is provided. The system may include a communication device configured to receive a first plurality of health maintenance participant information associated with a health maintenance user. Further, the first plurality of health maintenance participant information may include a selection of one or more of an interactive learned program mode and an automated health maintenance test mode. Furthermore, the communication device may be configured to receive a second plurality of health maintenance participant information generated based on monitoring each of the health maintenance device and the health maintenance user. Additionally, the communication device may be configured to transmit at least one control code to the health maintenance device. Further, the health maintenance device may be configured to be adjusted based on the at least one control code. Moreover, the system may include a processing device configured to generate a plurality of performance assessments of the health maintenance user corresponding to a plurality of health maintenance sessions of the health maintenance device. Further, each performance assessment may be generated based on the second plurality of the health maintenance participant information and the first plurality of health maintenance participant information. Additionally, the processing device may be configured to generate the at least one control code based on a comparing of the second plurality of health maintenance participant information with the health maintenance database. Further, the system may include a storage device configured to store the plurality of performance assessments.

In some embodiments, the first plurality of health maintenance information may include one or more of user data, at least one goal and a selection of an operation mode of the health maintenance device.

In some embodiments, a performance assessment of the plurality of performance assessments may include an energy expenditure associated with the at least one goal.

In some embodiments, the performance assessment further may include a percentage of reaching the at least one goal. Further, the percentage may be determined based on dividing the energy expenditure by one or more of a weight and a time length of use of the health maintenance device.

In some embodiments, the processing device may be further configured to generate a suggested goal based on a comparison of the percentage with a predetermined low. Further, the communication device may be further configured to transmit the suggested goal to the health maintenance device.

In some embodiments, the at least one control code may be configured to adjust one or more of a difficulty level and an environmental factor associated with the health maintenance device.

In some embodiments, generating the at least one control code may be further based on the first plurality of health maintenance participant information.

In some embodiments, the second plurality of health maintenance participant information may include one or more of a weight, a speed and a distance traveled associated with the health maintenance device. Further, the health maintenance device may include at least one sensor configured to detect one or more of the weight, the speed and the distance travelled.

In some embodiments, the processing device may be further configured to generate at least one predetermined health maintenance boundary associated with the health maintenance device. Further, the at least one predetermined health maintenance boundary may be generated based on the user data. Further, the communication device may be further configured to transmit the at least one predetermined health maintenance boundary to the health maintenance device. Further, the health maintenance device may be configured to generate an alert based on an operational value of the health maintenance device exceeding the at least one predetermined health maintenance boundary.

In some embodiments, the user data may include a physical condition. Further, the predetermined health maintenance boundary may be further associated with physiological data of the health maintenance user. Further, the health maintenance device may be further configured to detect physiological data of the health maintenance user.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 illustrates a block diagram of an operating environment consistent with the present disclosure.

FIG. 2 illustrates a system for facilitating operation of a health maintenance device, in accordance with some embodiments.

FIG. 3 illustrates a system for facilitating operation of a plurality of health maintenance devices, in accordance with some embodiments.

FIG. 4 illustrates a method of facilitating operation of a health maintenance device, in accordance with some embodiments.

FIG. 5 illustrates a method of facilitating operation of a health maintenance device by suggesting a health maintenance goal, in accordance with some embodiments.

FIG. 6 illustrates a method of facilitating operation of a health maintenance device based on user data (such as, but not limited to, clinical considerations) associated with a user, in accordance with some embodiments.

FIG. 7 illustrates a block diagram of a system for facilitating serving of an order at a facility, in accordance with some embodiment.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of health maintenance, embodiments of the present disclosure are not limited to use only in this context.

I. Platform Overview

Consistent with embodiments of the present disclosure, an online platform (also referred to herein as “platform” or health maintenance platform or O'Bay System) for facilitating maintenance of health of individuals may be provided. This overview is provided to introduce a selection of concepts in a simplified form that are further described below. This overview is not intended to identify key features or essential features of the claimed subject matter. Nor is this overview intended to be used to limit the claimed subject matter's scope. The online platform may be used by individuals or companies to facilitate management of health maintenance of users and operation of health maintenance devices.

In some embodiments, the online platform may be configured to prevent, eliminate or significantly reduce obesity by promoting and providing onsite and mobile fitness maintenance testing, analysis and training applications and technologies.

The online platform may be designed as an onsite and mobile fitness maintenance testing, analysis and training system and infrastructure. This infrastructure may use the latest video, computer science, information and artificial intelligence technology for practical onsite and mobile fitness maintenance applications for high and low body mass index individuals, groups and communities.

All high and low body mass index individuals and groups' criteria may be monitored and tracked over time and/or distance and/or calorie consumption and/or weight, for improvements to their personal wellness through fitness maintenance conditioning. Further, the online platform may be configured to disseminate meaningful fitness maintenance information when dealing with the complex nature of high body mass index individuals and groups.

Further, the online platform may enable extensive documentation of the fitness maintenance values, in coordination with individual and group consumption pattern. Additionally, the online platform may be configured to define and explain the strengths and weaknesses of its onsite and mobile fitness maintenance system when applied daily. By gathering and using onsite and mobile fitness maintenance information, the online platform may provide meaningful benchmarks for triathlon and nutrition maintenance participants with high body mass or makeup, irrespective of their fitness level, age and gender.

The online platform may also establish and administer a uniquely, individualized nutrition metric system using the consumption of therapeutic amounts macronutrient-based blood-type specific, kosher and organic foods and drink under the Nationally Recommended Dietary Allowance standard and scale of the country of operation. This World Nutrition Metric System may be added to the dietary rotation of high and low body mass index participants with the incrementally elimination or avoidance of manmade or genetically engineered foods and drinks. All high and low body mass index participants, irrespective of their fitness maintenance status, may be encouraged to adopt this dietary macronutrient-based consumption rotation.

To document and validate the short-term, medium and long-term effects and benefits of using the online platform, the online platform may also establish its World Fitness Maintenance Metric Standard and Scale through its policies and procedures.

Further, online platform may be configured to develop and establish the World Fitness Maintenance Metric Standard and Scale. The triathlon and nutrition metric standard and scale may monitor, dispense and record high and low body mass index triathlon and nutrition maintenance performances through the routine evaluation of sports medicine and exercise science laboratory information. The triathlon and nutrition metric standard and scale may also integrate and incorporate this information to document, predict and improve sports medicine and exercise information application to all high body mass index age groups ordinarily excluded by either personal, social or economic barriers.

The online platform may employ the latest sports medicine and exercise science technology during triathlon and nutrition maintenance performance activities, over time and/or distance and/or calorie consumption and/or weight, to document changes or improvements to individual and group high and low body mass index performances. Through analyses, education, counseling, monitoring, evaluation, simulation and recordings, the online platform may gather and apply its triathlon and nutrition maintenance system to selected population to document, prevent and improve the triathlon and nutrition maintenance status of those high and low body mass index individuals and groups while in a study capacity.

The online platform may be configured to create uniform, triathlon and nutrition maintenance standards and scales for high and low body mass index individual, group and participants who struggle with an increased body mass and makeup.

The online platform may also monitor underweight fitness participants during individual and group triathlon and nutrition maintenance performances based upon variables like age, body composition or mass concerns when applied proportionally. For triathlon and nutrition maintenance regiments, to provide meaningful fitness baselines for underweight populations, it may also be applied over various weight-related fitness, nutrition and health categories.

By using various fitness participants from weight and age-specific control groups, the online platform may compare individual and group factors while properly defining triathlon and nutrition maintenance performance through guidelines and information uniformity. The online platform may develop an invaluable, standardized form of sports medicine and exercise information for the all citizens of the world.

The online platform may include, but not limited to VO2 max test, lactate threshold analysis, metabolic rate analysis, swim-stroke force analysis, dry-land strength test, spin scan, motion analysis and body composition.

The online platform may use any computer sensory units to monitor and record the degrees of the fitness and nutrition periodization performance by the participants under our triathlon and nutrition maintenance regiment. The online platform also use the latest form of communication devices available, including personal, online and wireless computer, to create and monitor high and low body mass index individual and group participation.

Accordingly, the online platform may develop and document its fitness maintenance applications as a method for high and low body mass index individual or group based upon properly kept research, data collection and analysis.

As a result, the online platform may help to predict and prevent future trends of overweight and obese individuals or groups while establishing the parameters of the World Fitness Maintenance Metric Standard and Scale incorporated herein.

In some embodiments, the O'Bay Fitness Maintenance System (also referred to herein as the O'Bay System) may be a secured fitness and/or nutrition application system and method for review and analyses of the fitness and/or nutrition participant by one or more health and fitness and/or nutrition options between the sports medicine and exercise physiologist M.D. or Ph.D. and the exercise physiologist and dietician/nutritionist or certified personal trainer located at the onsite or mobile sports medicine and exercise science site. Under the care of the sports medicine or exercise physiologist M.D. or Ph.D., located at a sports medicine or exercise physiologist M.D. or Ph.D. site physically separated from the onsite or mobile sports medicine and exercise science site, the interactive computer assisted fitness and/or nutrition method or an artificial intelligence system may provide guidance and assistance to the fitness and/or nutrition personal in the fitness and/or nutrition environment.

In some embodiments, the O'Bay System may include a fitness and/or nutrition surface interface system that may be a display device associated with the fitness and/or nutrition device that includes connecting the fitness and/or nutrition network connection to the exercise device capable of accessing information stored over the network, partitioning a first portion of the display device associated with the fitness and/or nutrition device to display content, partitioning a second portion of the display device associated with the fitness and/or nutrition surface to display one or more controls that control the fitness and/or nutrition device, and providing a selectable element on the display device that upon selection removes the second portion of the display used to display the one or more controls.

In some embodiments, the O'Bay System includes a fitness and/or nutrition system that consists of tailor-made fitness and/or nutrition protocol that can be modified by sports medicine and exercise science clinician in a supervisory position. A processor or an artificial intelligence system with a protocol-generating algorithm may be used in communication with the fitness and/or nutrition device. Sports medicine and exercise science information data may be input into the processor or an artificial intelligence system, such as age, height, sensor and sex, and the processor or an artificial intelligence system generate the fitness and/or nutrition protocol per a protocol-generating algorithm of the sports medicine and exercise science data. A fitness and/or nutrition supervisor may be then capable of reviewing and allowing for modification of the fitness and/or nutrition protocol. After the fitness and/or nutrition protocol may be approved, it may be transferred to the fitness and/or nutrition device and surface. The fitness and/or nutrition participants can then perform the fitness and/or nutrition session on the fitness and/or nutrition surface. Sensors on the fitness and/or nutrition device of the exercise physiologist and dietician/nutritionist or certified personal trainer generate information regarding the fitness and/or nutrition session. This sports medicine and exercise science information may be transferred back to the processor or an artificial intelligence system where it may be reviewed by the fitness and/or nutrition supervisor where the fitness and/or nutrition supervisor can modify the sports medicine and exercise science protocol for the next fitness and/or nutrition session if they deem it necessary.

In some embodiments, the O'Bay System may also include a virtual reality fitness chamber. This virtual reality fitness chamber comprises a virtual reality fitness simulator system using integrated virtual reality fitness components that accurately quantifies and qualifies the virtual reality fitness experience through individual and team virtual reality fitness performance constructs by employing sensing electronics for determining, in essentially real time, the participant's three dimensional positional changes in three or more degrees of freedom (three dimensions). The nationally certified exercise physiologist and dietician/nutritionist or computer controlled, cuing that evokes or prompts virtual reality fitness responses from the participant that are measured to provide meaningful indicia of the virtual reality fitness performance. The virtual reality fitness performance cuing may be characterized as a virtual reality fitness participant that may be responsive to and interactive with the virtual reality fitness participant in real time. A three dimensional, virtual or interactive nationally certified exercise physiologist and dietician/nutritionist may continually deliver or responds to stimuli to create realistic movement challenges for the virtual reality fitness participant.

In some embodiments, the O'Bay System also include a triathlon and nutrition periodization application system. The triathlon and nutrition periodization application system comprises a secured, peer-to-peer, triathlon and nutrition periodization information application for devices that are communicable with a supervisory computer through remote triathlon and nutrition periodization sites; the triathlon and nutrition periodization participants are matched with the triathlon and nutrition periodization skill level may be manually operable by the triathlon and nutrition periodization user for generating the triathlon and nutrition periodization performance. The monitor may display the triathlon and nutrition periodization measurements performance test and training score that match with the triathlon and nutrition periodization level so that the triathlon and nutrition periodization measurements user may be prompted to render the displayed triathlon and nutrition periodization measurements performance, lesson and score by operating the triathlon and nutrition periodization measurements peer-to-peer application and device for generating a sample triathlon and nutrition periodization measurements performance. the triathlon and nutrition periodization measurements processor or an artificial intelligence system compares triathlon and nutrition periodization measurements event data representative of the sample triathlon and nutrition periodization measurements performance with note triathlon and nutrition periodization measurements data representative of the triathlon and nutrition periodization measurements lesson and score to locally execute a quantitative and qualitative evaluation of the sample triathlon and nutrition periodization measurements performance. the triathlon and nutrition periodization transmitter transmits the triathlon and nutrition periodization event data representative of the sample triathlon and nutrition periodization performance to the supervisory computer through the World Fitness Maintenance Network so that the supervisory computer can work to remotely provide a qualitative triathlon and nutrition periodization performance evaluation of the sample triathlon and nutrition periodization performance according to the triathlon and nutrition periodization event data to thereby arrange triathlon and nutrition periodization instruction data.

In some embodiments, the O'Bay System may also comprise the World Nutrition Metric System™. The World Nutrition Metric System may be the incremental, oral administration and nutrition consumption of therapeutic amounts of macronutrients-based, blood-type specific kosher and organic foods and drink under the Nationally Recommended Dietary Allowance standard and scale of the country of operation (i.e. in America, the Dietary Guidelines for Americans). Along with periodical fasting from individually toxic food items, this macronutrients-based nutrition metric system may systematically and methodically eliminate man-made, genetically-engineered or altered food products for the participant's food and drink nutrition consumption rotation. The World Nutrition Metric System may also provide an individualized, macronutrients-based consumption plan of blood-type specific kosher and organic foods and drinks that support the participant through various levels of their triathlon and nutrition and educational counseling, one-on-one, and group, computer-based or video instruction within our fitness facility. The applying macronutrients-based nutrition standards may be known as the World Nutrition Metric Allowance Standard and Scale™. The World Nutrition Metric System may also be known as Nutrimetrics Program or System™.

In some embodiments, the O'Bay System may include the World Fitness Maintenance Metric Standard and Scale™ (O'Baymetrics™). The World Fitness Maintenance Metric Standard and Scale consists of the scientific quantification, interpretation, correlation and tracking method and system of the peak, health, wellness, overweight and obesity fitness maintenance metric quotient score, along with the fitness maintenance metric risks, resulting with output reports and summary of the graduated ranking numerical metric known as the World Fitness Maintenance Metric Scale and Factoring Formula. Data gathering forms, devices, documents, sources and questionnaires may be divided into different categories of peak, health, wellness, overweight and obesity fitness maintenance metric quotient score, along with the fitness maintenance metric risks which affect fitness maintenance metric participants arranged so that the data gathered are in the form of graduated numerical scores against a specified fitness maintenance metric ranking scale. The analysis, quantification, interpretation, correlation, ranking, tracking, over time and/or distance and/or calorie consumption and/or weight, and appropriate suggestions are presented in computer printed form for the respective categories in a manner comparing ideal fitness maintenance metric quotient scores and ultra-low-risk conditions against actual conditions of the affect fitness maintenance metric participants. measurements may be taken by certified fitness trainers and technicians, whose evaluations and suggestions, selected from a data bank per score, may be graduated and correlated to the scores on the 10-point fitness maintenance metric standard. The output report may be a computer printed, color-accented, numerically comparative graphic showing how peak, health, wellness, overweight and obesity fitness maintenance metric quotient score may be achieved and the fitness maintenance metric risk rating of the fitness maintenance metric participant in their respective categories. Comparison within the peak, health, wellness, overweight and obesity fitness maintenance metric quotient score categories, respectively, may rate and rank fitness maintenance metric participant levels identified as excellent, good, average, poor, or dangerous. The 10-Point World Fitness Maintenance Metric Standard™, which are cross-correlated to the actual and weighted scores on, may be adjusted by factoring based upon the peak, health, wellness and obesity fitness maintenance metric quotient score categories. The development and implementation of a unique, proprietary algorithm for overall World Fitness Maintenance Metric insurability known as the O'Baymetrics Quotient (OQ™) record, a personal O'Baymetrics Quotient or Score, that should be, accompanied by a financial (insurance) incentive feature wherein premiums and deductibles can be applied.

The World Fitness Maintenance Metric Standard and Scale™ may also consist of, but not limited to, assessing, measuring, quantifying, evaluating, validating, correlating, comparing, interpreting, ranking and tracking, over time and/or distance and/or calorie consumption and/or weight, incremental changes in the total World Fitness Maintenance Metric. The O'Baymetrics Quotient score may include information like VO2 max test, lactate threshold analysis, metabolic rate analysis, swim-stroke force analysis, dry-land strength test, spin scan, motion analysis, body composition al measurements before and after selected fitness maintenance metric quotient score performances. The World Fitness Maintenance Metric Standard and Scale may also generate O'Baymetrics Quotient score analysis, testing, research, documentation and tracking through laboratory analyzed levels of total Fitness Maintenance Metric participant optimal health and overall associated with health, Fitness Maintenance Metric risks through predicting, improving and forecasting individual and group Fitness Maintenance Metric gains and losses.

In the nutrition periodization stage of World Fitness Maintenance Metric System, the participants may be encouraged to use the World Nutrition Metric System, i.e. the therapeutic amounts of macronutrient-based blood-type specific kosher and organic food and drink, along with a fitness maintenance metric regiment. Specific high and low risk health individuals and groups, like diabetics, may be studied for fitness maintenance metric quotient score analysis, testing, research, documentation, tracking and research through laboratory analyzed levels of total and nutrition periodization metrics, optimal health and overall associated fitness maintenance metric risks to predict, improve and forecast future individual and societal gains and losses.

In some embodiments, the O'Bay System also comprises an individualized, onsite and mobile peak fitness and/or nutrition periodization program, training, education, counseling, one-on-one, group, computer-based or video instruction means to peak fitness and/or nutrition periodization participants within our fitness maintenance facility.

This program may generate an Individualized Fitness Maintenance Plan for peak fitness and/or nutrition periodization training participants.

This may be administered throughout all stages of the Individualized Fitness Plan™ (IFP™) and Individualized Nutrition plan (INP™) evaluation, education and training stages of the research institution within a fitness maintenance facility.

By creating and administering Individualized Fitness Plan (IFP™) and Individualized Nutrition Plan (INP™), the program may provide the peak fitness and/or nutrition periodization training means to the peak fitness and/or nutrition periodization training participants interested in their peak fitness and/or nutrition periodization training endeavor within the fitness maintenance facility.

In some embodiments, a computer software program of the World Fitness Maintenance Network may be provided. This program enables research institution employees and supervisors to track systemic entries. The entry on the World Fitness Maintenance Network may create an O'Baymetrics Quotient Record (OQR) based on the participant's profile, tracking, over time and/or distance and/or calorie consumption and/or weight, the progress and performances during periodic evaluations and assessments.

Along with an adolescent entry qualification evaluations and parental authorization, a Peak Fitness Research Record (PFR2) may be generated with any peak fitness maintenance training entry form within the World Fitness Maintenance Network.

An adolescent Individualized Fitness Plan, Individualized Nutrition Plan and O'Baymetrics Quotient Record (OQR) may be automated and integrated into the World Fitness Maintenance Network of the Research institution.

An O'Baymetrics Quotient Research Record (OQR2) may also be generated when the peak fitness maintenance training participant plans meet all legal requirements for administering health, triathlon and nutrition maintenance research.

The O'Baymetrics Quotient Research Record (OQR2) must meet any legal requirements of a local, state or Federal Department of Justice, Health and Human Services standards of a given country when necessary for the completion and submission of peak fitness maintenance training participant's training form.

Records may not be added to the personal or professional records of a peak fitness and/or nutrition periodization training participant without the administrative authorization from either the medical or exercise physiologist and dietician/nutritionist director of the Research institution.

In some embodiments, the O'Bay System also comprise a fitness maintenance center program with an individualized, onsite and mobile fitness and/or nutrition program, training, education, counseling, one-on-one, group, computer-based or video instruction means to medically and weight-challenged fitness and/or nutrition participants within the research institution.

This program may generate Individualized Fitness Plan (IFP™), Individualized Triathlon Plan (ITP™) and Individualized Nutrition plan (INP™) for all medical and weight-challenged fitness and/or nutrition participants within the research institution.

This program may be administered throughout all program stages of the Individualized Fitness Plan™ (IFP™), Individualized Triathlon Plan (ITP™) and Individualized Nutrition Plan™ (INP™) evaluation, education and training stages through the research institution within the fitness maintenance facility.

By creating and administering an Individualized Fitness Plan™ (IFP™), Individualized Triathlon Plan™ (ITP™) and Individualized Nutrition Plan™ (INP™), the program may provide the triathlon, fitness and/or nutrition means to the medically and weight-challenged fitness and/or nutrition participants interested in their fitness endeavor.

Embodied in the computer software program of the World Fitness Maintenance Network, the program enables the research institution employees and supervisors to track systemic entries. The entry on the World Fitness Maintenance Network may create the World Fitness Maintenance Network record based on the participant's profile, tracking, over time and/or distance and/or calorie consumption and/or weight, the progress and performs during periodic evaluations and assessments.

Along with an adult or adolescent entry qualification evaluations and parental authorization, an O'Baymetrics Quotient Record™ (OQR™) and an O'Baymetrics Quotient Research Record™ (OQR2™) may be generated with any triathlon, fitness and/or nutrition training entry form within the World Fitness Maintenance Network of the research institution.

An adolescent or adult Individualized Fitness Plan (IFP™), Individualized Triathlon Plan™ (ITP™), Individualized Nutrition Plan (INP™) and an O'Baymetrics Quotient Record™ (OQR™) may be automated and integrated into the fitness maintenance database of the research institution as an O'Baymetrics Quotient Research Record™ (OQR2™) when the medically and weight-challenged fitness participant plans meet all legal requirements for administering health, fitness maintenance metric research.

The O'Baymetrics Quotient Research Record™ (OQR2™) must meet any legal requirements of a local, state or Federal Department of Justice, Health and Human Services standards of a given country when necessary for the completion and submission of a medically and weight-challenged triathlon participant's training form.

Records may not be added to the personal or professional records of a medically and weight-challenged triathlon participant without the administrative authorization of the medical or exercise physiologist and dietician/nutritionist director.

In some embodiments, the O'Bay System may also comprise the research institution program. The research institution program may provide and monitor all individualized onsite and mobile triathlon, fitness, cycling fitness maintenance testing, analysis, training, research, education, counseling, one-on-one, group, computer-based or video instruction means to fitness research participants within our fitness facility.

The institute program may provide and monitor all Individualized Fitness Plan (IFP™), Individualized Triathlon Plan (ITP™), Individualized Cycling Fitness Maintenance Plan (ICP™) and Individualized Nutrition plan (INP™) within our fitness maintenance facility.

By creating and administering all Individualized Fitness Plan (IFP™), Individualized Triathlon Plan (ITP™), Individualized Cycling Fitness Maintenance Plan (ICP™) and Individualized Nutrition plan (INP™) within our fitness facility, the research institution program may be able to monitor and record the statistical fitness and/or nutrition periodization means and results of fitness participants before, during and after their fitness endeavor.

Embodied in the computer software program of the World Fitness Maintenance Network, the Research institution program may enable the research institution supervisors and employees to track systemic entries for research purposes.

The entry on the World Fitness Maintenance Network may create an O'Baymetrics Quotient Research Record based on the adult participant's research profile, tracking, over time and/or distance and/or calorie consumption and/or weight, the progress and perform during periodic fitness and/or nutrition periodization evaluations and assessments.

Along with an adult or adolescent entry qualification evaluations and parental authorization, an O'Baymetrics Quotient Research Record may be generated with any triathlon and nutrition metric entry form within the World Fitness Maintenance Network of the research institution.

An Individualized Fitness Plan (IFP™), Individualized Triathlon Plan (ITP™), Individualized Cycling Fitness Maintenance Plan (ICP™), Individualized Fitness Plan™ (IFP™) and Individualized Nutrition Plan (INP™) and O'Baymetrics Quotient Record™ (OQR™) may be automated and integrated into the World Fitness Maintenance Network of the research institution as an O'Baymetrics Quotient Research Record (OQR2) when the fitness participant plans meet all legal requirements for administering health and fitness research.

All O'Baymetrics Quotient Research Record (OQR2) must meet any legal and research requirements of a local, state or Federal Department of Justice, Health and Human Services standards of a given country when necessary for the completion and submission of a triathlon participant's research form.

All records may not be added to the personal or professional records of a triathlon participant without the administrative authorization of the medical director for the Research institution.

The O'Bay System may include the Triathlon and Nutrition Periodization Training System. The Triathlon and Nutrition Periodization Training System™ may be the triathlon and nutrition periodization trainee system and method for training and certifying triathlon and nutrition periodization trainees to perform triathlon and nutrition periodization tasks. The system includes an input device, output device and a controller. The controller receives input data from the input device and controls the output displayed on the output device.

In some embodiments, the O'Bay System may include the Triathlon Training Adolescent Program. The Triathlon Training Adolescent Program may provide an individualized, onsite or mobile triathlon and nutrition training education, counseling, one-on-one, group, computer-based or video instruction means to adolescent fitness participants within our fitness facility.

The Triathlon Training Adolescent Program may generate an adolescent Individualized Triathlon Plan (ITP™) through the onsite and mobile sports medicine and exercise science services of the Research institution within our fitness facility.

The Triathlon Training Adolescent Program may be administered throughout all stages of the Individualized Triathlon Plan™ (ITP™) evaluation, education and training stages through the Research institution.

By creating and administering an adolescent Individualized Triathlon Plan (ITP™), the Triathlon Training Adolescent Program may provide the triathlon and nutrition means to the adolescent participants interested in their fitness endeavor.

Embodied in the computer software program of the World Fitness Maintenance Network, the TPAP program enables Research institution employees and supervisors to track systemic entries. The entry on the World Fitness Maintenance Network may create an O'Baymetrics Quotient™ (OQ™) based on the participant's profile, tracking, over time and/or distance and/or calorie consumption and/or weight, the progress and perform during periodic triathlon and nutrition metric evaluations and assessments.

Along with an adolescent entry qualification evaluations and parental authorization, an O'Baymetrics Quotient Research Record™ (OQR2™) may be generated with any obesity-specific fitness entry within the World Fitness Maintenance Network.

An adolescent Individualized Triathlon Plan (ITP™) and an O'Baymetrics Quotient Record™ (OQR™) may be automated and integrated into the World Fitness Maintenance Network of the Research institution as an O'Baymetrics Quotient Research Record™ (OQR2™) when the adolescent participant plans meet all legal requirements for administering health and fitness maintenance research.

An O'Baymetrics Quotient Research Record™ (OQR2™) must meet any legal requirements of a local, state or Federal Department of Justice, Health and Human Services standards of a given country when necessary for the completion and submission of the adolescent participant's training form.

Records may not be added to the personal or professional records of the adolescent participant without the administrative authorization from either the medical or exercise physiologist and dietician/nutritionist director.

In some embodiments, the O'Bay System may include the Triathlon Training Individual Program. The Triathlon Training Individual Program may provide Individualized, onsite and mobile triathlon and nutrition education, counseling, one-on-one, group, computer-based or video instruction means to an adult participant within our fitness maintenance facility.

The Triathlon Training Individual Program may generate an adult, Individualized Triathlon Plan (ITP™) through the onsite and mobile sports medicine and exercise science services through the Health and Fitness Research institution.

The Triathlon Training Individual Program may be administered throughout all stages of the Individualized Triathlon Plan™ (ITP™) evaluation, education and training stages of the Research institution within our fitness facility.

By creating and administering Individualized Triathlon Plan (ITP™), the Triathlon Training Individual Program may provide the triathlon and nutrition training means to the triathlon participants interested in their competitive-driven triathlon endeavor.

Embodied in the computer software program of the World Fitness Maintenance Network, the Triathlon Training Individual Program enables research institution employees and supervisors to track systemic entries. The entry on the World Fitness Maintenance Network may create an O'Baymetrics Quotient Record™ (OQR™) based on the participant's profile, tracking, over time and/or distance and/or calorie consumption and/or weight, the progress and perform during periodic evaluations and assessments.

An adult Individualized Triathlon Plan (ITP™) and an O'Baymetrics Quotient Record™ (OQR™) may be automated and integrated into the World Fitness Maintenance Network of the Research institution as an O'Baymetrics Quotient Research Record™ (OQR2™) when the triathlon participant plans meet all legal requirements for administering health, triathlon and nutrition research.

Along with an adult or adolescent entry qualification evaluations and parental authorization, a fitness research record may be generated with any sports medicine and exercise science entry within the World Fitness Maintenance Network of the Research institution.

An O'Baymetrics Quotient Research Record™ (OQR2™) must meet any legal requirements of a local, state or Federal Department of Justice, Health and Human Services standards of a given country when necessary for the completion and submission of a triathlon participant's training form.

Records may not be added to the personal or professional records of a triathlon participant without the administrative authorization from either the medical or exercise physiologist and dietician/nutritionist director.

In some embodiments, the O'Bay System may include the onsite and mobile cycling fitness maintenance periodization program. The cycling fitness maintenance program provides onsite and mobile cycling fitness and/or nutrition periodization testing, analysis and training and education, counseling, one-on-one, group, computer-based or video instruction means to the cycling fitness and/or nutrition periodization participants within our fitness maintenance facility.

The cycling fitness maintenance program may generate Individualized Cycling Fitness Maintenance Plan (ICP™) through the onsite and mobile and cycling fitness and/or nutrition periodization testing, analysis and training system services of the Research institution within our fitness facility.

The cycling fitness maintenance program may be administered throughout all stages of the Individualized Cycling Fitness Maintenance Plan™ (ICP™) evaluation, education and training stages of the Research institution within our fitness facility.

By creating and administering Individualized Cycling Fitness Maintenance Plan (ICP™), the cycling fitness maintenance program may provide the cycling fitness and/or nutrition periodization testing, analysis and training means to the cycling fitness maintenance participants interested in their meaningful competitive cycling fitness maintenance endeavor.

Embodied in the computer software program of the World Fitness Maintenance Network, the TTIP program enables research institution employees and supervisors to track systemic entries. The entry on the World Fitness Maintenance Network may create an O'Baymetrics Quotient Record™ (OQR™) based on the participant's profile, tracking, over time and/or distance and/or calorie consumption and/or weight, the progress and perform during periodic evaluations and assessments.

Along with an adult or adolescent entry qualification evaluations and parental authorization, a fitness research record may be generated with any cycling fitness and/or nutrition periodization testing, analysis and training entry form within the World Fitness Maintenance Network of the Research institution.

An adolescent or adult Individualized Cycling Fitness Maintenance Plan (ICP™) and an O'Baymetrics Quotient Record™ (OQR™) may be automated and integrated into the World Fitness Maintenance Network of the Research institution as an O'Baymetrics Quotient Research Record™ (OQR2™) when the cycling fitness and/or nutrition periodization participant plans meet all legal requirements for administering health, fitness maintenance research.

An O'Baymetrics Quotient Research Record™ (OQR2™) must meet any legal requirements of a local, state or Federal Department of Justice, Health and Human Services standards of a given country when necessary for the completion and submission of a cycling fitness and/or nutrition periodization participant's training form.

Records may not be added to the personal or professional records of a cycling fitness and/or nutrition periodization participant without the administrative authorization from either the medical or exercise physiologist and dietician/nutritionist director.

In some embodiments, the O'Bay System may include the World Fitness Maintenance Network™. The World Fitness Maintenance Network may be an online, fitness and/or nutrition record and search engine system, server and method receives input from the exercise physiologist and dietician/nutritionist or certified personal trainers' terminal regarding fitness and/or nutrition information with a description of associated fitness maintenance products or services.

In some embodiments, the O'Bay System may include the Triathlon and Nutrition Periodization Training Program. The Triathlon and Nutrition Periodization Training Program provides an individualized, onsite and mobile triathlon and nutrition training, education, counseling, one-on-one, group, computer-based or video instruction means to local overweight and obese adolescent and adult fitness participants in our Oregon Bay Fitness facility.

The Triathlon and Nutrition Periodization Training Program may generate an Individualized Triathlon, Fitness Maintenance Plan, (ITP™) (IFP™) (INP™), respectively, through the onsite and mobile sports medicine and exercise science services of the Research institution within our fitness maintenance facility.

The Triathlon and Nutrition Periodization Training Program may be administered throughout all stages of an Individualized Triathlon, Fitness Maintenance Plan, (ITP™) (IFP™) (INP™) evaluation, education and training stages by the Research institution.

By creating and administering an individualized Nutrition Plan (ITP™), the AMNS may provide the nutrition periodization means to the overweight and obese fitness participants interested in their meaningful triathlon and nutrition endeavor.

Embodied in the computer software program of the World Fitness Maintenance Network, the Triathlon and Nutrition Periodization Training Program enables Research institution employees and supervisors to track systemic entries. The entry on the World Fitness Maintenance Network may create an O'Baymetrics Quotient Record™ (OQR™) based on the fitness participant's profile, tracking, over time and/or distance and/or calorie consumption and/or weight, the progress and performs during periodic evaluations and assessments.

Along with an overweight and obese adult or adolescent entry qualification evaluations and parental authorization, an O'Baymetrics Quotient Research Record may be generated with any sports medicine and exercise science entry within the World Fitness Maintenance Network of the research institution.

Any individualized Nutrition Plan (INP™) and O'Baymetrics Quotient Record™ (OQR™) may be automated and integrated into the World Fitness Maintenance Network of the Research institution as an O'Baymetrics Quotient Research Record™ (OQR2™) when the fitness participant plans meet all legal requirements for administering health, fitness maintenance research.

An O'Baymetrics Quotient Research Record™ (OQR2™) must meet any legal requirements of a local, state or Federal Department of Justice, Health and Human Services standards of a given country when necessary for the completion and submission of triathlon and nutrition participant's training form.

All records may not be added to the personal or professional records of a triathlon fitness and/or nutrition periodization participant without the administrative authorization from either the medical or exercise physiologist and dietician/nutritionist director.

In some embodiments, the O'Bay System may include an On-demand Fitness Maintenance Distribution system comprises a secure server hosting an on-demand fitness and/or nutrition database of sports medicine physician, exercise science physiologist, dietician, nutritionist, certified personal trainer, cycling wind tunnel experts and other fitness and/or nutrition service providers and a customer's electronic data processing device on which an on-demand fitness and/or nutrition application may be loaded. the customer's electronic data processing device may be configured to execute the on-demand fitness and/or nutrition application to perform operations including: storing a local on-demand fitness and/or nutrition database containing a portion or all of the on-demand fitness and/or nutrition items stored in the on-demand fitness and/or nutrition database at the secure server; selecting an on-demand fitness and/or nutrition item stored in the local on-demand fitness and/or nutrition database; displaying the selected on-demand fitness and/or nutrition item or causing the selected on-demand fitness and/or nutrition item to be displayed by a viewer application executing on the electronic data processing device; constructing an email including a hyperlink for downloading or streaming the selected on-demand fitness and/or nutrition item from the on-demand fitness and/or nutrition database of the secure server; and sending the constructed email to a recipient or causing the constructed email to be sent to the recipient by an email application executing on the electronic data processing device.

II. Platform Configuration

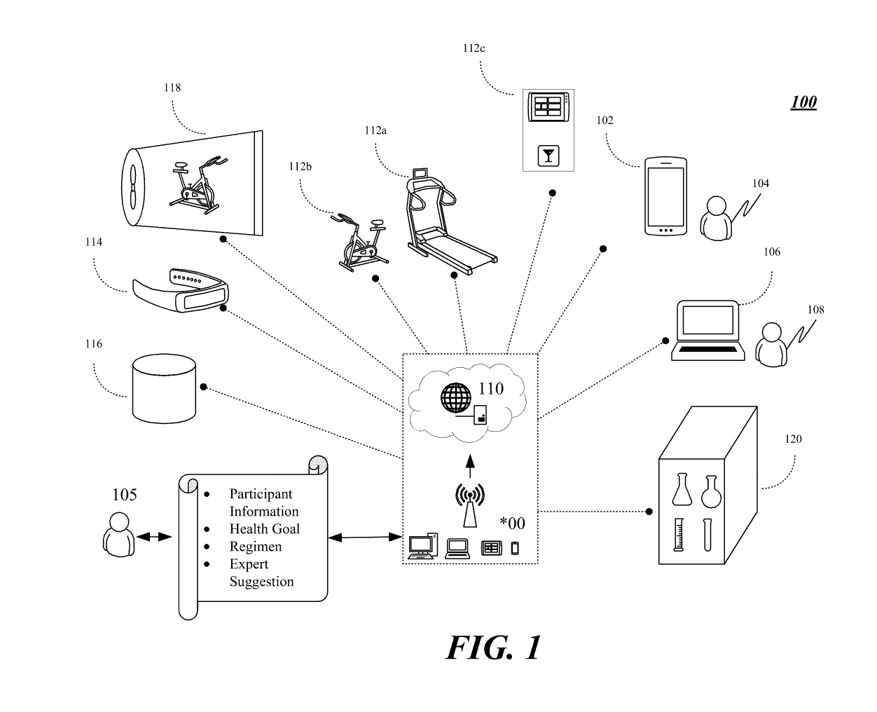

FIG. 1 is an illustration of a platform consistent with various embodiments of the present disclosure. By way of non-limiting example, the online platform 700 for facilitating maintenance of health may be hosted on a centralized server 110, such as, for example, a cloud computing service. The centralized server may communicate with other network entities, such as, for example, a mobile device (such as a smartphone, a laptop, a tablet computer etc.) and other electronic devices (such as desktop computers, etc.) over a communication network 112, such as, but not limited to, the Internet. Further, users of the platform may include health maintenance user 104 (such as healthy individuals, individuals with low/high Body Mass Index (BMI), individuals undergoing rehabilitation, athletes etc.), a health maintenance expert 108 (such as exercise physiologist, dietician, nutritionist director, clinician, physiotherapist, sports medicine consultant, trainer, coach, psychologist, psychiatrist etc.). Accordingly, electronic devices operated by the one or more individuals may be in communication with the platform. For example, the platform may be in communication with an electronic device 102 (e.g. smartphone, tablet computer, laptop computer, desktop computer, wearable computer etc.) operated by the health maintenance user 104 and an electronic device 106 (e.g. smartphone, tablet computer, laptop computer, desktop computer, wearable computer etc.) operated by the health maintenance expert 108.

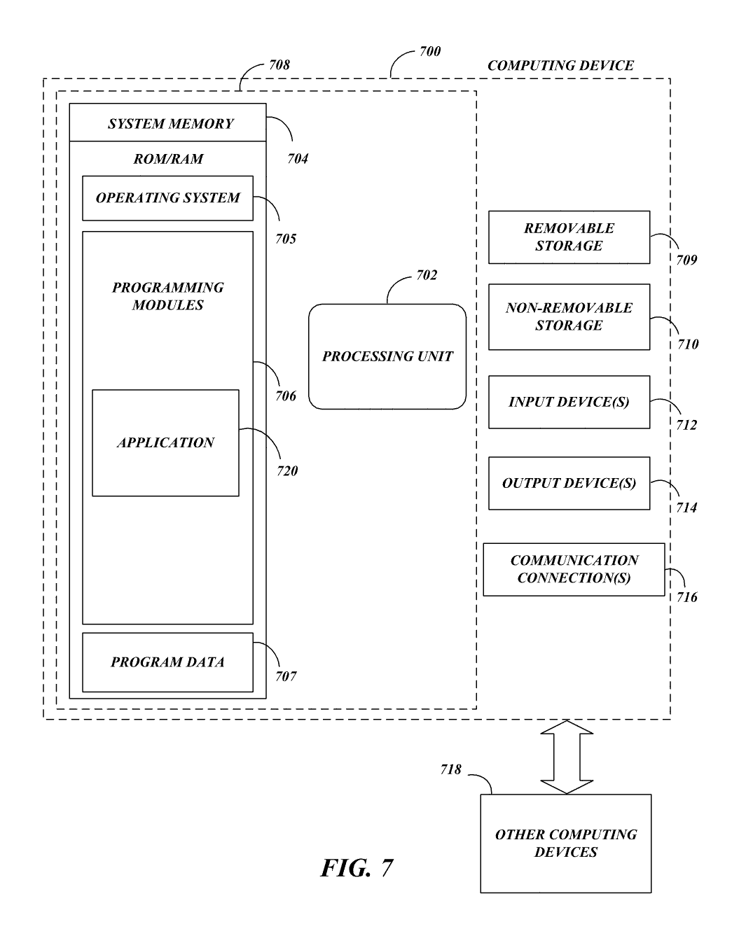

A user 105, such as the health maintenance user and/or the health maintenance expert, may access platform 100 through a software application. The software application may be embodied as, for example, but not be limited to, a website, a web application, a desktop application, and a mobile application compatible with a computing device 700, illustrated in FIG. 7. One possible embodiment of the software application may be provided by O'BAY™ products and services. Accordingly, in an instance, the user 105 may be enabled to provide participant information that may include, but is not limited to, any information that may facilitate management of a health of the health maintenance user. The health may correspond to one or more of a physical health and a mental/psychological health. For example, the health maintenance user's name, age, gender, height, weight, health condition (e.g. acute/chronic medical condition), health maintenance goals, health maintenance schedules, nutritional preferences (e.g. vegetarian, vegan etc.) and so on. Further, in another instance, the user 105 may be the health maintenance expert 108. Accordingly, the user 105 may be enabled to provide any information that facilitate management of the health maintenance user, such as for example, but not limited to, regimen and expert suggestions. Further, the user 105 may be presented with information generated by the online platform 100, such as, but not limited to, suggested health maintenance goals, suggested health maintenance schedules, regimen, nutrition periodization plans, performance assessments, and so on.

Further, in some embodiments, the online platform 100 may be configured to communicate with one or more health maintenance devices 112 such as, but not limited to, fitness maintenance devices (e.g. treadmill 112a, exercise bike 112b) and nutrition maintenance devices (e.g. nutrition dispenser 112c). Accordingly, the one or more health maintenance devices 112 may be configured to transmit operational data corresponding to a corresponding functioning. For example, the online platform 100 may be configured to receive operational data such as speed, elevation, time period of use, etc. from the treadmill 112a, distance travelled, speed, acceleration etc. from exercise bike 112b and type/quantity of food dispensed, amount of calories, type/quantity of nutrients (minerals, vitamins, performance enhancers etc.) from the nutrition dispenser 112c. In some embodiments, the one or more health maintenance devices 112 may be configured to maintain and/or enhance a mental/psychological health of the health maintenance user 104. For example, the health maintenance devices 112 may be configured to enhance concentration skills, stress management, cognitive abilities and so on.

In addition, in some embodiments, the online platform 100 may also receive user data corresponding to the health maintenance user 104 from the one or more health maintenance devices 112. For example, the treadmill 112a may include sensors configured to capture a state (e.g. physical state, physiological state and/or psychological state) of the health maintenance user 104. Further, the online platform 100 may be configured to receive information entered by the health maintenance user 104 into the one or more health maintenance devices 112 such as, but not limited to, health maintenance goals, health maintenance schedules etc. Additionally, in some embodiments, the online platform 100 may be configured to obtain data about the state of the health maintenance user 104 from one or more wearable devices 114 either directly and/or mediated through the one or more health maintenance devices 112.

Further, the online platform 100 may be configured to transmit control codes to the one or more health maintenance devices 112 in order to remotely control an operation of the one or more health maintenance devices 112. For example, the control codes may indicate a difficulty level (speed, elevation, time-period etc.) and/or a training weight associated with the one or more health maintenance devices 112. Similarly, the control codes may also control dispensing of type/quantity of food, amount of calories, type/quantity of nutrients (minerals, vitamins, performance enhancers etc.) from the nutrition dispenser 112c. Accordingly, the one or more health maintenance devices 112 may be configured to receive the control codes and effect a corresponding control action (e.g. increase/decrease an operational parameter such as speed, elevation, time-period, quantity of nutrient etc.).

In addition, the online platform 100 may also be configured to transmit information (such as, but not limited to, suggested health maintenance goals, regimen, expert suggestions etc.) presentable to the health maintenance user 104 through the one or more health maintenance devices 112. Further, in some embodiments, the online platform 100 may be configured to transmit virtual simulation data to the health maintenance user 104, presentable through one or more of the one or more health maintenance devices 112 and the electronic device 102. Such virtual simulation data may be generated by the online platform 100 based on one or more of the user data (e.g. the state of the health maintenance user), the operational data of the one or more health maintenance devices 112 and information received from the health maintenance expert 108. Similarly, virtual simulation data may be generated by the online platform 100 for presenting to the health maintenance expert 108 (such as, for example, a trainee) in order to train the health maintenance expert 108.

Further, the online platform 100 may be configured to communicate with a health maintenance database 116 configured to store any kind of information capable of facilitating management of the one or more health maintenance devices 112, the health maintenance user 102 and/or the health maintenance expert 108. For example, the health maintenance database 116 may be configured to store performance assessments of a plurality of health maintenance users, such as health maintenance user 104. Further, the health maintenance database 116 may also be configured to receive and store user data generated based on monitoring at least one of the one or more health maintenance devices 112 and the health maintenance user 104. In addition, the health maintenance database 116 may be configured to store demographical information, behavioral information, medical information, and so on associated with the plurality of health maintenance users. Further, the health maintenance database 116 may be configured to store information regarding a plurality of health maintenance experts, such as health maintenance expert 108 and a plurality of health maintenances devices, such as the one or more health maintenance devices 112. For example, the health maintenance database 116 may include demographical information, certifications, professional experience, awards/recognitions associated with the plurality of health maintenance experts. Further, the health maintenance database 116 may include technical information relating to the plurality of health maintenance devices, such as operational data, control codes, product features, etc. Additionally, the health maintenance database 116 may include results of analysis (e.g. statistical trends, best practices etc.) performed on one or more information stored on the health maintenance database 116. Further, the health maintenance database 116 may include the virtual simulation data for enhancing experience, knowledge and/or skills of one or more of the plurality of health maintenance users and the plurality of health maintenance experts.

In addition, in some embodiments, the online platform 100 may be configured to communicate with research equipment, such as but not limited to, cycling wind tunnel 118 and clinical/dietary laboratory equipment 120. As a result, the online platform 100 may be able to use the information received from the research equipment in order to provide health maintenance suggestions to one or more of the health maintenance user 104 and the health maintenance expert 108. Further, in some embodiments, the online platform 100 may also be able to control operation of the one or more health maintenance devices 112 based on information received from the research equipment. For instance, the clinical laboratory equipment 120 may transmit information to the online platform 100 indicating, for example, a blood constituent value of the health maintenance user 104. Accordingly, the online platform 100 may be configured to control operation of the one or more health maintenance devices 112 (such as, for example, nutrition dispenser 112c) in order to improve and/or maintain a health of the health maintenance user 104. Likewise, the online platform 100 may also be configured to receive research data from the research equipment such as the cycling wind tunnel and accordingly provide suggestions to the health maintenance participant such as, for example, a cyclist in order to improve and/or maintain cycling performance.

Accordingly, the online platform 100 may be configured to facilitate provisioning of a comprehensive management of health of the plurality of health maintenance users. For example, in some embodiments, as a result of the integration of the online platform 100 with the health maintenance expert 108, the one or more health maintenance devices 112, the one or more wearable devices 114, the health maintenance database 116 and the research equipment (e.g. cycling wind tunnel 118 and laboratory equipment 120), the health maintenance user 104 may be provided with enhanced health maintenance.

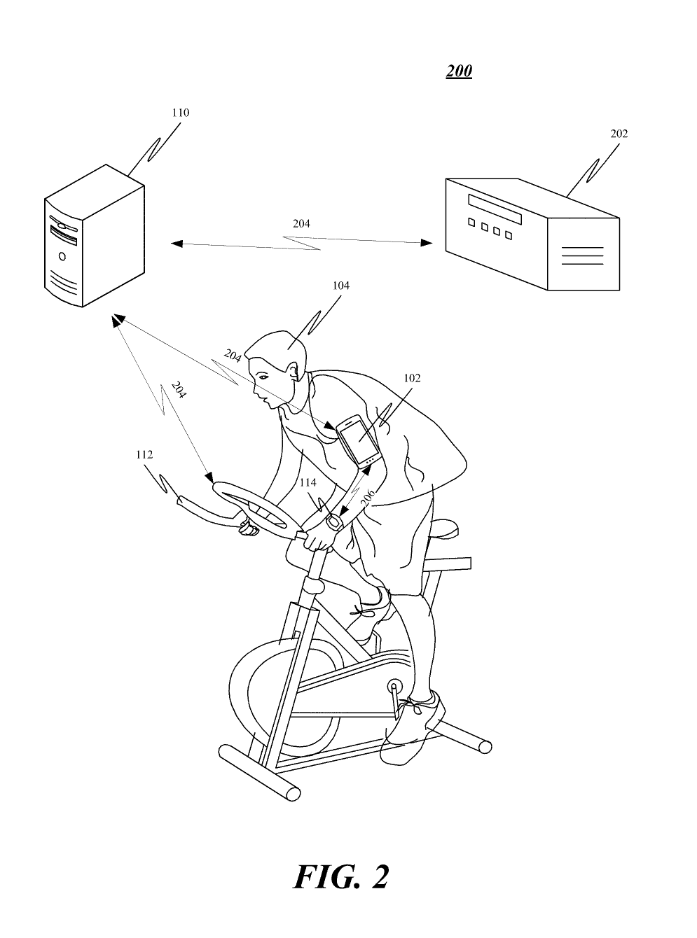

FIG. 2 illustrates a system 200 for facilitating operation of a health maintenance device 112, in accordance with some embodiments. The health maintenance device 112 may be configured to provide a physical activity performable by the health maintenance user 104 in order to enhance health including, but not limited to, one or more of a preferable physiological condition and physical performance level. For instance, as illustrated, the health maintenance 112 may be an exercise bike. Further, the system 200 may include a room automation unit 202 configured to electrically control an operational state of one or more electrical appliances associated with the exercise bike. For example, the room automation unit 202 may be configured to control a temperature, humidity level, pressure, air flow etc. of a room in which the exercise bike is situated. Further, the room automation unit 202 may be in communication with a server 110 of the online platform 100 through a communication network 204, such as, but not limited to, the Internet. Likewise, each of the exercise bike 112 and the electronic device 102 may also be in communication with the server 110 through the communication network 204. Further, a wearable device 114 may in some embodiments, be in a near-field communication 206 with the electronic device 102. For example, the wearable device 114 may be paired with the electronic device 102 (such as a smartphone) through Bluetooth communication channel. Accordingly, the server 110 may be able to receive sensor data (e.g. heart rate, blood pressure, acceleration data etc.) from the wearable device 114 through the electronic device 102. Accordingly, in some embodiments, the server 110 of the online platform may receive an input such as one or more of sensor data from the wearable device 114 representing a physical, physiological and/or mental state of the health maintenance user 104, environmental data from the room automation unit 202 and operational data (e.g. speed, elevation, time-period of use etc.) from the exercise bike 112. Further, the server 110 may be configured to perform an analysis of the input and generate an output including one or more of, health maintenance suggestions presentable to the health maintenance user on one or more of the exercise bike 112, the electronic device 102 and the wearable device 114, control codes for controlling an operational state of the exercise bike 112 (e.g. speed limit, elevation limit, time-period of use limit etc.) and control codes for controlling the room automation unit 202 in order to effect a change in an environmental factor associated with the exercise bike 112.

As an exemplary application of the system 200, the server 110 may receive user data, such as a medical condition of the health maintenance user 104. Accordingly, based on the medical condition and/or suggestions received from the health maintenance expert 108, the server 110 may be configured to generate one or more boundaries corresponding to one or more operational parameters of the exercise bike 112. Further, by receiving operational data from the exercise bike 112 in real-time, the server 110 may be able to detect a deviation of an operational state of the exercise bike 112 from the one or more boundaries. Accordingly, based on the deviation detected, the server 110 may be able to transmit an alert to the health maintenance user 102 and/or the health maintenance expert 108. Additionally, the server 110 may also be configured to transmit control codes to one or more of the exercise bike 112 and the room automation unit 202 for effecting a safety action (e.g. reducing a difficulty level by decreasing speed/resistance, deactivating one or more functionalities of the exercise bike 112 etc.).

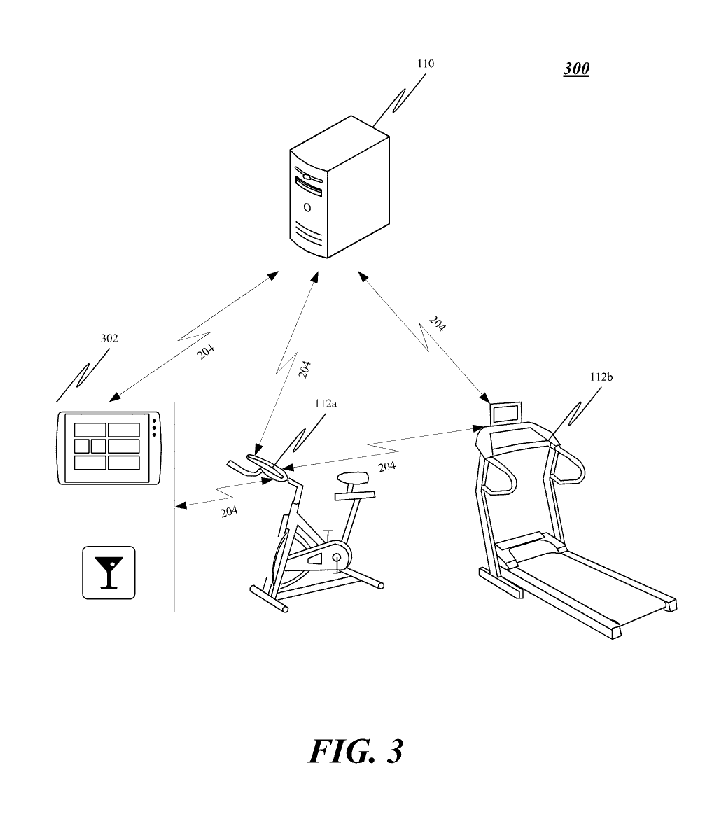

FIG. 3 illustrates a system 300 for facilitating operation of a plurality of health maintenance devices, in accordance with some embodiments. The plurality of health maintenance devices may include, for example, but not limited to, an exercise bike 112a, a treadmill 112b and a nutrition dispenser 302, each of which may be in communication with the server 110 of the online platform 100 through the communication network 204. In addition, in some embodiments, the plurality of health maintenance devices may be in communication amongst each other through, for example, the communication network 204. Accordingly, for instance, the treadmill 112b and the exercise bike 112a may transmit and/or receive various health maintenance related data such as, but not limited to, a quantity of calories burnt, amount of energy expended, power produced, a time period of usage, a type of exercise activity performed, a time and place where the exercise activity was performed, physiological condition of the health maintenance user 104 before, during and after performing the exercise activity and so on. As a result, a health maintenance user 104 may be able to use the plurality of health maintenance devices seamlessly while pursuing a health maintenance goal. In other words, as a result of exchange of health maintenance related data, each of the plurality of health maintenance devices may automatically adapt its operational state in accordance with health maintenance activity (such as exercise) performed by the health maintenance user 104 on other health maintenance devices. Consequently, for example, a first health maintenance device may be able to track a percentage of the health maintenance goal achieved during a first health maintenance session and transfer the percentage to a second health maintenance device. Further, the second health maintenance device may be configured to adjust one or more operational parameters based on the percentage of the health maintenance goal.

Similarly, in an instance, each of the exercise bike 112a and the treadmill 112b may transmit information regarding respective health maintenance sessions to the nutrition dispenser 112c. Accordingly, for example, the nutrition dispenser may be configured to dispense a type and quantity of nutrient based on analysis of the health maintenance sessions performed by the health maintenance user 104. For instance, over a period of time, such as a week, the health maintenance user 104 may be prescribed a specific nutrition intake as well as a specific training schedule including number of calories to be expended, an amount of weight to be gained or lost and so on. Accordingly, during the course of the week, as the health maintenance user 104 performs sessions over the plurality of health maintenance devices, information regarding the sessions may be transmitted to the nutrition dispenser 112c. Accordingly, the nutrition dispenser 112c may control a type and quantity of one or more nutrients dispensed to the health maintenance user 104 in order to meet the prescribed nutrition while considering the amount of calories/energy spent during the sessions. Further, in some embodiments, the nutrition dispenser 112c may receive a training schedule corresponding to the health maintenance user 104. Accordingly, the nutrition dispenser 112c may be able to automatically perform nutrition periodization to ensure that the health maintenance user 104 is provided nutrition in such a way that a weight of the user is suitable for undergoing the training before and during the training period.

As will be detailed with reference to FIG. 7 below, the computing device through which the online platform may be accessed may comprise, but not be limited to, for example, a desktop computer, laptop, a tablet, or mobile telecommunications device. As will be detailed with reference to FIG. 7 below, the computing device through which the platform may be accessed may comprise, but not be limited to, for example, a desktop computer, laptop, a tablet, or mobile telecommunications device. Though the present disclosure is written with reference to a mobile telecommunications device, it should be understood that any computing device may be employed to provide the various embodiments disclosed herein.

III. Platform Operation

Although methods 400, 500 and 600 have been described to be performed by platform 100, it should be understood that computing device 700 may be used to perform the various stages of methods 400, 500 and 600. Furthermore, in some embodiments, different operations may be performed by different networked elements in operative communication with computing device 700. For example, server 110 may be employed in the performance of some or all of the stages in methods 400, 500 and 600. Moreover, server 110 may be configured much like computing device 700.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of methods 400, 500 and 600 will be described in greater detail below.

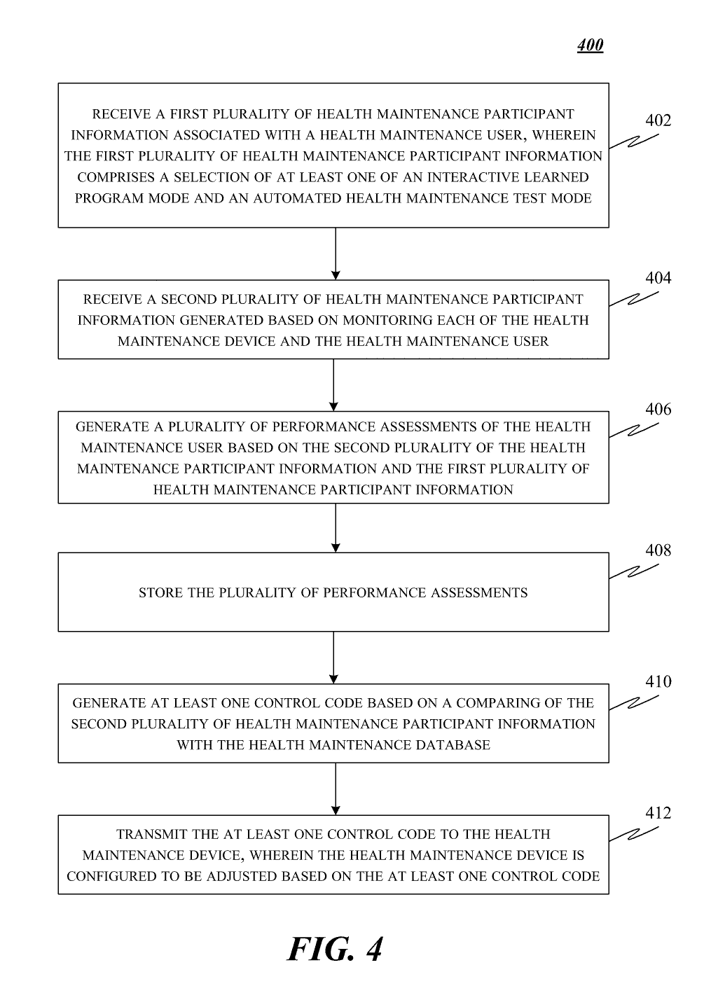

FIG. 4 illustrates a computer implemented method 400 of facilitating operation of a health maintenance device, in accordance with some embodiments. The computer implemented method 400 may include a stage 402 of receiving, using a communication device, a first plurality of health maintenance participant information associated with a health maintenance user. Further, the first plurality of health maintenance participant information may include a selection of one or more of an interactive learned program mode and an automated health maintenance test mode. In some embodiments, the first plurality of health maintenance information may include one or more of user data, at least one goal and a selection of an operation mode of the health maintenance device.

Furthermore, the computer implemented method 400 may include a stage 404 of receiving, using the communication device, a second plurality of health maintenance participant information generated based on monitoring each of the health maintenance device and the health maintenance user. In some embodiments, the second plurality of health maintenance participant information may include one or more of a weight, a speed and a distance traveled associated with the health maintenance device. Further, the health maintenance device may include at least one sensor configured to detect one or more of the weight, the speed and the distance travelled.

Additionally, the computer implemented method 400 may include a stage 406 of generating, using a processing device, a plurality of performance assessments of the health maintenance user corresponding to a plurality of health maintenance sessions of the health maintenance device. Further, each performance assessment may be generated based on the second plurality of the health maintenance participant information and the first plurality of health maintenance participant information. In some embodiments, a performance assessment of the plurality of performance assessments may include an energy expenditure associated with the at least one goal. In some embodiments, the performance assessment further may include a percentage of reaching the at least one goal. Further, the percentage may be determined based on dividing the energy expenditure by one or more of a weight and a time length of use of the health maintenance device.

In some embodiments, the computer implemented method may further include: generating, using the processing device, a suggested goal based on a comparison of the percentage with a predetermined low; and transmitting, using the communication device, the suggested goal to the health maintenance device.

Furthermore, the computer implemented method 400 may include a stage 408 of storing, using a storage device, the plurality of performance assessments. Additionally, the computer implemented method 400 may include a stage 410 of generating, using the processing device, at least one control code based on a comparing of the second plurality of health maintenance participant information with the health maintenance database.

Further, the computer implemented method 400 may include a stage 412 of transmitting, using the communication device, the at least one control code to the health maintenance device. Furthermore, the health maintenance device may be configured to be adjusted based on the at least one control code. In some embodiments, the at least one control code may be configured to adjust one or more of a difficulty level and an environmental factor associated with the health maintenance device. In some embodiments, generating the at least one control code may be further based on the first plurality of health maintenance participant information.

In some embodiments, the computer implemented method may further include: generating, using the processing device, at least one predetermined health maintenance boundary associated with the health maintenance device. Further, the at least one predetermined health maintenance boundary may be generated based on the user data; and transmitting, using the communication device, the at least one predetermined health maintenance boundary to the health maintenance device. Further, the health maintenance device may be configured to generate an alert based on an operational value of the health maintenance device exceeding the at least one predetermined health maintenance boundary.

In some embodiments, the user data may include a physical condition. Further, the predetermined health maintenance boundary may be further associated with physiological data of the health maintenance user. Further, the health maintenance device may be further configured to detect physiological data of the health maintenance user.

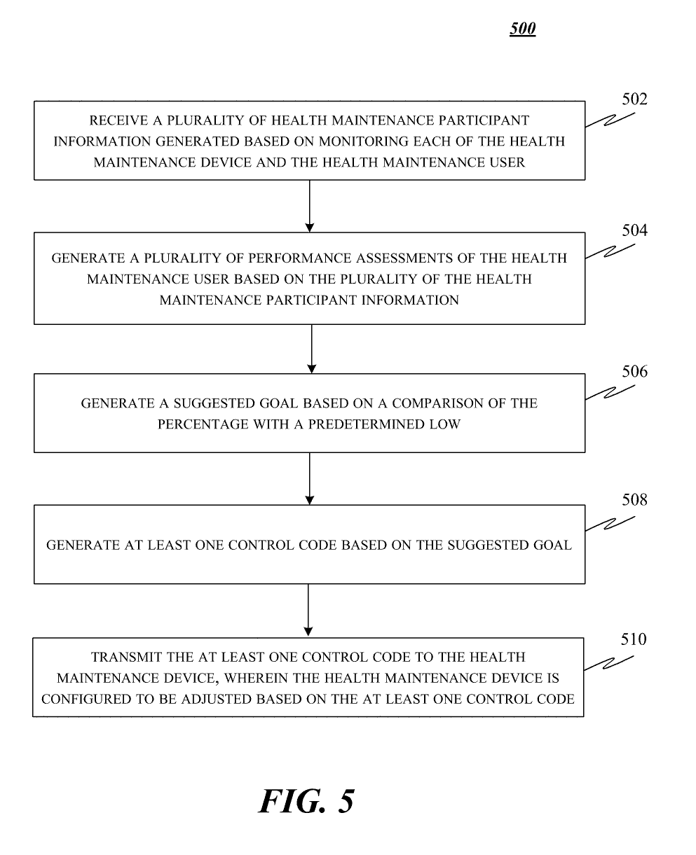

FIG. 5 illustrates a method 500 of facilitating operation of a health maintenance device by suggesting a health maintenance goal, in accordance with some embodiments. The method 500 may be implemented using a computer. The method 500 may include a stage of receiving a plurality of health maintenance participant information generated based on monitoring each of the health maintenance device 112 and the health maintenance user 104. Further, the method 500 may include a stage 504 of generating a plurality of performance assessments of the health maintenance user 104 based on the plurality of the health maintenance participant information. Furthermore, the method 500 may include a stage 506 of generating a suggested goal based on a comparison of the percentage with a predetermined low. Additionally, the method 500 may include a stage 508 of generating at least one control code based on the suggested goal. Further, the method 500 may include a stage 510 of transmitting the at least one control code to the health maintenance device 112. Furthermore, the health maintenance device 112 may be configured to be adjusted based on the at least one control code. Accordingly, a suggested goal may be set on the health maintenance device 112 based on health maintenance participant information such as age, gender, medical condition, etc. and also on performance of the health maintenance user 104.

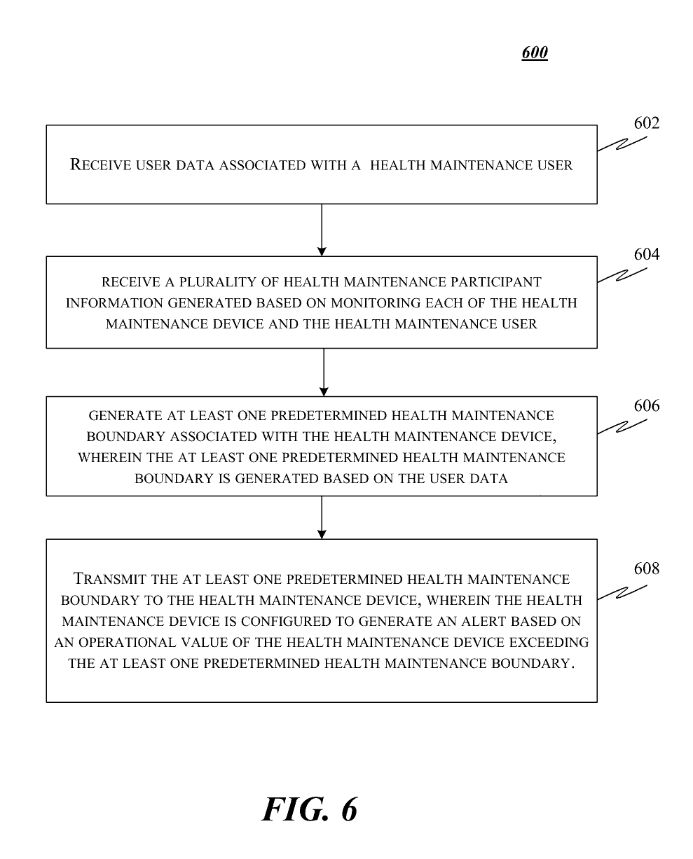

FIG. 6 illustrates a method 600 of facilitating operation of a health maintenance device based on user data (such as, but not limited to, clinical considerations) associated with a user, in accordance with some embodiments. The method 600 may be implemented using a computer. The method 600 may include a stage 602 of receiving user data associated with the health maintenance user 104. Further, the method 600 may include a stage 604 of receiving a plurality of health maintenance participant information generated based on monitoring each of the health maintenance device 112 and the health maintenance user 104. Furthermore, the method 600 may include a stage 606 of generating at least one predetermined health maintenance boundary associated with the health maintenance device. Further, the at least one predetermined health maintenance boundary may be generated based on the user data. Additionally, the method 600 may include a step 608 of transmitting the at least one predetermined health maintenance boundary to the health maintenance device. The health maintenance device may be configured to generate an alert based on an operational value of the health maintenance device exceeding the at least one predetermined health maintenance boundary.

The disclosed methods, systems, devices and apparatus include at least the following aspects:

Aspect 1: The O'Bay System™ application is a secured, internal method of reviewing, analyzing, and prescribing a fitness and/or nutrition option with one or more fitness and/or nutrition options using a computer or artificial intelligence, and an exercise physiologist and dietician/nutritionist or certified personal trainer associated therewith, the exercise physiologist and dietician/nutritionist or certified personal trainer coordinating said interactive computer assisted method or artificial intelligence system with at least one medical physician, one exercise physiologist and at least one registered dietitian, prior to the prescribing the fitness and/or nutrition participant of the one or more fitness and/or nutrition regiments, said method comprising the steps of:

(a) Pre-selecting fitness and/or nutrition participants to obtain a preliminary set of fitness and/or nutrition participants' eligible for said interactive computer assisted method or artificial intelligence system responsive to first predetermined criteria;

(b) filtering the preliminary set of fitness and/or nutrition participants to identify and form a secondary set of fitness and/or nutrition participants from the preliminary set of fitness and/or nutrition participants having a greater likelihood of benefiting from said interactive computer assisted method or artificial intelligence system responsive to second predetermined criteria;

(c) Enrolling at least one fitness and/or nutrition participant from the secondary set of fitness and/or nutrition participants;

(d) Communicating, by the exercise physiologist and dietician/nutritionist or certified personal trainer, with at least one fitness participant to obtain information to assist the fitness user's exercise physiologist and dietician/nutritionist or certified personal trainer in determining whether at least one of fitness options and nutrition changes are appropriate;

(e) Preliminarily evaluating, by the exercise physiologist and dietician/nutritionist or certified personal trainer, whether the at least one of fitness options and nutrition changes are appropriate responsive to the information;

(f) Communicating, by the exercise physiologist and dietician/nutritionist or certified personal trainer, to a sports medicine or exercise physiologist M.D. or Ph.D., the at least one of fitness options and nutrition changes and the information;

(g) determining, by the exercise physiologist and dietician/nutritionist or certified personal trainer, whether the at least one of fitness options and nutrition changes are appropriate responsive to the information, and prescribing at least one of the at least one of fitness options and nutrition changes, at least one of other fitness options and nutrition changes, and no fitness options and nutrition changes for the at least one fitness participant;

(h) confirming, by the exercise physiologist and dietician/nutritionist or certified personal trainer, the prescribing by the sports medicine or exercise physiologist M.D. or Ph.D. with the registered dietitian for the at least one of the at least one of fitness options and nutrition changes, at least one of other fitness options and nutrition changes, and no fitness options and nutrition changes for at least one fitness participant, and receiving comments from the registered dietitian relating thereto; and

(i) Communicating, by the exercise physiologist and dietician/nutritionist or certified personal trainer, to the sports medicine or exercise physiologist M.D. or Ph.D., the comments received from the registered dietitian at least when the comments include a registered dietitian opinion with respect to the at least one of fitness options and nutrition changes and the information determined by the exercise physiologist and dietician/nutritionist or certified personal trainer.

Aspect 2: An interactive computer assisted method or artificial intelligence system per aspect 1, further comprising the step of confirming, by the exercise physiologist and dietician/nutritionist or certified personal trainer, the prescribing by the sports medicine or exercise physiologist M.D. or Ph.D. with the fitness and/or nutrition participant.

Aspect 3: An interactive computer assisted method or artificial intelligence system per aspect 1, further comprising the step of confirming, by exercise physiologist and dietician/nutritionist or certified personal trainer, the prescribing by the exercise physiologist with a registered dietitian for possible fitness and/or nutrition changes.

Aspect 4: An interactive computer assisted method or artificial intelligence system according to aspect 1, further comprising the step of communicating, by exercise physiologist and dietician/nutritionist or certified personal trainer, to a registered dietitian, to obtain additional information to further assist the fitness user's exercise physiologist and dietician/nutritionist or certified personal trainer in determining whether the at least one of fitness options and nutrition changes are appropriate.

Aspect 5: An interactive computer assisted method or artificial intelligence system according to aspect 1, wherein the information includes at least one of fitness demographics, the certified exercise physiologist and dietician/nutritionist or certified personal trainer(s), current fitness and nutrition regiments, fitness and nutrition usage pattern, over the counter fitness and nutrition use, fitness participant understanding of fitness goals, adverse effects, compliance history, medical history, history, pertinent laboratory work, fitness participant concerns and assessment, and fitness participant satisfaction assessment.

Aspect 6: An interactive computer assisted method or artificial intelligence system per aspect 1, further comprising the steps of confirming, by the exercise physiologist and dietician/nutritionist or certified personal trainer, the prescribing by the sports medicine or exercise physiologist M.D. or Ph.D. with a registered dietitian for possible fitness and/or nutrition changes to the fitness participant.

Aspect 7: An interactive computer assisted method or artificial intelligence system of reviewing, analyzing, and prescribing a fitness participant one or more fitness and/or nutrition options using a computer and an exercise physiologist and dietician/nutritionist or certified personal trainer associated therewith, exercise physiologist and dietician/nutritionist or certified personal trainer coordinating said interactive computer assisted method or artificial intelligence system with at least one medical physician, one sports medicine or exercise physiologist M.D. or Ph.D. and at least one registered dietitian, prior to the prescribing the fitness participant of the one or more fitness and/or nutrition regiments, said method comprising the steps of:

(a) Pre-selecting fitness participants to obtain a preliminary set of fitness participants' eligible for said interactive computer assisted method or artificial intelligence system responsive to first predetermined criteria;

(b) filtering the preliminary set of fitness participants to identify and form a secondary set of fitness participants from the preliminary set of fitness participants having a greater likelihood of benefiting from said interactive computer assisted method or artificial intelligence system responsive to second predetermined criteria;

(c) Enrolling, by at least one of the exercise physiologist and dietician/nutritionist or certified personal trainer using the computer and the computer, at least one fitness participant from the secondary set of fitness participants;

(d) communicating with the at least one fitness participant to obtain information to assist the fitness user's exercise physiologist and dietician/nutritionist or certified personal trainer in determining whether at least one of fitness options and nutrition changes are appropriate, the information including at least one of fitness participant demographics, fitness participants exercise physiologist and dietician/nutritionist or certified personal trainer(s), current fitness and nutrition regiments, fitness nutrition usage pattern, over the counter food and nutrition use, fitness participant understanding of fitness goals, adverse effects, compliance history, medical history, history, pertinent laboratory work, fitness participant concerns and assessment, and fitness participant satisfaction assessment;

(e) Communicating, by the exercise physiologist and dietician/nutritionist or certified personal trainer, to the registered dietitian, to obtain additional information to further assist the fitness user's exercise physiologist and dietician/nutritionist or certified personal trainer in determining whether the at least one of fitness options and nutrition changes are appropriate;

(f) Preliminarily evaluating, by the exercise physiologist and dietician/nutritionist or certified personal trainer, whether the at least one of fitness options and nutrition changes are appropriate responsive to the information and the additional information;

(g) Communicating, by the exercise physiologist and dietician/nutritionist or certified personal trainer, to a sports medicine or exercise physiologist M.D. or Ph.D., the at least one of fitness options and nutrition changes, the information, and the additional information;

(h) determining, by the exercise physiologist and dietician/nutritionist or certified personal trainer, whether the at least one of fitness options and nutrition changes are appropriate responsive to the information, and prescribing at least one of the at least one of fitness options and nutrition changes, at least one of other fitness options and nutrition changes, and no fitness options and nutrition changes for the at least one fitness participant;

(i) Confirming, by the exercise physiologist and dietician/nutritionist or certified personal trainer, the prescribing by the sports medicine or exercise physiologist M.D. or Ph.D. with the fitness participant;

(j) Confirming, by the exercise physiologist and dietician/nutritionist or certified personal trainer, the prescribing by the sports medicine or exercise physiologist M.D. or Ph.D. with the registered dietitian for the at least one of the at least one of fitness options and nutrition changes, at least one of other fitness options and nutrition changes, and no fitness options and nutrition changes for the at least one fitness participant, and receiving comments from the registered dietitian relating thereto; and

(k) Communicating, by the exercise physiologist and dietician/nutritionist or certified personal trainer, to the sports medicine or exercise physiologist M.D. or Ph.D., the comments received from the registered dietitian at least when the comments include a registered dietitian opinion with respect to the at least one of fitness options and nutrition changes and the information determined by the exercise physiologist and dietician/nutritionist or certified personal trainer; and

(l) Fitness and/or nutrition to the fitness participant responsive to at least one of the fitness and/or nutrition changes and the other fitness and/or nutrition changes.

Aspect 8: An interactive computer assisted method or artificial intelligence system of reviewing, analyzing, and prescribing a fitness participant one or more fitness and/or nutrition options using a computer or artificial intelligence, and an exercise physiologist and dietician/nutritionist or certified personal trainer associated therewith, exercise physiologist and dietician/nutritionist or certified personal trainer coordinating said interactive computer assisted method or artificial intelligence system with at least one Medical physician, one sports medicine or exercise physiologist M.D. or Ph.D. and at least one registered dietitian, prior to the prescribing the fitness participant of the one or more fitness and/or nutrition regiments, said method comprising the steps of:

(a) Pre-selecting fitness participants to obtain a preliminary set of fitness participants' eligible for said interactive computer assisted method or artificial intelligence system responsive to first predetermined criteria;

(b) filtering the preliminary set of fitness participants to identify and form a secondary set of fitness participants from the preliminary set of fitness participants having a greater likelihood of benefiting from said interactive computer assisted method or artificial intelligence system responsive to second predetermined criteria;

(c) Enrolling, by the computer, at least one fitness participant from the secondary set of fitness participants;

(d) Communicating with the at least one fitness participant to obtain information to assist the fitness user's exercise physiologist and dietician/nutritionist or certified personal trainer in determining whether at least one of fitness options and nutrition changes are appropriate;

(e) Preliminarily evaluating whether the at least one of fitness options and nutrition changes are appropriate responsive to the information;

(f) Communicating by the computer, to an exercise physiologist and dietician/nutritionist or certified personal trainer, the at least one of fitness options and nutrition changes and the information; and

(g) determining, by the exercise physiologist and dietician/nutritionist or certified personal trainer, whether the at least one of fitness options and nutrition changes are appropriate responsive to the information, and prescribing at least one of the at least one of fitness options and nutrition changes, at least one of other fitness options and nutrition changes, and no fitness options and nutrition changes for the at least one fitness participant; and

(h) confirming, by the exercise physiologist and dietician/nutritionist or certified personal trainer, the prescribing by the sports medicine or exercise physiologist M.D. or Ph.D. with the registered dietitian for the at least one of the at least one of fitness options and nutrition changes, at least one of other fitness options and nutrition changes, and no fitness options and nutrition changes for the at least one fitness participant, and receiving comments from the registered dietitian relating thereto; and

(i) Communicating, by the exercise physiologist and dietician/nutritionist or certified personal trainer, to the sports medicine or exercise physiologist M.D. or Ph.D., the comments received from the registered dietitian at least when the comments include a registered dietitian opinion with respect to the at least one of fitness options and nutrition changes and the information determined by the exercise physiologist and dietician/nutritionist or certified personal trainer.

Aspect 9: An interactive computer assisted method or artificial intelligence system of reviewing, analyzing, and prescribing a fitness participant one or more fitness and/or nutrition options using a computer or artificial intelligence, and an exercise physiologist and dietician/nutritionist or certified personal trainer associated therewith, exercise physiologist and dietician/nutritionist or certified personal trainer coordinating said interactive computer assisted method or artificial intelligence system with at least one Medical physician, one sports medicine or exercise physiologist M.D. or Ph.D. and at least one registered dietitian, prior to the prescribing the fitness participant of the one or more fitness and/or nutrition regiments, said method comprising the steps of:

(a) Pre-selecting fitness participants to obtain a preliminary set of fitness participants' eligible for said interactive computer assisted method or artificial intelligence system responsive to first predetermined criteria;

(b) filtering the preliminary set of fitness participants to identify and form a secondary set of fitness participants from the preliminary set of fitness participants having a greater likelihood of benefiting from said interactive computer assisted method or artificial intelligence system responsive to second predetermined criteria;

(c) Enrolling, by the at least one of the exercise physiologist and dietician/nutritionist or certified personal trainer and the computer, at least one fitness participant from the secondary set of fitness participants;

(d) Communicating, by the at least one of the exercise physiologist and dietician/nutritionist or certified personal trainer and the computer, with the at least one fitness participant to obtain information to assist the fitness user's exercise physiologist and dietician/nutritionist or certified personal trainer in determining whether at least one of fitness options and nutrition changes are appropriate;

(e) Preliminarily evaluating, by the at least one of the exercise physiologist and dietician/nutritionist or certified personal trainer and the computer, whether the at least one of fitness options and nutrition changes are appropriate responsive to the information;

(f) Communicating, by the at least one of the exercise physiologist and dietician/nutritionist or certified personal trainer and the computer, to a sports medicine or exercise physiologist M.D. or Ph.D., the at least one of fitness options and nutrition changes and the information; and

(g) determining, by the exercise physiologist and dietician/nutritionist or certified personal trainer, whether the at least one of fitness options and nutrition changes are appropriate responsive to the information, and prescribing at least one of the at least one of fitness options and nutrition changes, at least one of other fitness options and nutrition changes, and no fitness options and nutrition changes for the at least one fitness participant;

(h) confirming, by the exercise physiologist and dietician/nutritionist or certified personal trainer, the prescribing by the sports medicine or exercise physiologist M.D. or Ph.D. with the registered dietitian for the at least one of the at least one of fitness options and nutrition changes, at least one of other fitness options and nutrition changes, and no fitness options and nutrition changes for at least one fitness participant, and receiving comments from the registered dietitian relating thereto; and

(i) Communicating, by the exercise physiologist and dietician/nutritionist or certified personal trainer, to the sports medicine or exercise physiologist M.D. or Ph.D., the comments received from the registered dietitian at least when the comments include a registered dietitian opinion with respect to the at least one of fitness options and nutrition changes and the information determined by the exercise physiologist and dietician/nutritionist or certified personal trainer.

Aspect 10: An interactive computer assisted method or artificial intelligence system per aspect 9, further comprising the step of confirming, by the exercise physiologist and dietician/nutritionist or certified personal trainer, the prescribing by the sports medicine or exercise physiologist M.D. or Ph.D. with the fitness participant.

Aspect 11: An interactive computer assisted method or artificial intelligence system per aspect 9, further comprising the step of confirming, by exercise physiologist and dietician/nutritionist or certified personal trainer, the prescribing by the sports medicine or exercise physiologist M.D. or Ph.D. with a registered dietitian for possible fitness and/or nutrition changes.

Aspect 12: An interactive computer assisted method or artificial intelligence system according to aspect 9, further comprising the step of communicating, by exercise physiologist and dietician/nutritionist or certified personal trainer, to a registered dietitian, to obtain additional information to further assist the fitness user's exercise physiologist and dietician/nutritionist or certified personal trainer in determining whether the at least one of fitness options and nutrition changes are appropriate.

Aspect 13: An interactive computer assisted method or artificial intelligence system according to aspect 9, wherein the information includes at least one of fitness participant demographics, the fitness participants' exercise physiologist and dietician/nutritionist or certified personal trainer(s), current fitness and nutrition regiments, fitness and nutrition usage pattern, over the counter fitness and nutrition usage, fitness participant understanding of fitness goals, adverse effects, compliance history, medical history, history, pertinent laboratory work, fitness participant concerns and assessment, and fitness participant satisfaction assessment.

Aspect 14: An interactive computer assisted method or artificial intelligence system per aspect 9, further comprising the steps of confirming, by the exercise physiologist and dietician/nutritionist or certified personal trainer, the prescribing by the sports medicine or exercise physiologist M.D. or Ph.D. with a registered dietitian of a mail order pharmacy for possible fitness and/or nutrition changes to the fitness participant.

Aspect 15: A computer program memory, storing computer instructions to implement an interactive computer assisted method or artificial intelligence system of reviewing, analyzing, and prescribing a fitness participant one or more fitness and/or nutrition options using a computer or artificial intelligence, and an exercise physiologist and dietician/nutritionist or certified personal trainer associated therewith, exercise physiologist and dietician/nutritionist or certified personal trainer coordinating said interactive computer assisted method or artificial intelligence system with at least one Medical physician, one sports medicine or exercise physiologist M.D. or Ph.D. and at least one registered dietitian, prior to the prescribing the fitness participant of the one or more fitness and/or nutrition regiments, the computer or artificial intelligence, instructions including:

(a) Pre-selecting fitness participants to obtain a preliminary set of fitness participants' eligible for said interactive computer assisted method or artificial intelligence system responsive to first predetermined criteria;

(b) filtering, by at least one of the exercise physiologist and dietician/nutritionist or certified personal trainer and the computer, the preliminary set of fitness participants to identify and form a secondary set of fitness participants from the preliminary set of fitness participants having a greater likelihood of benefiting from said interactive computer assisted method or artificial intelligence system responsive to second predetermined criteria;

(c) Enrolling, by the at least one of the exercise physiologist and dietician/nutritionist or certified personal trainer and the computer, at least one fitness participant from the secondary set of fitness participants;

(d) Communicating, by the at least one of the exercise physiologist and dietician/nutritionist or certified personal trainer and the computer, with the at least one fitness participant to obtain information to assist the fitness user's exercise physiologist and dietician/nutritionist or certified personal trainer in determining whether at least one of fitness options and nutrition changes are appropriate;

(e) Preliminarily evaluating, by the at least one of the exercise physiologist and dietician/nutritionist or certified personal trainer and the computer, whether the at least one of fitness options and nutrition changes are appropriate responsive to the information;

(f) Communicating, by the at least one of the exercise physiologist and dietician/nutritionist or certified personal trainer and the computer, to a sports medicine or exercise physiologist M.D. or Ph.D., the at least one of fitness options and nutrition changes and the information; and

(g) determining, by the exercise physiologist and dietician/nutritionist or certified personal trainer, whether the at least one of fitness options and nutrition changes are appropriate responsive to the information, and prescribing at least one of the at least one of fitness options and nutrition changes, at least one of other fitness options and nutrition changes, and no fitness options and nutrition changes for the at least one fitness participant;

(h) confirming, by the exercise physiologist and dietician/nutritionist or certified personal trainer, the prescribing by the sports medicine or exercise physiologist M.D. or Ph.D. with the registered dietitian for at least one of the at least one of fitness options and nutrition changes, at least one of other fitness options and nutrition changes, and no fitness options and nutrition changes for the at least one fitness participant, and receiving comments from the registered dietitian relating thereto; and

(i) Communicating, by the exercise physiologist and dietician/nutritionist or certified personal trainer, to the sports medicine or exercise physiologist M.D. or Ph.D., the comments received from the registered dietitian at least when the comments include a registered dietitian opinion with respect to at least one of fitness options and nutrition changes and the information determined by the exercise physiologist and dietician/nutritionist or certified personal trainer.

Aspect 16: A computer or artificial intelligence, program memory per aspect 15, further comprising the instruction of confirming, by the exercise physiologist and dietician/nutritionist or certified personal trainer, the prescribing by the sports medicine or exercise physiologist M.D. or Ph.D. with the fitness participant.

Aspect 17: A computer or artificial intelligence, program memory per aspect 15, further comprising the instruction of confirming, by exercise physiologist and dietician/nutritionist or certified personal trainer, the prescribing by the sports medicine or exercise physiologist M.D. or Ph.D. with a registered dietitian for possible fitness and/or nutrition changes.

Aspect 18: A computer or artificial intelligence, program memory per aspect 15, further comprising the instructions of communicating, by exercise physiologist and dietician/nutritionist or certified personal trainer, to a registered dietitian, to obtain additional information to further assist the fitness user's exercise physiologist and dietician/nutritionist or certified personal trainer in determining whether the at least one of fitness options and nutrition changes are appropriate.

Aspect 19: A computer or artificial intelligence, program memory according to aspect 15, wherein the information includes at least one of fitness participant demographics, fitness participants' exercise physiologist and dietician/nutritionist or certified personal trainer(s), current fitness and nutrition regiments, fitness and nutrition usage pattern, over the counter fitness and nutrition use, fitness participant understanding of fitness goals, adverse effects, compliance history, medical history, history, pertinent laboratory work, fitness participant concerns and assessment, and fitness participant satisfaction assessment.

Aspect 20: A computer or artificial intelligence, program memory per aspect 15, further comprising the instructions of confirming, by exercise physiologist and dietician/nutritionist or certified personal trainer, the prescribing by the sports medicine or exercise physiologist M.D. or Ph.D. with a registered dietitian for possible fitness and/or nutrition changes to the fitness participant.

Aspect 21: The O'Bay System is a surface monitoring method of fitness and/or nutrition comprising: entering by the fitness and/or nutrition participant with a fitness and/or nutrition device, prior to a use of the fitness and/or nutrition device by the fitness and/or nutrition participant, a first plurality of the fitness and/or nutrition participant information, which first plurality includes choosing a fitness and/or nutrition interactive learned program mode or an automated fitness and/or nutrition test mode; providing a fitness and/or nutrition control location that is remotely connected to the fitness and/or nutrition device through a communicative connection; receiving the first plurality of the fitness and/or nutrition participant information at the automated fitness and/or nutrition control location; monitoring a second plurality of the fitness and/or nutrition participant information during each use of the fitness and/or nutrition device by the of the fitness and/or nutrition participant; performing, at the automated fitness and/or nutrition control location, an assessment of a fitness and/or nutrition user during each use of a plurality of uses, based on a comparing of the second plurality of the fitness and/or nutrition participant information to the first plurality; incrementally generating, over the plurality of uses, fitness maintenance database including at least the plurality of fitness and/or nutrition performance assessments, wherein the fitness maintenance database is resident at the automated fitness and/or nutrition control location; and adjusting the use based on a comparing of the fitness maintenance database to the second plurality.

Aspect 22: The method of aspect 21, further comprising providing access by the fitness and/or nutrition participant to the fitness maintenance database during each use.

Aspect 23: The method of aspect 21, wherein said incrementally generating is over a fixed number of uses.

Aspect 24: The method of aspect 21, wherein said performing includes a calculating of energy expenditure necessary to reach at least one goal entered in the first plurality.

Aspect 25: The method of aspect 24, wherein said calculating further includes dividing the necessary energy expenditure by an expected weight or time length of the use, thereby generating a percentage of goals reached.

Aspect 26: The method of aspect 25, further comprising formulating a new suggested goal by the automated fitness maintenance control location, and downloading the new suggested goal to the fitness and/or nutrition device for review by the of the fitness and/or nutrition participant, if the percentage of goal reached meets a predetermined low at the automated fitness maintenance control location.

Aspect 27: The method of aspect 21, further comprising uploading the second plurality to the automated fitness maintenance control location from the fitness and/or nutrition device in a time frame selected from the group consisting of real-time and after each use.

Aspect 28: The method of aspect 21, wherein choosing the automated fitness and/or nutrition test mode further comprises: comparing of the second plurality to an industry standard fitness maintenance database; and recording of the comparison of the second plurality to an industry standard fitness maintenance database.

Aspect 29: The method of aspect 28, further comprising recording the automated fitness and/or nutrition test mode to an automated fitness and/or nutrition test database, which automated fitness and/or nutrition test database, is accessible to the of the fitness and/or nutrition participant

Aspect 30: The method of aspect 29, further comprising uploading the automated fitness and/or nutrition test database to the automated fitness maintenance control location from the fitness and/or nutrition device in a time frame selected from the group consisting of real-time and after each use.

Aspect 31: The method of aspect 21, wherein said adjusting comprises increasing or decreasing a difficulty level of the use.

Aspect 32: The method of aspect 21, wherein said adjusting comprises varying an environmental factor.

Aspect 33: The method of aspect 32, wherein the environmental factor is at least one selected from the group consisting of music, wind and temperature.

Aspect 34: The method of aspect 21, further comprising providing a fitness and/or nutrition coach that provides interactive information and suggested difficulties of use to the fitness and/or nutrition participant wherein the interactive information and the suggested difficulties are based on comparing by the automated fitness maintenance control location of the first plurality, the second plurality, and the fitness maintenance database.

Aspect 35: The system for assessing fitness and/or nutrition comprising: a protective housing for attachment to a person or a fitness and/or nutrition device or a human or machine-powered vehicle; a GPS sensor configured with the housing for generating earth position signals; a microprocessor configured with the housing for processing the signals to determine weight or at least one of speed and distance traveled of the person or human or machine powered vehicle; and a data unit for reporting one or both of the speed and distance traveled to the person or to a fitness and/or nutrition participant of the human or machine powered vehicle.

Aspect 36: The system of aspect 35, further comprising memory for storing map data from which the microprocessor reports features along a path taken by the person, nutrition device or sport vehicle.

Aspect 37: The system of aspect 35, further comprising memory for storing map data, the processor configured to display, on the data unit, a fitness and/or nutrition participant position using the map data.

Aspect 38: The system of aspect 35, the fitness and/or nutrition device may be comprised as a bike.

Aspect 39: The system of aspect 35, the fitness and/or nutrition device may be comprised as a bicycle or other cycling, running or swimming-like devices.

Aspect 40: The system of aspect 35, further comprising a wireless transmitter configured with the housing for transmitting data indicative of weight or one or both speed and distance traveled to the data unit.

Aspect 41: The system of aspect 40, further comprising a wireless receiver configured with the data unit for receiving the data from the wireless transmitter.

Aspect 42: The system of aspect 41, wherein the data unit comprises a display.

Aspect 43: The system of aspect 42, the data unit comprises a watch, PDA or portable electronic unit.

Aspect 44: The system of aspect 42, further comprising headphones for audibly reporting one or both speed and distance traveled to the person or user.

Aspect 45: The system of aspect 42, wherein the GPS sensor is integrated with clothing to be worn by the person.

Aspect 46: The system of aspect 35, further comprising memory for storing map data, wherein the microprocessor utilizes the map data in determining weight or one or both speed and distance traveled.

Aspect 47: The system of aspect 35, further comprising memory for storing at least one map, the microprocessor configured to identify terrain features near to the person and based on the map.

Aspect 48: The system for assessing fitness and/or nutrition comprising: a protective housing for attachment to a nutrition device, person or to a human-powered sport vehicle; a GPS sensor configured with the housing for generating earth position signals; a microprocessor configured with the housing for processing the signals to determine fitness maintenance data associated with the nutrition device, person or the human or machine powered vehicle; a data unit for reporting the data to the person or to a fitness and/or nutrition participant of the human or machine powered vehicle; a wireless transmitter for wirelessly transmitting the data; and a wireless receiver connected with the data unit for wirelessly receiving the data.

Aspect 49: The system of aspect 48, the microprocessor further configured to determine, from signals of the GPS sensor, one or more of weight, drop distance, airtime, distance traveled, speed and height, as the data.

Aspect 50: The system of aspect 48 further comprises memory for storing map data, wherein the microprocessor utilizes the map data in determining the data.

Aspect 51: The system of aspect 48, further comprising memory for storing at least two maps, the microprocessor configured to identify nutrition value and terrain features near to the person and based on the maps.

Aspect 52: The O'Bay System is a fitness and/or nutrition surface interface method uses on a display device associated with the fitness and/or nutrition device, comprising: connecting the World Fitness Maintenance Network connection to the fitness and/or nutrition device capable of accessing fitness and/or nutrition information stored over the World Fitness Maintenance Network; partitioning the display of the display device to form a second display portion to display one or more controls of the fitness and/or nutrition surface associated with the fitness and/or nutrition device to form a first display portion to display content; and providing a selectable element on the display device that upon selection removes the second display portion from the display.

Aspect 53: The method of aspect 52, further comprising: expanding the first display portion in response to the selection of the selectable element.

Aspect 54: The method of aspect 52, wherein the World Fitness Maintenance Network connection includes access to the Internet.

Aspect 55: The method of aspect 52, wherein the first display portion covers a field portion of the display.

Aspect 56: The method of aspect 52, wherein the second display portion covers a border portion of the display.

Aspect 57: The method of aspect 56, wherein the second display portion occupying the border portion of the display appears to slide like a drawer into the outer edge of the display when removed.

Aspect 58: The fitness and/or nutrition surface interface method improves the usability of an interface for the fitness and/or nutrition device, comprising: connecting the World Fitness Maintenance Network connection to the fitness and/or nutrition exercise device capable of accessing fitness and/or nutrition information stored over the World Fitness Maintenance Network; partitioning a display of a display device associated with the exercise device to form a first display portion to display content; partitioning the display of the display device to form a second display portion to display one or more controls to control the fitness and/or nutrition exercise device surface; and providing a selectable element in the second display portion of the display device that upon selection increases a size of one or more selectable elements in the first display portion of the display device.

Aspect 59: The method of aspect 58, wherein the World Fitness Maintenance Network connection provides access to the Internet.

Aspect 60: The method of aspect 58, wherein the one or more selectable elements in the first portion of the display device capable of increasing in size are hypertext selectable elements.

Aspect 61: The method comprises a computer-readable storage medium tangibly embodying program instructions for use on a display device associated with a fitness and/or nutrition device, the program instructions including instructions operable to cause a processor to: connect the fitness and/or nutrition exercise device to the World Fitness Maintenance Network having access to fitness and/or nutrition information; partition a display of the display device associated with the exercise device to form a first display portion to display content; partition the display of the display device to form a second display portion to display one or more controls to control the fitness and/or nutrition exercise device; and provide a selectable element on the display device that upon selection removes the second display portion from the display.

Aspect 62: The surface interface device comprises a computer-readable storage medium tangibly embodying program instructions for improving the usability of an interface for a fitness and/or nutrition device; the program instructions including instructions operable to cause a processor to: connect the fitness and/or nutrition device to a World Fitness Maintenance Network having access to fitness and/or nutrition information; partition a display of the display device associated with the fitness and/or nutrition exercise device to form a first display portion to display content; partition the display of the display device to form a second display portion to display one or more controls to control the fitness and/or nutrition device; and provide a selectable element in the second display portion of the display device that upon selection increases a size of one or more selectable elements in the first display portion of the display device.

Aspect 63: The device of aspect 62, further comprising program instructions operable to cause a processor to expand the first display portion in response to the selection of the selectable element.

Aspect 64: The device of aspect 62, wherein the World Fitness Maintenance Network has access to the Internet.

Aspect 65: The device of aspect 62, wherein the first display portion covers a field portion of the display.

Aspect 66: The device of aspect 62, wherein the second display portion covers a border portion of the display.

Aspect 67: The device of aspect 66, wherein the second display portion occupying the border portion of the display appears to slide like a drawer into the outer edge of the display when removed.

Aspect 68: The device of aspect 62, wherein the World Fitness Maintenance Network has access to the Internet.

Aspect 69: The device of aspect 62, wherein the selectable elements in the first display portion of the display device capable of increasing in size are hypertext selectable elements.

Aspect 70: The O'Bay System is also a fitness and/or nutrition method including the steps of: A. providing a processor or an artificial intelligence system with a protocol generating algorithm; B. providing a fitness and/or nutrition device in communication with a processor or an artificial intelligence system; C. inputting user data into said processor or an artificial intelligence system; D. generating fitness and/or nutrition protocol according to said protocol generating algorithm and said user data; E. reviewing and allowing for modification of said fitness and/or nutrition protocol; F. transferring said fitness and/or nutrition protocol to said fitness and/or nutrition device; G. performing a fitness and/or nutrition session on said fitness and/or nutrition device; H. generating information regarding said fitness and/or nutrition session; I. transferring said information to said processor or an artificial intelligence system; and J. reviewing said fitness and/or nutrition information.

Aspect 71: The method as described in aspect 70, further comprising: K. modifying said protocol, thus generating a modified protocol; L. reviewing said modified protocol; and M. transferring said modified protocol to said fitness and/or nutrition device.

Aspect 72: The method as described in aspect 71, further comprises: N. providing predetermined fitness and/or nutrition boundaries to values of said information; O. executing an alert when a value of said fitness and/or nutrition information is not within said boundaries.

Aspect 73: The method as described in aspect 72, wherein the step of executing an alert is in the form of a message sent to the clinician.

Aspect 74: The method as described in aspect 72, wherein the step of executing an alert is in the form of a message sent to the fitness and/or nutrition participant.

Aspect 75: The method as described in aspect 74, wherein said message is in the form of a visual message displayed on a message line.

Aspect 76: The method as described in aspect 74, wherein said message is in the form of an audible message.

Aspect 77: The method as described in aspect 70, further comprising: K. providing predetermined boundaries to values of said information; L. executing an alert when a value of said information is not within said boundaries.

Aspect 78: The method as described in aspect 77, wherein the step of executing an alert is in the form of a message sent to the clinician.

Aspect 79: The method as described in aspect 77, wherein the step of executing an alert is in the form of a message sent to the fitness and/or nutrition participant.

Aspect 80: The method as described in aspect 79, wherein said message is in the form of a visual message displayed on a message line.

Aspect 81: The method as described in aspect 79, wherein said message is in the form of an audible message.

Aspect 82: The method as described in aspect 70, wherein said data is data selected from the group consisting of age, height, sensor, sex and special considerations.

Aspect 83: The method as described in aspect 82, wherein said special considerations are physical conditions.

Aspect 84: The method as described in aspect 83, wherein said physical conditions are conditions selected from the group consisting of low VO.sub.2max, high blood pressure, obesity, osteoporosis and joint injuries.

Aspect 85: The method as described in aspect 70, wherein step (F) is further comprised of transferring control codes to say fitness and/or nutrition device, the control codes enabling a determined fitness and/or nutrition performance of said fitness and/or nutrition device.

Aspect 86: The method as described in aspect 70, wherein said information is data selected from the group consisting of fitness and/or nutrition data and physiological data.

Aspect 87: The method as described in aspect 84, wherein said data is data selected from the group consisting of machine power output, total work done, speed, force, range of motion and relative perceived exertion.

Aspect 88: The method as described in aspect 84, wherein said physiological data is data selected from the group consisting of blood pressure values and heart rate values.

Aspect 89: The method as described in aspect 70, wherein said communication of step (B) is made by a method of communication selected from the group consisting of a LAN system, and internet system and an intranet system.

Aspect 90: The VirtuFit Chamber is a virtual reality fitness simulator system comprising: a tracking, over time and/or distance and/or calorie consumption and/or weight, system for continuously tracking the overall physical location of a virtual reality fitness participant in a defined physical space; a nationally certified exercise physiologist and dietician/nutritionist or computer operatively coupled to the tracking, over time and/or distance and/or calorie consumption and/or weight, system for updating in real time a virtual reality fitness participants location in a virtual space corresponding to the physical location of the virtual reality fitness participant in the physical space, and for updating a view of the virtual space; and a display operatively coupled to the computer, wherein the display displays the view of the virtual space; wherein the view is from a point of view in the virtual space corresponding to a location on a line directed outward from the display into the physical space.

Aspect 91: The system of aspect 90, wherein the view of the virtual space includes a virtual representation of at least a part of the virtual reality fitness participant.

Aspect 92: The system of aspect 91, wherein the view is from a point of view in the virtual space behind the virtual representation of the at least part of the virtual reality fitness participant.

Aspect 93: The system of aspect 90, wherein the view of the virtual space is a first-person perspective view from the virtual reality fitness participant location.

Aspect 94: The system of aspect 90, wherein the nationally certified exercise physiologist and dietician/nutritionist or computer provides at least one indicium of performance of the virtual reality fitness participant moving in the physical space, wherein the at least one indicium is or is derived from a measure of a movement parameter of the virtual reality fitness participant.

Aspect 95: The system of aspect 94, wherein the view of the virtual space includes a virtual reality fitness participant icon located at the virtual reality fitness participant location.

Aspect 96: The system of aspect 95, wherein the nationally certified exercise physiologist and dietician/nutritionist or computer updates a protagonist virtual location of a virtual protagonist in the virtual space, and the view includes a protagonist icon located at the protagonist virtual location.

Aspect 97: The system of aspect 96, wherein the nationally certified exercise physiologist and dietician/nutritionist or computer updates the protagonist virtual location as a function of a selectable modulation factor.

Aspect 98: The system of aspect 97, wherein the modulation factor controls the rate of change of the protagonist virtual location.

Aspect 99: The system of aspect 96, wherein the updating of the protagonist virtual location is made in response to the changes in the physical location of the virtual reality fitness participant, such that the virtual protagonist and another virtual reality fitness participant engage in an interactive task.

Aspect 100: The system of aspect 99, wherein the interactive task involves the virtual reality fitness participant attempting to create an asynchronous event, and the at least one indicium of performance of the virtual reality fitness participant includes a measure of the virtual reality fitness participant's ability to maximize spatial differences over time between the virtual reality fitness participant and the virtual protagonist.

Aspect 101: The system of aspect 96, wherein said at least one indicium of performance of the virtual reality fitness participant includes a measure of the virtual reality fitness participant's ability over a time interval to minimize spatial differences between the virtual reality fitness participant virtual location and the protagonist virtual location.

Aspect 102: The system of aspect 101, wherein the at least one indicium of a performance of the virtual reality fitness participant includes a time in compliance.

Aspect 103: The system of aspect 98, wherein the at least one indicium of performance of the virtual reality fitness participant includes a measure of the virtual reality fitness participants bi-lateral vector accelerations and decelerations.

Aspect 104: The system of aspect 98, wherein the at least one indicium of performance of the virtual reality fitness participant includes a measure of the virtual reality fitness participants power.

Aspect 105: The simulator system comprising: a tracking, over time and/or distance and/or calorie consumption and/or weight, system for continuously tracking the overall physical location of a virtual reality fitness participant in a defined physical space; a nationally certified exercise physiologist and dietician/nutritionist or computer operatively coupled to the tracking, over time and/or distance and/or calorie consumption and/or weight, system for updating in real time a virtual reality fitness participant virtual location in a virtual space corresponding to the physical location of the virtual reality fitness participant in the physical space, and for updating a view of the virtual space; and a display operatively coupled to the computer, wherein the display displays the view of the virtual space; wherein the view is from a point of view in the virtual space corresponding to a location on a line directed substantially perpendicular to the display.

Aspect 106: The system of aspect 105, wherein the view of the, virtual space includes a virtual representation of at least a part of the virtual reality fitness participant.

Aspect 107: The system of aspect 106, wherein the view is from a point of view in the virtual space behind the virtual representation of the at least part of the virtual reality fitness participant.

Aspect 108: The system of aspect 105, wherein the view of the virtual space is a first-person perspective view from the virtual reality fitness participant location.

Aspect 109: The system of aspect 105, wherein the nationally certified exercise physiologist and dietician/nutritionist or computer provides at least one indicium of performance of the virtual reality fitness participant moving in the physical space, wherein the at least one indicium is or is derived from a measure of a movement parameter of the virtual reality fitness participant.

Aspect 110: The Triathlon and Nutrition Periodization Application System is a peer-to-peer, triathlon and nutrition periodization information application system that comprises: a triathlon and nutrition periodization terminal and at least one triathlon and nutrition periodization terminal, which is communicable with the triathlon and nutrition periodization terminal through the World Fitness Maintenance Network; the triathlon and nutrition periodization terminal comprising a triathlon and nutrition periodization peer-to-peer application device that is manually operable by a participant to generate a sample triathlon and nutrition periodization performance; a triathlon and nutrition periodization processor device or an artificial intelligence system that provisionally prepares triathlon and nutrition periodization test piece in the at least one terminal; compare the triathlon and nutrition periodization test piece with the sample triathlon and nutrition periodization performance to determine differences there between to detect a triathlon and nutrition periodization level of the triathlon and nutrition periodization participant; and formulates a triathlon and nutrition periodization curriculum corresponding to the triathlon and nutrition periodization level; a display device that is controlled by the processor device or an artificial intelligence system to display a triathlon and nutrition periodization lesson and score according to the triathlon and nutrition periodization curriculum so that the triathlon and nutrition periodization lesson and score can be rendered by operating the peer-to-peer application device to generate the sample triathlon and nutrition periodization performance; the triathlon and nutrition periodization personnel terminal comprising a receiver device that receives data representative of the sample triathlon and nutrition periodization performance from the terminal through the World Fitness Maintenance Network; a triathlon and nutrition periodization processor device or an artificial intelligence system that is operated by an exercise physiologist and dietician/nutritionist or certified personal trainer to evaluate the sample triathlon and nutrition periodization performance according to the received data so as to arrange triathlon and nutrition periodization advice, which could not be arranged by the terminal; and a transmitter device that transmits back the triathlon and nutrition periodization advice to the terminal, wherein the terminal, upon receiving the triathlon and nutrition periodization advice from the exercise physiologist and dietician/nutritionist or certified personal trainer terminal through the World Fitness Maintenance Network; controls the display device to display a triathlon and nutrition periodization lesson and score and a comment on the exercise part based on the triathlon and nutrition periodization advice, thereby prompting the triathlon and nutrition periodization participant to exercise according to the comment.

Aspect 111: The triathlon and nutrition periodization peer-to-peer application device comprises: communicable with the center through the World Fitness Maintenance Network, a triathlon and nutrition periodization peer-to-peer application device that is manually operable by a trainee to generate a sample triathlon and nutrition periodization performance; a processor device or an artificial intelligence system that provisionally prepares a triathlon and nutrition periodization test piece in the trainee's terminal, compares the triathlon and nutrition periodization test piece with the sample triathlon and nutrition periodization performance to determine differences there between to detect a triathlon and nutrition periodization level of the trainee, and formulates a triathlon and nutrition periodization curriculum corresponding to the triathlon and nutrition periodization level; a display device that is controlled by the processor device or an artificial intelligence system to display a triathlon and nutrition periodization lesson and score according to the triathlon and nutrition periodization curriculum so that the triathlon and nutrition periodization lesson and score can be rendered by operating the peer-to-peer information application triathlon and nutrition periodization device to generate the sample triathlon and nutrition periodization performance; a transmitter device that transmits data representative of the sample triathlon and nutrition periodization performance to the center through the World Fitness Maintenance Network so that the center evaluates the sample triathlon and nutrition periodization performance according to the data to arrange triathlon and nutrition periodization advice, which could not be arranged by the local device; and a receiver device that receives the triathlon and nutrition periodization advice from the center through the World Fitness Maintenance Network; wherein the triathlon and nutrition periodization peer-to-peer application device operates upon receiving the triathlon and nutrition periodization advice for controlling the display device to display an exercise part of the triathlon and nutrition periodization lesson and score and a comment on the exercise part based on the triathlon and nutrition periodization advice, thereby prompting the triathlon and nutrition periodization participant to exercise part according to the triathlon and nutrition periodization comment.

Aspect 112: The device as described in aspect 111, wherein the triathlon and nutrition periodization processor device or an artificial intelligence system compares the triathlon and nutrition periodization performance data representative of the sample triathlon and nutrition periodization performance generated by the triathlon and nutrition periodization peer-to-peer application device with triathlon and nutrition periodization data representative of the triathlon and nutrition periodization lesson and score displayed on the display device; and operates if a difference between the sample triathlon and nutrition periodization performance and the triathlon and nutrition periodization lesson and score exceeds a triathlon and nutrition periodization acceptable degree for performing an exercise part of the triathlon and nutrition periodization score; such that the exercise part is emphasized to facilitate triathlon and nutrition periodization practicing on the triathlon and nutrition periodization device.

Aspect 113: The device as described in aspect 111, further comprising an audio and visual source device that is controlled by the processor device or an artificial intelligence system according to the triathlon and nutrition periodization advice for generating a model triathlon and nutrition periodization performance corresponding to the exercise part of the triathlon and nutrition periodization score; such that the model triathlon and nutrition periodization performance is modified according to the triathlon and nutrition periodization comment to audibly and visually teach the trainee.

Aspect 114: The central triathlon and nutrition periodization peer-to-peer application device comprises: communicable through the World Fitness Maintenance Network with a local training device having peer-to-peer information application triathlon and nutrition periodization device manually operable by the trainee to generate a sample triathlon and nutrition periodization performance, a processor device or an artificial intelligence system for provisionally preparing a triathlon and nutrition periodization test piece in the trainee's terminal, comparing the triathlon and nutrition periodization test piece with the sample triathlon and nutrition periodization performance to determine differences there between to detect the triathlon and nutrition periodization level of the trainee, and formulating a triathlon and nutrition periodization curriculum corresponding to the triathlon and nutrition periodization level, and a display device controlled by the processor device or an artificial intelligence system to display a triathlon and nutrition periodization lesson and score according to the triathlon and nutrition periodization curriculum so that the triathlon and nutrition periodization lesson and score can be rendered by operating the triathlon and nutrition periodization device to generate the sample triathlon and nutrition periodization performance, the central training device comprising: a receiver device that receives data representative of the sample triathlon and nutrition periodization performance from the local training device through the World Fitness Maintenance Network; a processor device or an artificial intelligence system that is operated by a personal exercise physiologist and dietician/nutritionist or certified personal trainer to evaluate the sample triathlon and nutrition periodization performance according to the received data so as to arrange triathlon and nutrition periodization advice, which could not be arranged by the local training device; and a transmitter device that transmits back the triathlon and nutrition periodization advice to the local training device, thereby enabling the local training device to display an exercise part of the triathlon and nutrition periodization lesson and score and a comment on the exercise part for prompting the trainee to play the exercise part according to the comment.

Aspect 115: The triathlon and nutrition periodization peer-to-peer application device comprises: communicable with an supervisory computer through the World Fitness Maintenance Network for remotely training matching with a skill level; triathlon and nutrition periodization device means manually operable by the user's exercise physiologist and dietician/nutritionist or certified personal trainer for generating a triathlon and nutrition periodization performance; processor means for provisionally preparing a triathlon and nutrition periodization test piece in an exercise physiologist and dietician/nutritionist or certified personal trainer's terminal, comparing the triathlon and nutrition periodization test piece with the triathlon and nutrition periodization performance generated by the triathlon and nutrition periodization device means to determine differences there between to detect the triathlon and nutrition periodization level of the user and formulating a triathlon and nutrition periodization curriculum corresponding to the triathlon and nutrition periodization level of the user; display means for displaying a triathlon and nutrition periodization lesson and score in matching with the triathlon and nutrition periodization skill level of the user according to the curriculum so that the user's exercise physiologist and dietician/nutritionist or certified personal trainer is prompted to render the displayed triathlon and nutrition periodization lesson and score by operating the triathlon and nutrition periodization device means for generating a sample triathlon and nutrition periodization performance; wherein processor means compares triathlon and nutrition periodization event data representative of the sample triathlon and nutrition periodization performance with note triathlon and nutrition periodization performance data representative of the triathlon and nutrition periodization lesson and score to locally execute a quantitative and qualitative evaluation of the sample triathlon and nutrition periodization performance; transmitter means for transmitting the triathlon and nutrition periodization event data representative of the sample triathlon and nutrition periodization performance to the supervisory computer through the World Fitness Maintenance Network so that the supervisory computer can work to remotely provide a qualitative evaluation of the sample triathlon and nutrition periodization performance according to the event data to thereby arrange instruction data, the qualitative evaluation being different from the quantitative triathlon and nutrition periodization performance evaluation; and being locally unavailable; and receiver means for receiving the triathlon and nutrition periodization instruction data from the supervisory computer through the World Fitness Maintenance Network to pass the triathlon and nutrition periodization instruction data to the processor means; wherein the processor means operates based on either of the local quantitative triathlon and nutrition periodization performance evaluation; and the remote qualitative evaluation represented by the received instruction data for modifying the triathlon and nutrition periodization lesson and score effective to improve the triathlon and nutrition periodization skill level of the user.

Aspect 116: The device as described in aspect 115, wherein the processor means includes means operative based on the triathlon and nutrition periodization measurements instruction data for controlling the display means to display an exercise part of the triathlon and nutrition periodization measurements lesson and score and a comment on the exercise part, thereby prompting the triathlon and nutrition periodization measurements user's exercise physiologist and dietician/nutritionist or certified personal trainer to play the exercise part according to the comment.

Aspect 117: The device as described in aspect 116, further comprising sound source means controlled by the processor means according to the triathlon and nutrition periodization instruction data for generating a model triathlon and nutrition periodization performance corresponding to the exercise part of the triathlon and nutrition periodization lesson and score such that the model triathlon and nutrition periodization performance is modified according to the comment to audibly teach the user's exercise physiologist and dietician/nutritionist or certified personal trainer.

Aspect 118: The device as described in aspect 115, wherein the processor means includes means operative when the quantitative triathlon and nutrition periodization performance evaluation indicates that a difference between the sample triathlon and nutrition periodization performance and the triathlon and nutrition periodization lesson and score exceeds a triathlon and nutrition periodization acceptable degree for arranging an exercise part of the triathlon and nutrition periodization lesson and score such that the exercise part is rhythmically emphasized to facilitate operation of the triathlon and nutrition periodization device means.

Aspect 119: The device as described in aspect 115, wherein the processor means comprises means for executing the quantitative triathlon and nutrition periodization performance evaluation in terms of at least one of a rhythm, a pitch and a volume of the sample triathlon and nutrition periodization measurements performance while the qualitative evaluation is executed in terms of a total rendition of the sample triathlon and nutrition periodization measurements performance.

Aspect 120: The device as described in aspect 115, wherein the processor means includes means for analyzing an initial triathlon and nutrition periodization measurements performance generated by the triathlon and nutrition periodization measurements user to determine the triathlon and nutrition periodization skill level of the measurements user, and means for formulating the triathlon and nutrition periodization measurements lesson and score in matching with the triathlon and nutrition periodization skill level.

Aspect 121: The method of using a triathlon and nutrition periodization peer-to-peer application device comprises; a display and a triathlon and nutrition periodization device manually operable by a user's exercise physiologist and dietician/nutritionist or certified personal trainer for generating a triathlon and nutrition periodization performance and being communicable with a supervisory computer through the World Fitness Maintenance Network for remotely training the user's exercise physiologist and dietician/nutritionist or certified personal trainer in matching with a skill level, the method comprising the steps of: provisionally preparing a triathlon and nutrition periodization test piece in an exercise physiologist and dietician/nutritionist or certified personal trainer's terminal; comparing the triathlon and nutrition periodization test piece with a triathlon and nutrition periodization performance generated by the triathlon and nutrition periodization device to determine differences there between in order to detect the triathlon and nutrition periodization level of the user; formulating a triathlon and nutrition periodization curriculum corresponding to the triathlon and nutrition periodization level of the user; displaying a triathlon and nutrition periodization lesson and score on the display in matching with the triathlon and nutrition periodization skill level of the user according to the curriculum so that the user's exercise physiologist and dietician/nutritionist or certified personal trainer is prompted to render the displayed triathlon and nutrition periodization lesson and score by operating the triathlon and nutrition periodization device for generating a sample triathlon and nutrition periodization performance; comparing triathlon and nutrition periodization event data representative of the sample triathlon and nutrition periodization performance with noted triathlon and nutrition periodization performance data representative of the triathlon and nutrition periodization lesson and score to execute a quantitative and qualitative evaluation of the sample triathlon and nutrition periodization performance; transmitting the triathlon and nutrition periodization event data representative of the sample triathlon and nutrition periodization performance to the supervisory computer through the World Fitness Maintenance Network so that the supervisory computer can work to provide a qualitative evaluation of the sample triathlon and nutrition periodization performance according to the event data to thereby arrange instruction data, the qualitative being different from the quantitative triathlon and nutrition periodization performance evaluation and being unavailable by the music device; receiving the triathlon and nutrition periodization instruction data from the supervisory computer through the World Fitness Maintenance Network; and modifying the triathlon and nutrition periodization lesson and score in order to improve the triathlon and nutrition periodization skill level of the user based on either of the quantitative triathlon and nutrition periodization performance evaluation and the qualitative evaluation represented by the received instruction data.

Aspect 122: A machine readable medium for use in a triathlon and nutrition periodization peer-to-peer application device comprises; having a cpu, a display and a triathlon and nutrition periodization device manually operable by a user's exercise physiologist and dietician/nutritionist or certified personal trainer to generate a triathlon and nutrition periodization performance and being communicable with a supervisory computer through the World Fitness Maintenance Network for remotely training the user's exercise physiologist and dietician/nutritionist or certified personal trainer in matching with a skill level, the medium containing a program executable by the cpu for causing the music device to perform the method comprising the steps of: provisionally preparing a triathlon and nutrition periodization test piece in an exercise physiologist and dietician/nutritionist or certified personal trainer's terminal; comparing the triathlon and nutrition periodization test piece with a triathlon and nutrition periodization performance generated by the triathlon and nutrition periodization device to determine differences there between in order to detect the triathlon and nutrition periodization level of the user; formulating a triathlon and nutrition periodization curriculum corresponding to the triathlon and nutrition periodization level of the user; displaying a triathlon and nutrition periodization lesson and score on the display in matching with the triathlon and nutrition periodization skill level of the user according to the curriculum so that the user's exercise physiologist and dietician/nutritionist or certified personal trainer is prompted to render the displayed triathlon and nutrition periodization lesson and score by operating the triathlon and nutrition periodization device for generating a sample triathlon and nutrition periodization performance; comparing triathlon and nutrition periodization event data representative of the sample triathlon and nutrition periodization performance with note triathlon and nutrition periodization performance data representative of the triathlon and nutrition periodization lesson and score to execute a quantitative and qualitative evaluation of the sample triathlon and nutrition periodization performance; transmitting the triathlon and nutrition periodization event data representative of the sample triathlon and nutrition periodization performance to the supervisory computer through the World Fitness Maintenance Network so that the supervisory computer can work to provide a qualitative evaluation of the sample triathlon and nutrition periodization performance according to the triathlon and nutrition periodization event data to thereby arrange instruction data, the qualitative evaluation being different from the quantitative triathlon and nutrition periodization performance evaluation and being unavailable by the music device; receiving the triathlon and nutrition periodization instruction data from the supervisory computer through the World Fitness Maintenance Network; and modifying the triathlon and nutrition periodization lesson and score in order to improve the triathlon and nutrition periodization skill level of the user based on either of the quantitative triathlon and nutrition periodization performance evaluation and the qualitative evaluation represented by the received triathlon and nutrition periodization instruction data.

Aspect 123: The World Nutrition Metric System is administered through our nutrition and metric system comprised solely of the oral administration and consumptions of nutritionally therapeutic amount of macronutrient-based blood type-based kosher and organic food and drink under the Nationally Recommended Dietary Allowance standard and scale of the country of operation.

Aspect 124: The method of aspect 123 also comprises the therapeutically effective behavior of fasting from the oral administration of individually toxic, man-made, genetically-engineered or altered food products category to increase body metabolism and improve waste elimination.

Aspect 125: The methods of aspects 123 or 124, wherein said nutrition effective and said therapeutically effective amounts recommended by strict medical, exercise or nutrition guidelines, direction or supervision will also be known as the World Nutrition Metric Allowance Standard and Scale™.

Aspect 126: A method for electronically recording, calculating and monitoring of macronutrient-based blood type-based kosher and organic foods and drinks nutritional consumptions information includes providing computer means with memory for storing nutritional and product identification information of macronutrient-based blood type-based kosher and organic foods and drinks available and enables said computer means to process said nutritional metric information and said product identification information.

Aspect 127: The method for administering the World Fitness Maintenance Metric Standard and Scale™. The World Fitness Maintenance Metric Standard and Scale™ will consist of assessing, measuring, quantifying, evaluating, validating, correlating, comparing, interpreting, ranking and tracking, over time and/or distance and/or calorie consumption and/or weight, incremental changes in peak, health, wellness, overweight and obesity individuals through World Fitness Maintenance Metric values and information like, but not limited to, VO2 max test, lactate threshold analysis, metabolic rate analysis, swim-stroke force analysis, dry-land strength test, spin scan, motion analysis and body composition measurements before, during and after fitness maintenance metric performances judged by distance and/or time.

In the advanced stage of O'Bay System, the exclusive, therapeutic use of macronutrient-based consumption of blood-type specific kosher and organic food and drink will be used upon specific groups of World Fitness Maintenance Metric and health-related individuals to monitor and generate fitness maintenance values. Overall evaluations will also include, but not limited to, VO2 max test, lactate threshold analysis, metabolic rate analysis, swim-stroke force analysis, dry-land strength test, spin scan, motion analysis and body composition. Research documentation and tracking, over time and/or distance and/or calorie consumption and/or weight, will be done through laboratory analyzes levels of total peak, health, wellness, overweight and obesity fitness and overall associated fitness maintenance and health conditions as they pertain to fitness maintenance metric performance.

Along with the World Fitness Maintenance Metric risks in order to predict, improve and forecast future individual and societal gains, World Fitness Maintenance Metric laboratory results will analyze levels of total World Fitness Maintenance Metric performance, optimal sports medicine and exercise information along with an overall associated fitness maintenance and health-related risks based upon specific sets or groups of pre-determined pertinent, validated and cross-correlatable categories, parameters and variables of said strength, conditioning, power, speed, quickness and response time levels. This will include the weighing and adjusting pre-determined optimal ranges of peak, health, wellness, overweight and obesity sports medicine and exercise science values, the fitness, nutrition, and health-related fitness maintenance metric participants to said fitness maintenance metric levels; a standard means and standard deviation values of said method and preparing for individuals and groups using information gathered from at least two of a series of assessed; a measured and clinically will evaluate on-site and mobile laboratory tests and measurements of at least one of individuals and groups and a scientific total peak, health, wellness, overweight and obesity fitness in the fitness maintenance regiment and against an associated fitness maintenance and health risks database.

Aspect 128: The method of aspect 127 comprising the following steps of: establishing a list of measurable, quantifiable, correlatable, comparable, validatable, and trackable categories, parameters and variables of said fitness maintenance response time levels along with total peak, health, wellness, overweight and obesity fitness maintenance metric values and any overall associated fitness and nutrition periodization, medical and health risk-related information; obtaining fitness maintenance response time data from individuals and groups corresponding to specific predetermined sets or groups of pertinent, validated and correlatable categories, parameters and variables of total fitness maintenance metric optimal health, and any overall associated medical and health-related risks; creating a data gathering scheme for each said category, parameter, and variable, and combination of said peak, health, wellness, overweight and obesity fitness maintenance metric categories, parameters, and variables; utilizing the scheme to gather both subjective and objective data on individuals, and groups; quantifying and compiling the data, checking the data for validity, correlating, comparing, updating and storing the quantified, compiled, validated, correlated, compared, updated and stored data as an inventory of total peak, health, wellness, overweight and obesity fitness maintenance metric data and any associated fitness maintenance metric risk-related knowledge; locally weighting and adjusting the pre-determined optimal ranges of peak, health, wellness, overweight and obesity fitness maintenance metric totals, and overall associated health and fitness maintenance metric risks as well as the standard means and the standard deviation values of said method; and, preparing an evaluation of said knowledge by correlating and comparing said knowledge with: (1) ideal predetermined rankings of total peak, health, wellness, overweight and obesity fitness maintenance metric category data and associated health and fitness-related risks; (2) said scientific total peak, health, wellness, overweight and obesity fitness maintenance metric data and any associated health and fitness-related risks in the overall fitness maintenance metric database; and (3) any previously scientific total peak, health, wellness, overweight and obesity fitness maintenance metric data and any associated health, fitness maintenance metric risks assessed and measured, and laboratory analyzed individuals and groups to create a report having two parts, a first part having a series of fitness maintenance metric analysis and information known as the World Fitness Maintenance Metric Scale™. The World Fitness Maintenance Metric Scale™ will be categorized as follows:
Excellent Fitness Maintenance Metric results in an extremely low cardiovascular disease and health risk through a combination of cardiovascular/respiratory, endurance, stamina, strength, flexibility, power, coordination, agility, balance and accuracy exercises with professional or excellent nutrition periodization guidelines adherence.
Good Fitness Maintenance Metric results in a low cardiovascular disease and health risk through a combination of cardiovascular/respiratory, endurance, stamina, strength, flexibility, power, coordination, agility, balance and accuracy exercises with an above average or good nutrition periodization guidelines adherence.
Average Fitness Maintenance Metric results in an average cardiovascular disease and health risk through a combination of cardiovascular/respiratory, endurance, stamina, strength, flexibility, power, coordination, agility, balance and accuracy exercises, with self-guided or average nutrition periodization guidelines adherence.
Poor Fitness Maintenance Metric results in a high probability of cardiovascular disease and health risk through a limited amount of cardiovascular/respiratory, endurance, stamina, strength, flexibility, power, coordination, agility, balance and accuracy exercises, with below average nutrition periodization guidelines.
Dangerous Fitness Maintenance Metric results in an extremely high cardiovascular disease and health risk through a nonexistent combination of cardiovascular/respiratory, endurance, stamina, strength, flexibility, power, coordination, agility, balance and accuracy exercises, with no nutrition periodization guidelines or boundaries.

The second part on the World Fitness Maintenance Metric Scale™ comprising a series of individualized testing, evaluations and recommendations for fitness maintenance metric improvement known as the World Fitness Maintenance Metric Standard™ each of said second part being printed proximate a selected one of said first part for review in combination with said one of said first part.

Aspect 129: The method of the World Fitness Maintenance Metric Standard™ includes preparing an Individualized Fitness Maintenance Plan for fitness maintenance metric participant using the O'Bay Quotient™ (OQ™) format from cardiovascular/respiratory, endurance, stamina, strength, flexibility, power, coordination, agility, balance and accuracy information gathering, evaluation and recommendations to fitness maintenance metric participant, along with any previously created record in the fitness maintenance metric database records. The method comprising the steps of

a. establishing a list of measurable categories of cardiovascular/respiratory, endurance, stamina, strength, flexibility, power, coordination, agility, balance and accuracy for peak, health, wellness, overweight and obesity fitness related to fitness maintenance metric information,

b. creating a data gathering scheme for each said category,

c. utilizing the scheme to gather subjective and objective data on the fitness maintenance metric participant,

d. compiling the data, checking the data for validity, and storing the validated data as an inventory of peak, health, wellness, overweight and obesity fitness, nutrition and risk-related knowledge, and

e. preparing an evaluation of said knowledge by comparing said knowledge with said fitness maintenance metric database to create a report having two parts, a first part having a series of visual depictions each having analysis information categorized as:

i. Excellent O'Baymetrics known as extremely low risk,

ii. Good O'Baymetrics known as low risk,

iii. Average O'Baymetrics known as average risk

iv. Poor O'Baymetrics known as high risk

v. Dangerous O'Baymetrics also known as extremely high risk and a second part comprising a series individualized, O'Bay Quotient Analysis™ (OQA™) evaluations and recommendations for O'Baymetrics improvement, each of said second part being printed proximate a selected one of said first part for review in combination with said one of said first part.

Aspect 130: The method per aspect 129 including establishing one of said categories as measured quantifiable parameters comprising

i. swimming measurements may include endurance sets, force sets, speed sets, muscular-endurance sets, anaerobic-endurance sets, power sets and swim test workouts;

ii. running measurements, may include endurance sets, force sets, speed sets, muscular-endurance sets, anaerobic-endurance sets, power sets and running test workouts;

iii. cycling measurements, may include endurance sets, force sets, speed sets, muscular-endurance sets, anaerobic-endurance sets, power sets and cycling test workouts;

iv. weightlifting measurements, may include endurance sets, force sets, speed sets, muscular-endurance sets, anaerobic-endurance sets, power sets and weightlifting test workouts;

v. nutrition periodization measurements, may include the consumption, digestive and utilization patterns.

All said categories comprising physical parameters of scientific peak, health, wellness, overweight and obesity O'Baymetrics values and health risk.

Aspect 131: The method per aspect 129 including establishing one of said categories as measured quantifiable parameters comprising

i. cardiopulmonary measurements, including heart rate, stroke volume, cardiac output, blood pressure, oxygen consumption, total peripheral resistance, arteriovenous oxygen difference measurements;

ii. Ventilatory measurements, including breathing frequency, tidal volume and ventilation;

iii. Body composition measurements, including muscle fiber analysis, length-tension relationship, and force-velocity relationship;

iv. Muscular strength and fatigue measurements through short-duration, high intensity and endurance exercise by monitoring values like glycogen depletion;

v. stress/distress;

vi. Anthropometric measurements, including standing height, body weight, girths, bone widths, height and weight ratio, and skin fold measurements;

vii. Range of motion measurements, including Bony, ligament and muscular limitations,

viii. Biomechanical measurements, including center of balance and gravity, torque;

ix. Energy expenditure measurements during biomechanical exercises;

x. Flexibility measurements; and

xi. Additional diagnostic tests for special population.

All said categories comprising objective physiological measurements of scientific peak, health, wellness, overweight and obesity fitness, nutrition and health risk.

Aspect 132: The method per aspect 129 including establishing one of said categories as analyzed quantifiable parameters comprising

i. blood chemistries

ii. Urine,

iii. Lipids, and

iv. Immune system,

All said categories comprising objective laboratory parameters of scientific peak, health, wellness, overweight and obesity fitness, nutrition and health risk.

Aspect 133: The method per aspect 129 including in method step “e” preparing said evaluation in the form of at least one visually accented graph, each graph having an associated report.

Aspect 134: The method per aspect 133 in which said graph has a graduated 10-point metric scale.

Aspect 135: The method per aspect 134 in which each two points of said 10-point metric scale depict one of said visual depictions.

Aspect 136: A method for assessing, measuring, quantifying, evaluating, validating, correlating, comparing, interpreting, ranking and tracking, over time and/or distance and/or calorie consumption and/or weight, small incremental changes in at least one fitness maintenance metric participants and a group's assessed, and measured, and laboratory analyzed levels of total peak, health, wellness, overweight and obesity fitness, nutrition and overall associated health risks based on specific sets or groups of pre-determined pertinent, validated and cross-correlatable categories, parameters and variables of said levels, and weighing and adjusting pre-determined optimal ranges of said levels as well as standard means and standard deviation values of said method and preparing personal analyses for fitness maintenance metric participants and groups using information gathered from at least two of a series of assessed, and measured and clinically evaluated on-site and mobile laboratory tests and measurements of at least one fitness maintenance metric participants and groups and a scientific total peak, health, wellness, overweight and obesity fitness, nutrition and associated medical health risks and fitness maintenance metric database, the method comprising the steps of:

a. establishing a list of measurable, quantifiable, correlatable, comparable, validatable, and trackable categories, parameters and variables of said levels of total peak, health, wellness, overweight and obesity fitness, nutrition and any overall associated health and fitness risk-related information;

b. obtaining testing data from an O'Baymetrics participants and groups corresponding to specific predetermined sets or groups of pertinent, validated and correlatable categories, parameters and variables of total peak, health, wellness, overweight and obesity fitness, nutrition and any overall associated fitness, nutrition, and health risks;

c. creating a data gathering scheme for each said category, parameter, and variable, and combination of said categories, parameters, and variables;

d. utilizing the scheme to gather both subjective and objective data on O'Baymetrics participants, and groups;

e. quantifying and compiling the data, checking the data for validity, correlating, comparing, updating and storing the quantified, compiled, validated, correlated, compared, updated and stored data as an inventory of total peak, health, wellness, overweight and obesity fitness, nutrition and any associated health risk-related knowledge;

f. locally weighting and adjusting the pre-determined optimal ranges of total peak, health, wellness, overweight and obesity fitness, nutrition and overall associated fitness, nutrition, and health risks as well as the standard means and the standard deviation values of said method; and,

g. preparing an evaluation of said knowledge by correlating and comparing said knowledge with: (1) ideal predetermined rankings of total peak, health, wellness, overweight and obesity fitness, nutrition and associated health risks; (2) said scientific total peak, health, wellness, overweight and obesity fitness, nutrition and any associated health risks within the fitness maintenance database; and (3) any previously scientific total peak, health, wellness, overweight and obesity fitness, nutrition and any associated health risks assessed and measured, and laboratory analyzed fitness maintenance metric participants and groups to create a report having two parts, a first part having a series of visual depictions each having analysis information categorized as:

i. Excellent O'Baymetrics also known as extremely low risk,

ii. Good O'Baymetrics also known as low risk,

iii. Average O'Baymetrics also known as average risk,

iv. Poor O'Baymetrics also known as high risk,

v. Dangerous O'Baymetrics also known as extremely high risk, and a second part comprising a series individualized evaluations and recommendations for improvement, each of said second part being printed proximate a selected one of said first part for review in combination with said one of said first part.

Aspect 137: The method per aspect 136 including establishing said assessed quantifiable categories as:

i. basic attitudes/outlooks, core beliefs, behavior and commitment to wellness, plus mental and emotional balance and health, self-love, and relationship(s)' wellness;

ii. Eating and drinking habits, the use of natural foods, low-fat nutrition and dietary habits, World Nutrition Metric System under the Dietary Guidelines for Americans recommended nutrition periodization consumption vs. actual nutrition periodization consumption values;

iii. Regular gentle non-stressful aerobic physical activities and cardiovascular exercise habits;

iv. Alcohol, smoking and drug consumption;

v. environment, health and safety behaviors, and environmental habits;

vi. Use of vitamins/minerals/herbs supplements and pure water;

vii. Personal physical, genetic, medical and immune histories;

viii. Physical, genetic, medical and immune histories;

ix. Emotionally, Healthy (nurturing) relationships;

x. major stress/distress and risk evaluations;

xi. Frequency and quantity of any medications used;

xii. Age;

xiii. Race; and

xiv. Sex

All said categories comprising subjective non-physical assessed parameters of scientific total peak, health, wellness, overweight and obesity fitness, nutrition and any associated health risks.

Aspect 138: The method per aspect 136 including establishing said measured quantifiable categories as:

i. resting heart (pulse) rates;

ii. Exercise heart (pulse) rates;

iii. Recovery heart (pulse) rates;

iv. Oxygen utilization;

v. systolic blood pressure;

vi. Diastolic blood pressure.

vii. Body mass and makeup composition testing (the percentage of fat to muscle);

viii. Circulation efficiency (stress/distress temperature);

ix. Height and weight ratio;

x. type of (body) frame;

xi. Urine analyses;

xii. Saliva (sputum) analyses;

xiii. Blood cells analyses; and

xiv. Vital lung capacity pulmonary function and forced expiratory volume;

All said measured categories comprising objective physiological measurements of scientific total peak, health, wellness, overweight and obesity fitness, nutrition and any associated health risks.

Aspect 139: The method per aspect 136 including establishing said laboratory blood analyzed quantifiable categories as:

i. total blood cholesterol lipids;

ii. Total blood triglyceride lipids;

iii. Total blood LDL lipids;

iv. Total blood HDL lipids;

v. total cholesterol to HDL ratio;

vi. LDL to HDL ratio;

vii. Liver function;

viii. Glucose;

ix. Sugars;

x. proteins;

xi. Vitamins;

xii. Minerals;

xiii. Total immune system natural killer cell (NK) activity;

xiv. Total immune system vitality and NKHT3 percentage;

xv. Total auto immunology NK, B, and T-cell counts; and

xvi. Immune T-cell helper/suppressor ratio;

All said laboratory blood analyzed categories comprising objective laboratory parameters of scientific total peak, health, wellness, overweight and obesity fitness, nutrition and any associated health risks.

Aspect 140: The method per aspect 136 including in method step “e” preparing said evaluation in the form of at least one visually accented graph, each graph having an associated report.

Aspect 141: The method per aspect 140 in which said graph has a graduated 10-point metric scale.

Aspect 142: The method per aspect 141 in which each two points of said 10-point metric scale depict one of said analysis information categories.

Aspect 143: A system for utilizing at least two of a series of assessing, and measuring, and laboratory analyzing scientific sets or groups of pertinent, validated, correlations and predetermined categories, parameters and variables of total peak, health, wellness, overweight and obesity fitness, nutrition and overall associated fitness, nutrition, and health risks of at least one of O'Baymetrics participants and groups, the system comprising:

a. data input means receptive of various testing data from a given person corresponding to specific predetermined sets or groups of pertinent, validated mid correlatable categories, parameters and variables of total peak, health, wellness, overweight and obesity fitness as well as any overall associated fitness, nutrition, and health risks for predicting total peak, health, wellness, overweight and obesity fitness, nutrition and overall associated fitness, nutrition, and health risks;

b. means for transforming the testing data from each pertinent, validated and correlatable category, parameter and variable to produce transformed data for each category, parameter and variable;

c. means storing a data base of transformed data from previous or other total peak, health, wellness, overweight and obesity fitness, nutrition and any overall associated fitness, nutrition, and health risks assessment systems for previously assessed, and measured, and laboratory analyzed O'Baymetrics participants and groups;

d. means for determining the optimal ranges of total peak, health, wellness, overweight and obesity fitness and any overall associated O'Baymetrics and health risks as well as standard mean and standard deviation values from the O'Baymetrics database in accordance with an actual occurrence of the given outcome for previously assessed, and measured, and laboratory analyzed O'Baymetrics participants and groups;

e. means for comparing the transformed data with any pertinent, validated and correlatable categories, parameters and variables of total peak, health, wellness, overweight and obesity fitness, nutrition and overall associated fitness, nutrition, and health risks as well as the standard means and the standard deviation values to assess, and measure, and laboratory analyze the likelihood; of given outcomes for O'Baymetrics participants and groups;

f. means for comparing the transformed data with any pertinent, validated and correlatable optimal ranges as well as the standard mean and standard deviation values to assess, and measure, and laboratory analyze the likelihood of given outcomes for O'Baymetrics participants and groups; and,

g. means for locally updating the data base of said system with the actual occurrence for the given O'Baymetrics participants and groups;

h. wherein the means for determining the optimal ranges of total peak, health, wellness, overweight and obesity fitness, nutrition and overall associated fitness, nutrition, and health risks as well as the standard means and the standard deviation values includes means for locally weighting and adjusting the pre-determined optimal ranges of total peak, health, wellness, overweight and obesity fitness, nutrition and overall associated fitness, nutrition, and health risks as well as the standard means and the standard deviation values of said system for the locally updated data base, whereby the weighted and adjusted means and the standard deviation values for said system are weighted and adjusted for at least one of a particular fitness maintenance metric participants and a group's set of assessed, and measured, and laboratory analyzed parameters by said system relative to the optimal ranges of total peak, health, wellness, overweight and obesity fitness, nutrition and overall associated fitness, nutrition, and health risks as well as the standard means and the standard deviation values determined from the dam base from previous or other total peak, health, wellness, overweight and obesity fitness, nutrition and overall associated medical/risk assessment systems,

i. and including means for preparing an evaluation of said knowledge by correlating and comparing said knowledge with (1) ideal predetermined rankings of total peak, health, wellness, overweight and obesity fitness, nutrition and associated health risks; (2) said scientific total peak, health, wellness, overweight and obesity fitness, nutrition and any associated health risks and fitness maintenance database; and (3) any previously scientific total peak, health, wellness, overweight and obesity fitness, nutrition and any associated health risks assessed and measured, and laboratory analyzed from one fitness maintenance metric participants and groups to create a report having two parts, a first part having a series of visual depictions each having analysis information categorized as:

i. Excellent O'Baymetrics, also known as extremely low risk,

ii. Good O'Baymetrics, also known as low risk,

iii. Average O'Baymetrics, also known as avenge risk,

iv. Poor O'Baymetrics, also known as high risk,

v. Dangerous O'Baymetrics, also known as extremely high risk.

Aspect 144: The system according to aspect 140, further comprising means for storing predetermined requirements for total peak, health, wellness, overweight and obesity fitness and overall associated fitness, nutrition, and health risks data comprising the testing data; all assessed, and measured, and laboratory analyzed sets or groups of pertinent, validated and correlatable categories, parameters and variables of total peak, health, wellness, overweight and obesity fitness and overall associated fitness, nutrition, and health risks; the transformed data; the weighted and adjusted data; the determined standard means; the determined standard deviations; and the means for validating and correlating the data when received, transformed and determined with regard to the predetermined requirements and the means for indicating when the data does not meet the requirements.

Aspect 145: The system per aspect 140, including means for assessing specific sets or groups of pertinent, validated and correlatable categories, parameters and variables of total peak, health, wellness, overweight and obesity fitness and overall associated fitness, nutrition, and health risks, wherein said at least ten categories, parameters and variables are selected from the non-physical categories, parameters and variables consisting of:

i. basic attitudes/outlooks, core beliefs, behaviors and commitment to O'Baymetrics plus mental and emotional balance and health, self-love and relationship(s)' maintenance;

ii. Eating and drinking habits, the use of natural foods, low-fat nutrition and dietary habits;

iii. Regular gentle non-stressful aerobic physical activities and cardiovascular exercise habits;

iv. Alcohol, smoking and drug consumption;

v. environment, health and safety behaviors, and environmental habits;

vi. Use of vitamins/minerals/herbs supplements and pure water;

vii. Personal physical, genetic, medical and immune histories;

viii. physical, genetic, medical and immune histories;

ix. Healthy (nurturing) relationships;

x. major stress/distress and risk evaluations;

xi. Frequency and quantity of any medications used;

xii. Age;

xiii. Race; and

xiv. Sex.

Aspect 146: The system per aspect 140, including means for measuring specific sets or groups of pertinent, validated and correlatable categories, parameters and variables of total peak, health, wellness, overweight and obesity fitness and overall associated fitness maintenance and health risks, wherein said at least ten categories, parameters and variables are selected from the physiological categories, parameters and variables consisting of:

i. resting heart (pulse) rates;

ii. Exercise heart (pulse) rates;

iii. Recovery heart (pulse) rates;

iv. Oxygen utilization;

v. systolic blood pressure;

vi. Diastolic blood pressure;

vii. Body mass and makeup composition measurements (the percentage of fat to muscle);

viii. Circulation efficiency (stress/distress temperature);

ix. Height and weight ratio;

x. type of (body) frame;

xi. Urine analyses;

xii. Saliva (sputum) analyses;

xiii. Blood calls analyses; and,

xiv. Vital lung capacity pulmonary function and forced expiratory volume.

Aspect 147: The system according to aspect 140, including means for laboratory biochemical blood analyzing specific sets or groups of pertinent, validated and correlatable categories, parameters and variables of total peak, health, wellness, overweight and obesity fitness and overall associated fitness maintenance and health risks, wherein said at least twelve categories, parameters and variables are selected from the laboratory biochemical blood analyzed categories, parameters and variables consisting of:

i. total blood cholesterol lipids;

ii. Total blood triglyceride lipids;

iii. Total blood LDL lipids;

iv. Total blood HDL lipids;

v. total cholesterol to HDL ratio;

vi. LDL to HDL ratio;

vii. Liver function;

viii. Glucose;

ix. Sugars;

x. proteins;

xi. Vitamins;

xii. Minerals;

xiii. Total immune system natural killer cell (NK) activity;

xiv. Total immune system vitality and NKHT3 percentage;

xv. Total auto immunology NK, B, and T-cell counts; and

xvi. Immune T-cell helper/suppressor ratio.

Aspect 148: The system according to aspect 140, wherein at least two pertinent, validated and correlatable categories, parameters and variables of total peak, health, wellness, overweight and obesity fitness and overall associated fitness maintenance and health risks are used and further comprising means for correlating the comparisons and correlations of the transformed data for the at least two pertinent, validated and correlatable categories, parameters and variables of total peak, health, wellness, overweight and obesity fitness and overall associated fitness maintenance and health risks.

Aspect 149: A method for administrating a peak performance center program comprising: generating an individualized fitness records for participants being considered for participation in our onsite and mobile peak fitness and/or nutrition periodization training program; create an Individualized Fitness maintenance Plan for each onsite and mobile peak fitness and/or nutrition periodization training participant based upon, but not limited to, VO2 max test, lactate threshold analysis, metabolic rate analysis, swim-stroke force analysis, dry-land strength test, spin scan, motion analysis and body composition measurements criteria, including obtaining qualification data for each of the onsite and mobile peak fitness and/or nutrition periodization training participants, determining which type of services and education counseling, videos are to be provided to each of the onsite and mobile peak fitness and/or nutrition periodization training participants as part of the O'Bay System program based upon the qualification data, and storing the qualification data and an indication of the services to be provided to each mobile peak fitness and/or nutrition periodization training participants in the World Fitness Maintenance Network; and generating forms and reports for implementing an Individualized Fitness maintenance Plan for qualified onsite and mobile peak fitness and/or nutrition periodization training participants, the forms and reports complying with regulatory guidelines for implementing the onsite and mobile peak fitness and/or nutrition periodization training program, and wherein the forms and reports also include a permission form to be signed by a person giving permission for the onsite and mobile peak fitness and/or nutrition periodization training of an onsite and mobile peak fitness and/or nutrition periodization training participant to occur.

Aspect 150: The method for administering an Individualized Fitness Maintenance Plan per aspect 149 further comprising assessing onsite and mobile peak fitness and/or nutrition periodization training participants' progress in an Individual's Fitness Maintenance Plan.

Aspect 151: The method for administering an Individualized Fitness Maintenance Plan per aspect 149, wherein the step of creating an O'Baymetrics Quotient Record (OQR) further comprises setting goals for each onsite and mobile peak fitness and/or nutrition periodization training participant based on the qualification data and storing the goals in the World Fitness Maintenance Network.

Aspect 152: The method for administering an Individualized Fitness Maintenance Plan per aspect 149, wherein the step of creating an O'Baymetrics Quotient Record (OQR) further comprises setting objectives for each onsite and mobile peak fitness and/or nutrition periodization training participant based on the qualification data and storing the objectives in the World Fitness Maintenance Network.

Aspect 153: The method for administering an Individualized Fitness Maintenance Plan per aspect 149, wherein the step of generating forms and reports further comprises creating forms for reviewing onsite and mobile peak fitness and/or nutrition periodization training participants and determining the eligibility of the onsite and mobile peak fitness and/or nutrition periodization training participants.

Aspect 154: The method for administering an Individualized Fitness Maintenance Plan according to aspect 150 wherein the step of generating onsite and mobile peak fitness and/or nutrition periodization training participant information records comprises: generating a unique onsite and mobile peak fitness and/or nutrition periodization training participant identifier for each onsite and mobile peak fitness and/or nutrition periodization training participant being considered for participation in an onsite and mobile Fitness maintenance Plan; inputting information concerning each onsite and mobile peak fitness and/or nutrition periodization periodization training participant being considered for participation in an Individualized Fitness maintenance Plan, including each onsite and mobile peak fitness and/or nutrition periodization training participant's personal data and education counseling, data; and; storing the information in the World Fitness Maintenance Network.

Aspect 155: The method for administering an Individualized Fitness Maintenance Plans per aspect 150 wherein the step of creating an O'Baymetrics Quotient Record (OQR) comprises creating an initial onsite and mobile Fitness Maintenance Record for new onsite and mobile peak fitness maintenance training participants.

Aspect 156: The method for administering an Individualized Fitness Maintenance Plan per aspect 150 wherein the step of creating an O'Baymetrics Quotient Record (OQR) comprises creating an annual O'Baymetrics Quotient Records for monitoring onsite and mobile peak fitness maintenance training participants in a Fitness Maintenance Plan.

Aspect 157: The method for administering an Individualized Fitness Maintenance Plan per aspect 150 wherein the step of creating onsite and mobile peak fitness maintenance training records further comprises creating triennial an onsite and mobile Fitness maintenance records for evaluating onsite and mobile peak fitness and/or nutrition periodization training participant's progress in an Individualized Fitness Maintenance Plan.

Aspect 158: The method for administering an Individualized Fitness Maintenance Plan per aspect 150 wherein the step of assessing onsite and mobile peak fitness and/or nutrition periodization training participant's progress in an Individualized Fitness Maintenance Plans comprises retesting an onsite and mobile peak fitness and/or nutrition periodization training participant to obtain new qualification data.

Aspect 159: The method for administering an Individualized Fitness Maintenance Plan per aspect 150 wherein the step of assessing onsite and mobile peak fitness and/or nutrition periodization training participants' progress in an Individualized Fitness Maintenance Plans comprises verifying onsite and mobile peak fitness and/or nutrition periodization training participant information.

Aspect 160: The method of aspect 149 wherein the Individualized Fitness Maintenance Plan comprises instruction from a one-on-one, group, computer-based or video education counseling, format program.

Aspect 161: The method of aspect 149 wherein the Individualized Fitness Maintenance Plan comprises questions from either a one-on-one, group or video education counseling, format program

Aspect 162: The method of aspect 149 wherein the onsite and mobile peak fitness and/or nutrition periodization training participant's answers to the questions are sent to the central computer to create and update the World Fitness Maintenance Network.

Aspect 163: The method of aspect 149 wherein the onsite and mobile peak fitness and/or nutrition periodization training program comprises an onsite and mobile peak fitness and/or nutrition periodization training participant data view program for displaying a selected subset of the data in graphical form.

Aspect 164: The method of aspect 149 wherein the onsite and mobile peak fitness and/or nutrition periodization training program comprises a document view program for displaying an O'Baymetrics Quotient Record document.

Aspect 165: The method of aspect 149 wherein the education counseling, document is retrieved from wireless or on-line services connected to communicate to the World Fitness Maintenance Network.

Aspect 166: The method of aspect 149 wherein the embedded pointer to the World Fitness Maintenance Network as comprised in an icon.

Aspect 167: A method for administrating the fitness maintenance center program. This triathlon, fitness and/or nutrition program comprising: generating an Individualized Fitness Maintenance Plan (IFP™), Individualized Triathlon Plan (ITP™), and Individualized Nutrition Plan (INP™) and an O'Baymetrics Quotient Record™ (OQR™) for individual medically and weight-challenged fitness participants being considered for participation in the World Fitness Maintenance Center training program; create an Individualized Fitness, Triathlon and Nutrition Plan and an O'Baymetrics Quotient Record™ (OQR™) for each medically and weight-challenged fitness participant based upon predetermined sports medicine and exercise science qualification criteria like a VO2 max test, lactate threshold analysis, metabolic rate analysis, swim-stroke force analysis, dry-land strength test, spin scan, motion analysis and body composition. The fitness maintenance center program also includes obtaining qualification data for each of the medically and weight-challenged fitness participants, determining which type of services and education counseling, videos are to be provided to each of the medically and weight-challenged fitness participants as part of the fitness maintenance center training program based upon the qualification data, and storing the qualification data and an indication of the services to be provided to each medically and weight-challenged fitness participants in the fitness maintenance database; and generating forms and reports for implementing an Individualized Fitness, Triathlon and Nutrition Plan for qualified medically and weight-challenged fitness participants, the forms and reports complying with regulatory guidelines for implementing the fitness maintenance center training program, and wherein the forms and reports also include a permission form to be signed by a person giving permission for an evaluation of a medically and weight-challenged fitness participant to occur.

Aspect 168: The method for administering an Individualized Fitness, Triathlon and Nutrition Plan per aspect 167 further comprising assessing medically and weight-challenged fitness participants' progress in an Individualized Fitness, Triathlon and Nutrition Plan.

Aspect 169: The method for administering an Individualized Fitness, Triathlon and Nutrition Plan per aspect 167, wherein the step of creating an Individualized Fitness, Triathlon and Nutrition Plan and record further comprises setting goals for each medically and weight-challenged fitness participant based on the qualification data and storing the goals in the Fitness maintenance database.

Aspect 170: The method for administering an individualized Fitness, Triathlon and Nutrition Plan per aspect 167, wherein the step of creating an Individualized Fitness, Triathlon and Nutrition Plan and records further comprises setting objectives for each medically and weight-challenged fitness participant based on the qualification data and storing the objectives in the Fitness maintenance database.

Aspect 171: The method for administering an Individualized Fitness, Triathlon and Nutrition Plan per aspect 167, wherein the step of generating forms and reports further comprises creating forms for reviewing training participants and determining the eligibility of fitness individual participants.

Aspect 172: The method for administering an Individualized Fitness, Triathlon and Nutrition Plan according to aspect 168, wherein the step of generating medically and weight-challenged fitness participant individual education records comprises: generating a unique medically and weight-challenged fitness participant identifier for each medically and weight-challenged fitness participant being considered for participation in an Individualized Fitness, Triathlon and Nutrition Plan; inputting individual education concerning each medically and weight-challenged fitness participant being considered for participation in an Individualized Fitness, Triathlon and Nutrition Plan, including each medically and weight-challenged fitness participants personal data and education counseling, data; and; storing the individual education in the Fitness maintenance database.

Aspect 173: The method for administering an Individualized Fitness, Triathlon and Nutrition Plan per aspect 168, wherein the step of creating an Individualized Fitness, Triathlon and Nutrition Plan and records comprises creating an initial and an Individualized Fitness, Triathlon and Nutrition Plan and record for new medically and weight-challenged fitness participants.

Aspect 174: The method for administering an Individualized Fitness, Triathlon and Nutrition Plan per aspect 171, wherein the step of creating an Individualized Fitness, Triathlon and Nutrition Plan and records comprises creating an annual an Individualized Fitness, Triathlon and Nutrition Plan and records for monitoring individual medically and weight-challenged fitness participants in an Individualized Fitness, Triathlon and Nutrition Plan.

Aspect 175: The method for administering an Individualized Fitness, Triathlon and Nutrition Plan per aspect 168, wherein the step of creating an Individualized Fitness, Triathlon and Nutrition Plan and records further comprises creating an annual, Individualized Fitness, Triathlon and Nutrition Plan and records for evaluating medically and weight-challenged fitness participant's progress in an Individualized Fitness, Triathlon and Nutrition Plan.

Aspect 176: The method for administering an Individualized Fitness, Triathlon and Nutrition Plan per aspect 168, wherein the step of assessing medically and weight-challenged fitness participants' progress in an Individualized Fitness, Triathlon and Nutrition Plan comprises retesting a medically and weight-challenged fitness participant to obtain new qualification data.

Aspect 177: The method for administering an Individualized Fitness, Triathlon and Nutrition Plan per aspect 168, wherein the step of assessing the medically and weight-challenged fitness participants' progress in an Individualized Fitness, Triathlon and Nutrition Plan comprises verifying the medically and weight-challenged fitness participant individual education.

Aspect 178: The method of aspect 167, wherein the Individualized Fitness, Triathlon and Nutrition Plan comprise instruction from one-on-one, group, computer-based or video education counseling, format program.

Aspect 179: The method of aspect 167, wherein an Individualized Fitness, Triathlon and Nutrition Plan comprises questions from either a one-on-one, group or video education counseling, format program

Aspect 180: The method of aspect 167, wherein the medically and weight-challenged fitness participants' answers to the questions are sent to the central fitness computer to create and update the Fitness maintenance database.

Aspect 181: The method of aspect 167, wherein the Individualized Fitness, Triathlon and Nutrition Plan and program comprises a medically and weight-challenged fitness participant data view program for displaying a selected subset of the data in graphical form.

Aspect 182: The method of aspect 170, wherein the Individualized Fitness, Triathlon and Nutrition Plan comprises a document view program for displaying the Individualized Fitness, Triathlon and Nutrition Plan and a Fitness maintenance program document.

Aspect 183: The method of aspect 167, wherein the education counseling, document is retrieved from a wireless or on-line Oregon Bay Fitness Library services connected to communicate to the World Fitness Maintenance Network.

Aspect 184: The method of aspect 167, wherein the embedded pointer to the World Fitness Maintenance Library comprises as an icon.

Aspect 185: A method for administrating the Research institution program. This health, fitness and nutrition-based research program comprising: generating an Individualized Triathlon, Fitness maintenance Research Plan and record for a Triathlon, Fitness maintenance Research participants being considered for participation in the Research institution program; create an Individualized Triathlon, Fitness maintenance Research Plan and record for each and every Triathlon, Fitness maintenance Research participant based upon predetermined sports medicine and exercise science qualification criteria like a VO2 max test, lactate threshold analysis, metabolic rate analysis, swim-stroke force analysis, dry-land strength test, spin scan, motion analysis and body composition. The Research institution program also includes obtaining qualification data for each of the individual Triathlon, Fitness maintenance Research participants, determining which type of services and education counseling, videos are to be provided to each of the individual Triathlon, Fitness maintenance Research participants as part of the Health Research institution Program based upon the qualification data, and storing the qualification data and an indication of the services to be provided to each Triathlon, Fitness maintenance Research participants in the Fitness maintenance database; and generating forms and reports for implementing an Individualized Triathlon, Fitness maintenance Research Plan for qualified Triathlon, Fitness maintenance Research participants, the forms and reports complying with regulatory guidelines for implementing the Research institution program, and wherein the forms and reports also include a permission form to be signed by a person giving permission for Triathlon, Fitness maintenance Research participant to occur.

Aspect 186: The method for administering an Individualized Triathlon, Fitness Maintenance Research Plan per aspect 185 further comprising assessing Triathlon, Fitness Maintenance Research participants' progress in the Individualized Triathlon, Fitness Maintenance Research Plans.

Aspect 187: The method for administering an Individualized Triathlon, Fitness Maintenance Research Plan per aspect 185, wherein the step of creating an Individualized Triathlon, Fitness maintenance Research Plan and record further comprises setting goals for each Triathlon, Fitness maintenance Research participant based on the qualification data and storing the goals in the Fitness maintenance database.

Aspect 188: The method for administering an Individualized Triathlon, Fitness maintenance Research Plan per aspect 185, wherein the step of creating an Individualized Triathlon, Fitness maintenance Research Plan and records further comprises setting objectives for each Triathlon, Fitness maintenance Research participant based on the qualification data and storing the objectives in the Fitness maintenance database.

Aspect 189: The method for administering an Individualized Triathlon, Fitness maintenance Research Plan per aspect 185, wherein the step of generating forms and reports further comprises creating forms for reviewing Triathlon, Fitness maintenance Research participants and determining the eligibility of individual Triathlon, Fitness maintenance Research participants.

Aspect 190: The method for administering an Individualized Triathlon, Fitness maintenance Research Plan according to aspect 186, wherein the step of generating a Triathlon, Fitness maintenance Research participant individual education records comprises: generating a unique Triathlon, Fitness maintenance Research participant identifier for each Triathlon, Fitness maintenance Research participant being considered for participation in an Individualized Triathlon, Fitness maintenance Research Plan; inputting individual education concerning each Triathlon, Fitness maintenance Research participant being considered for participation in an Individualized Triathlon, Fitness maintenance Research Plan, including each Triathlon, Fitness maintenance Research participant's personal data and education counseling, data; and; storing the individual education in the Fitness maintenance database.

Aspect 191: The method for administering an Individualized Triathlon, Fitness maintenance Research Plan per aspect 186, wherein the step of creating an Individualized Triathlon, Fitness maintenance Research Plan and records comprises creating an initial Individualized Triathlon, Fitness maintenance Research Plan and record for new Triathlon, Fitness maintenance Research participants.

Aspect 192: The method for administering an Individualized Triathlon, Fitness maintenance Research Plan per aspect 186, wherein the step of creating an Individualized Triathlon, Fitness maintenance Research Plan and records comprises creating an annual, Individualized Triathlon, Fitness maintenance Research Plan and records for monitoring the Triathlon, Fitness maintenance Research participants in an Individualized Triathlon, Fitness maintenance Research Plan.

Aspect 193: The method for administering an Individualized Triathlon, Fitness maintenance Research Plan per aspect 186, wherein the step of creating an Individualized Triathlon, Fitness maintenance Research Plan and records further comprises creating a triennial Individualized Triathlon, Fitness maintenance Research Plan and records for evaluating the Triathlon, Fitness maintenance Research participant's progress in an Individualized Triathlon, Fitness maintenance Research Plan.

Aspect 194: The method for administering Individualized Triathlon, Fitness maintenance Research Plan per aspect 186, wherein the step of assessing individual Triathlon, Fitness maintenance Research participant's progress in an Individualized Triathlon, Fitness maintenance Research Plan comprises retesting a Triathlon, Fitness maintenance Research participant to obtain new qualification data.

Aspect 195: The method for administering an Individualized Triathlon, Fitness maintenance Research Plan per aspect 186, wherein the step of assessing the individual Triathlon, Fitness maintenance Research participants' progress in Individualized Triathlon, Fitness maintenance Research Plans comprises verifying Triathlon, Fitness maintenance Research participant individual education.

Aspect 196: The method of aspect 185, wherein the Individualized Triathlon, Fitness maintenance Research Plans comprises instruction from a one-on-one, group, computer-based or video education counseling, format program.

Aspect 197: The method of aspect 185, wherein an Individualized Triathlon, Fitness maintenance Research Plan comprises questions from either a one-on-one, group or video education counseling, format program

Aspect 198: The method of aspect 185, wherein the Triathlon, Fitness maintenance Research participant's answers to the questions are sent to the central computer to create and update in the Fitness maintenance database.

Aspect 199: The method of aspect 185, wherein the individual of the Oregon Bay Fitness program comprises a Triathlon, Fitness maintenance Research participant data view program for displaying a selected subset of the data in graphical form.

Aspect 200: The method of aspect 185, wherein the individual of the Oregon Bay Fitness program comprises a document view program for displaying in the individualized, fitness plan, in the Oregon Bay Fitness program and the O'Baymetrics Quotient Record document.

Aspect 201: The method of aspect 185, wherein the education counseling, document is retrieved from a wireless or on-line Oregon Bay Fitness services connected to communicate to the World Fitness Maintenance Network.

Aspect 202: The method of aspect 185, wherein the embedded pointer to the World Fitness Maintenance Network comprises as an icon.

Aspect 203: The Triathlon and Nutrition Periodization Training System is a computer-implemented system for training and certifying a triathlon and nutrition periodization trainee to perform triathlon and nutrition periodization task, the system comprising: a data storage for storing preset limit data to determine certified ability of the triathlon and nutrition periodization trainee and for storing log data of the triathlon and nutrition periodization trainee; a controller for receiving input data of the triathlon and nutrition periodization trainee from an input device, sending control data to an output device and sending said log data of the triathlon and nutrition periodization trainee to said data storage; a supervisor for interpreting said input data, determining said control data to be sent by said controller to said output device and determining said input data to be stored as said log data in said data storage, said supervisor further monitoring trainee's interaction with simulations of a portion of said triathlon and nutrition periodization task to determine control data sent to said output device to review instructions and information pertaining to said portion of said triathlon and nutrition periodization task if it is determined that said triathlon and nutrition periodization trainee's interaction with simulations of said portion of said triathlon and nutrition periodization task is not acceptable or to provide a test pertaining to said portion of said triathlon and nutrition periodization task if it is determined that said triathlon and nutrition periodization trainee's interaction with simulations of said portion of said triathlon and nutrition periodization task is acceptable, such that said interaction with said simulations and said review or test pertaining to said portion of said triathlon and nutrition periodization task is thereafter repeated for other portions of said triathlon and nutrition periodization task, and is followed by a post testing of the triathlon and nutrition periodization trainee's overall knowledge of said triathlon and nutrition periodization task for comprising said log data; and a comparator that is accessible to said data storage to compare said log data with said preset limit data to determine certified ability of the triathlon and nutrition periodization trainee.

Aspect 204: The system as set forth in aspect 203 wherein said supervisor includes several software modules.

Aspect 205: A computer method for training and certifying a triathlon and nutrition periodization trainee to perform a triathlon and nutrition periodization task, the method comprising the computer implemented steps of:

a. receiving input data from triathlon and nutrition periodization trainee using a computer-controlled input device;

b. providing to the triathlon and nutrition periodization trainee, through a computer-controlled output device, instructions and information to do a portion of the triathlon and nutrition periodization task;

c. providing to the triathlon and nutrition periodization trainee, through said computer controlled output device, simulations of said portion of said triathlon and nutrition periodization task;

d. monitoring triathlon and nutrition periodization trainee's interaction with said simulations of said portion of said triathlon and nutrition periodization task to determine control data sent to said output device to review instructions and information if said triathlon and nutrition periodization trainee's interaction with simulations of said portion of said triathlon and nutrition periodization task is not acceptable or provide a test related to said portion of said triathlon and nutrition periodization task if said triathlon and nutrition periodization trainee's interaction with simulations of said portion of said triathlon and nutrition periodization task is acceptable;

e. testing the triathlon and nutrition periodization trainee's knowledge of said portion of said triathlon and nutrition periodization task using said output device and said input device;

f. responsive to said testing or simulations, providing on said output device additional information and instructions to the triathlon and nutrition periodization trainee;

g. repeating steps c. through e for the same portion of said triathlon and nutrition periodization task if said triathlon and nutrition periodization trainee's knowledge of said portion of said triathlon and nutrition periodization task is not acceptable or other portions of said triathlon and nutrition periodization task if said triathlon and nutrition periodization trainee's knowledge of said portion of said triathlon and nutrition periodization task is acceptable;

h. post-testing the triathlon and nutrition periodization trainee's overall knowledge of all information and instructions for said triathlon and nutrition periodization task provided to triathlon and nutrition periodization trainee using said output device and said input device; and

i. comparing, in said computer, triathlon and nutrition periodization trainee's post-test scores with a proficiency level to determine certified ability.

Aspect 206: A computer method for training and certifying a triathlon and nutrition periodization trainee to perform a triathlon and nutrition periodization task, the method comprising the computer implemented steps of:

a. identifying triathlon and nutrition periodization trainee;

b. pretesting knowledge of the triathlon and nutrition periodization trainee;

c. storing triathlon and nutrition periodization trainee identification and score of pretest in a data storage;

d. instructing triathlon and nutrition periodization trainee to a portion of necessary information to perform a triathlon and nutrition periodization task;

e. providing on said output device a simulation of a portion of the triathlon and nutrition periodization task to be performed for triathlon and nutrition periodization trainee to interact with;

f. monitoring triathlon and nutrition periodization trainee interaction with said simulation to determine need for review;

g. testing triathlon and nutrition periodization trainee knowledge of information necessary to proceed to next portion of said necessary information;

h. repeating steps c. through f. for remaining portions of necessary information;

i. testing triathlon and nutrition periodization trainee knowledge of all portions of said necessary information to perform triathlon and nutrition periodization task; and

j. comparing triathlon and nutrition periodization trainee test result with an acceptable standard to determine certified ability of the triathlon and nutrition periodization trainee.

Aspect 207: A method for administrating the Triathlon Training Adolescent Program comprising: generating a triathlon and nutrition maintenance participants information records for the adolescent triathlon and nutrition maintenance participants being considered for participation in the Triathlon Training Adolescent Program; create an Individualized Triathlon Plan and record for each adolescent triathlon and nutrition maintenance participant based upon predetermined qualification criteria, including obtaining qualification data for each of the adolescent triathlon and nutrition maintenance participants, determining which type of services and education counseling, videos are to be provided to each of the adolescent triathlon and nutrition maintenance participants as part of the Triathlon Training Adolescent Program based upon the qualification data, and storing the qualification data and an indication of the services to be provided to each the adolescent triathlon and nutrition maintenance participants in the Fitness maintenance database; and generating forms and reports for implementing the Individualized Triathlon Plan for qualified adolescent triathlon and nutrition maintenance participants, the forms and reports complying with regulatory guidelines for implementing the Triathlon Training Adolescent Program, and wherein the forms and reports also include a permission form to be signed by a person giving permission for the Triathlon Training Adolescent Program of an adolescent triathlon and nutrition maintenance participant to occur.

Aspect 208: The method for administering the Individualized Triathlon Plan per aspect 207 further comprising assessing the adolescent triathlon and nutrition maintenance participants' progress in an individual's Triathlon Plan.

Aspect 209: The method for administering the Individualized Triathlon Plans per aspect 207, wherein the step of creating a Triathlon Plan and record further comprises setting goals for each adolescent triathlon and nutrition maintenance participant based on the qualification data and storing the goals in the Fitness maintenance database.

Aspect 210: The method for administering the Individualized Triathlon Plan per aspect 207, wherein the step of creating the Individualized Triathlon Plan and records further comprises setting objectives for each adolescent triathlon and nutrition maintenance participant based on the qualification data and storing the objectives in the Fitness maintenance database.

Aspect 211: The method for administering the Individualized Triathlon Plan per aspect 207, wherein the step of generating forms and reports further comprises creating forms for reviewing the adolescent triathlon and nutrition maintenance participants and determining the eligibility of the adolescent triathlon and nutrition maintenance participants.

Aspect 212: The method for administering the Individualized Triathlon Plans according to aspect 208, wherein the step of generating a triathlon and nutrition maintenance participant information records comprises: generating a unique, triathlon and nutrition maintenance participant identifier for each adolescent triathlon and nutrition maintenance participant being considered for participation in the Individualized Triathlon Plan; inputting information concerning each adolescent triathlon and nutrition maintenance participant being considered for participation in the Individualized Triathlon Plan, including each adolescent triathlon and nutrition maintenance participants personal data and education counseling, data; and; storing the information in the Fitness maintenance database.

Aspect 213: The method for administering the Individualized Triathlon Plans per aspect 208, wherein the step of creating the Individualized Triathlon Plan and records comprises creating an initial the Individualized Triathlon Plan and record for new the adolescent triathlon and nutrition maintenance participants.

Aspect 214: The method for administering the Individualized Triathlon Plans per aspect 208, wherein the step of creating the Individualized Triathlon Plan and records comprises creating an annual the Individualized Triathlon Plan and records for monitoring the adolescent triathlon and nutrition maintenance participants in the Individualized Triathlon Plan.

Aspect 215: The method for administering the Individualized Triathlon Plan per aspect 208, wherein the step of creating the Individualized Triathlon Plan and records further comprises creating triennial the Individualized Triathlon Plan and records for evaluating the adolescent triathlon and nutrition maintenance participants progress in the Individualized Triathlon Plan.

Aspect 216: The method for administering the Individualized Triathlon Plans per aspect 208, wherein the step of assessing the adolescent triathlon and nutrition maintenance participants' progress in the Individualized Triathlon Plans comprises retesting adolescent triathlon and nutrition maintenance participant to obtain new qualification data.

Aspect 217: The method for administering the Individualized Triathlon Plan per aspect 208, wherein the step of assessing the adolescent triathlon and nutrition maintenance participants' progress in the Individualized Triathlon Plan comprises verifying the adolescent triathlon and nutrition maintenance participant information.

Aspect 218: The method of aspect 207, wherein the Individualized Triathlon Plans comprises instruction from either a one-on-one, group, computer-based or video educational format services.

Aspect 219: The method of aspect 207, wherein the Individualized Triathlon Plans comprises questions from either a one-on-one, group or video education counseling, format services

Aspect 220: The method of aspect 207, wherein the adolescent triathlon and nutrition maintenance participants' answers to the questions are sent to the central fitness maintenance computer to create and update in the Fitness maintenance database.

Aspect 221: The method of aspect 207, wherein the Triathlon Training Adolescent Program comprises adolescent triathlon and nutrition maintenance participant data view services for displaying a selected subset of the data in graphical form.

Aspect 222: The method of aspect 207, wherein the Triathlon Training Adolescent Program comprises a document view services for displaying the Triathlon Training Adolescent Program documents.

Aspect 223: The method of aspect 207, wherein the education counseling, document is retrieved from a wireless or on-line Oregon Bay Fitness services connected to communicate to the World Fitness Maintenance Network.

Aspect 224: The method of aspect 207, wherein the embedded pointer to the World Fitness Maintenance Network comprises as an icon.

Aspect 225: A method for administrating the Triathlon Training Individual Program comprising: generating an individualized triathlon and nutrition maintenance training participant information records for an adult being considered for participation in an Individualized Triathlon Plan; create an Individualized Triathlon Plan and record for each triathlon and nutrition maintenance training participant based upon predetermined qualification criteria, including obtaining qualification data for each of the triathletes, determining which type of services and education counseling, videos are to be provided to each of the triathletes as part of the Triathlon Training Individual Program based upon the qualification data, and storing the qualification data and an indication of the services to be provided to each triathletes in training; and generating forms and reports for implementing an individual fitness plan for qualified triathletes, the forms and reports complying with regulatory guidelines for implementing the Triathlon Training Individual program, and wherein the forms and reports also include a permission form to be signed by a person giving permission for an evaluation of a triathlon and nutrition maintenance training participation to occur.

Aspect 226: The method for administering Individualized Triathlon Plans per aspect 225 further comprising assessing the triathletes' progress in an individual's fitness plans.

Aspect 227: The method for administering an Individualized Triathlon Plans per aspect 225, wherein the step of creating an Individualized Triathlon Plan and record further comprises setting goals for each triathlon and nutrition maintenance training participant based on the qualification data and storing the goals in the Fitness maintenance database.

Aspect 228: The method for administering Individualized Triathlon Plan per aspect 225, wherein the step of creating an Individualized Triathlon Plan and records further comprises setting objectives for each triathlon and nutrition maintenance training participant based on the qualification data and storing the objectives in the Fitness maintenance database.

Aspect 229: The method for administering an Individualized Triathlon Plans per aspect 225, wherein the step of generating forms and reports further comprises creating forms for reviewing triathletes and determining the eligibility of individual triathletes.

Aspect 230: The method for administering an Individualized Triathlon Plans according to aspect 226, wherein the step of generating a triathlon and nutrition maintenance training participant information records comprises: generating a unique triathlon and nutrition maintenance training participant identifier for each triathlon and nutrition maintenance training participant being considered for participation in a fitness plan; inputting information concerning each triathlon and nutrition maintenance training participant being considered for participation in an Individualized Triathlon Plan, including each triathletes personal data and education counseling, data; and; storing the information in the Fitness maintenance database.

Aspect 231: The method for administering an Individualized Triathlon Plans per aspect 226, wherein the step of creating an Individualized Triathlon Plan and records comprises creating an initial Individualized Triathlon Plan and record for new triathletes.

Aspect 232: The method for administering an Individualized Triathlon Plans per aspect 226, wherein the step of creating an Individualized Triathlon Plan and records comprises creating an annual, Individualized Triathlon Plan and records for monitoring participants in an Individualized Triathlon Plan.

Aspect 233: The method for administering Individualized Triathlon Plans per aspect 226, wherein the step of creating an Individualized Triathlon Plan and records further comprises creating triennial, Individualized Triathlon Plan and records for evaluating the triathlon's progress in an Individualized Triathlon Plan.

Aspect 234: The method for administering Individualized Triathlon Plans per aspect 226, wherein the step of assessing the triathletes' progress in the Individualized Triathlon Plans comprises retesting a triathlete to obtain new qualification data.

Aspect 235: The method for administering an Individualized Triathlon Plans per aspect 226, wherein the step of assessing a triathletes' progress in an Individualized Triathlon Plans comprises verifying the triathlon and nutrition maintenance training individual information.

Aspect 236: The method of aspect 225, wherein the Individualized Triathlon Plans comprises instruction from either a one-on-one, group, computer-based or video educational format program.

Aspect 237: The method of aspect 225, wherein the Individualized Triathlon Plans comprises questions from either a one-on-one, group or video education counseling, format program

Aspect 238: The method of aspect 225, wherein the triathlete answers to the questions are sent to the central World Fitness Maintenance Network computer to create and update in the Fitness maintenance database.

Aspect 239: The method of aspect 225, wherein the Triathlon Training Individual Program comprises a triathlete data view program for displaying a selected subset of the data in graphical form.

Aspect 240: The method of aspect 225, wherein the Triathlon Training Individual Program comprises a document view program for displaying the Triathlon Training Individual Program documents.

Aspect 241: The method of aspect 240, wherein the education counseling, document is retrieved from a wireless or on-line Oregon Bay Fitness services connected to communicate to the World Fitness Maintenance Network.

Aspect 242: The method of aspect 225, wherein the embedded pointer to the World Fitness Maintenance Network comprises as an icon.

Aspect 243: A method for administrating the cycling fitness maintenance training center program comprising: generating a cycling fitness and/or nutrition periodization participant information records for the cycling fitness and/or nutrition participants being considered for participation in an Individualized Cycling Fitness Maintenance Plan; create an Individualized Cycling Fitness Maintenance Plan and record for each cycling fitness and/or nutrition participant based upon predetermined qualification criteria, including obtaining qualification data for each of cycling fitness maintenance participants, determining which type of services and counseling, videos are to be provided to each of cycling fitness maintenance participants as part of the O'Bay System based upon the qualification data, and storing the qualification data and an indication of the services to be provided to each cycling fitness maintenance participant in the Fitness maintenance database; and generating forms and reports for implementing the Individualized Cycling Fitness Maintenance Plans for qualified cycling fitness maintenance participants, the forms and reports complying with regulatory guidelines for implementing the cycling fitness maintenance training center, and wherein the forms and reports also include a permission form to be signed by a person giving permission for an cycling fitness maintenance participant to occur.

Aspect 244: The method for administering an Individualized Cycling Fitness Maintenance Plans per aspect 243 further comprising assessing cycling fitness maintenance participants' progress in an Individualized Cycling Fitness Maintenance Plans.

Aspect 245: The method for administering an individualized Cycling Fitness Maintenance Plan per aspect 243, wherein the step of creating an Individualized Cycling Fitness Maintenance Plan and record further comprises setting goals for each cycling fitness maintenance participant based on the qualification data and storing the goals in the Fitness maintenance database.

Aspect 246: The method for administering an Individualized Cycling Fitness Maintenance Plan per aspect 243, wherein the step of creating an Individualized Cycling Fitness Maintenance Plan and records further comprises setting objectives for each cycling fitness and/or nutrition participant based on the qualification data and storing the objectives in the Fitness maintenance database.

Aspect 247: The method for administering an Individualized Cycling Fitness Maintenance Plans per aspect 243, wherein the step of generating forms and reports further comprises creating forms for reviewing cycling fitness maintenance participants and determining the eligibility of the cycling fitness maintenance participants.

Aspect 248: The method for administering an Individualized Cycling Fitness Maintenance Plans according to aspect 244, wherein the step of generating cycling fitness and/or nutrition participant information records comprises: generating a unique cycling fitness and/or nutrition participant identifier for each cycling fitness and/or nutrition periodization participant being considered for participation in an Individualized Cycling Fitness Maintenance Plan; inputting information concerning each cycling fitness and/or nutrition periodization participant being considered for participation in an Individualized Cycling Fitness Maintenance Plan, including each participant's personal data and counseling, data; and; storing the information in the Fitness maintenance database.

Aspect 249: The method for administering an Individualized Cycling Fitness Maintenance Plans per aspect 244, wherein the step of creating an Individualized Cycling Fitness Maintenance Plan and records comprises creating an initial O'Baymetrics Quotient Record for new cycling fitness maintenance participants.

Aspect 250: The method for administering an Individualized Cycling Fitness Maintenance Plans per aspect 244, wherein the step of creating O'Baymetrics Quotient Records comprises creating an annual O'Baymetrics Quotient Records for monitoring the cycling fitness maintenance participants in an individualized Cycling Fitness Maintenance Plan.

Aspect 251: The method for administering an Individualized Cycling Fitness Maintenance Plans per aspect 244, wherein the step of creating O'Baymetrics Quotient Records further comprises creating triennial O'Baymetrics Quotient Records for evaluating participant's progress in an Individualized Cycling Fitness Maintenance Plan.

Aspect 252: The method for administering an Individualized Cycling Fitness Maintenance Plans per aspect 244, wherein the step of assessing participant's progress in an Individualized Cycling Fitness Maintenance Plans comprises retesting a cycling fitness and/or nutrition periodization participant to obtain new qualification data.

Aspect 253: The method for administering an Individualized Cycling Fitness Maintenance Plans per aspect 244, wherein the step of assessing the cycling fitness maintenance participants' progress in an Individualized Cycling Fitness Maintenance Plans comprises verifying the cycling fitness and/or nutrition periodization participant information.

Aspect 254: The method of aspect 243, wherein an Individualized Cycling Fitness Maintenance Plans comprises instruction from either a one-on-one, group, computer-based or video format program.

Aspect 255: The method of aspect 243, wherein an Individualized Cycling Fitness Maintenance Plans comprises questions from either a one-on-one, group or video counseling, format program

Aspect 256: The method of aspect 243, wherein the participant's answers to the questions are sent to the central World Fitness Maintenance Network computer to create and update the Fitness maintenance database.

Aspect 257: The method of aspect 243, wherein the cycling fitness maintenance training center comprises a cycling fitness and/or nutrition periodization participant data view program for displaying a selected subset of the data in graphical form.

Aspect 258: The method of aspect 243, wherein the cycling fitness maintenance training center comprises a document view program for displaying O'Bay System documents.

Aspect 259: The method of aspect 258, wherein the counseling, document is retrieved from a wireless or on-line Oregon Bay Fitness service connected to communicate to the World Fitness Maintenance Network.

Aspect 260: The method of aspect 243, wherein the embedded pointer to the World Fitness Maintenance Network comprises as an icon.

Aspect 261: The World Fitness Maintenance Network™ is an online, fitness and/or nutrition information and record searching system for determining a single web site associated with fitness and/or nutrition information and record, comprising: a server having access to the Fitness maintenance database, said fitness maintenance database includes a list of fitness and/or nutrition information, records and products uniquely associated with said fitness and/or nutrition information and record participants, and web sites, said Fitness maintenance database identifies a single web site for each unique combination stored in the Fitness maintenance database of a fitness and/or nutrition information, record and an associated product; at least one exercise physiologist and dietician/nutritionist or certified personal trainer terminal coupled to said server and establishing a communication session with said server through a fitness and/or nutrition information and record communication network, at least one exercise physiologist and dietician/nutritionist or certified personal trainer using said exercise physiologist and dietician/nutritionist or certified personal trainer terminal to enter search criteria, said search criteria comprising both said fitness and/or nutrition information and record and a product associated with said fitness and/or nutrition information and record participant; and said server, in response to receipt of said search criteria, uniquely identifying the single web site corresponding to said search criteria by examining said fitness maintenance database for the unique combination stored in the fitness maintenance database.

Aspect 262: The system per aspect 261, further comprising search system software, wherein said search system software causes said exercise physiologist and dietician/nutritionist or certified personal trainer terminal to display said single web site.

Aspect 263: The system per aspect 261, wherein said web site is identified by a uniform resource locator.

Aspect 264: The system per aspect 261, wherein said web site is identified by a file location for a constructed web site.

Aspect 265: The system according to aspect 261, wherein said fitness maintenance database further comprises a macro-list, said macro-list having a product type and one or more corresponding product entries, wherein the Fitness maintenance database record in said Fitness maintenance database matches said entered search criteria when said entered product associated with said fitness and/or nutrition information and record includes an adolescent entry from said entries corresponding to said product type.

Aspect 266: The system per aspect 261, wherein said Fitness maintenance database further includes at least one of a URL and an actual list of authorized vendors of said product associated with at least one fitness and/or nutrition information and record participant in said list of fitness and/or nutrition information and record, said server further identifying at least one of said URL and an actual list of authorized vendors corresponding to said search criteria.

Aspect 267: A fitness and/or nutrition information and record search engine server in communication with at least one exercise physiologist and dietician/nutritionist or certified personal trainer terminal across a fitness and/or nutrition information and record communication network, said search engine server determining a single web site associated with fitness and/or nutrition information and record, the server comprising: a storage device storing the Fitness maintenance database and search engine software, said Fitness maintenance database comprising a list of the fitness and/or nutrition information and record, products uniquely associated with said fitness and/or nutrition information and record, and web sites, said Fitness maintenance database including a single web site for each unique combination stored in the Fitness maintenance database of the fitness and/or nutrition information and record and an associated product; a network interface, said network interface communicating with said at least one exercise physiologist and dietician/nutritionist or certified personal trainer terminal across said fitness and/or nutrition information and record communication network; a central processing unit executing said search engine software, said search engine software performing the functions of: receiving search criteria from said at least one exercise physiologist and dietician/nutritionist or certified personal trainer terminal, said search criteria comprising both fitness and/or nutrition information, record and products associated with said fitness and/or nutrition information and record; and uniquely identifying said single web site corresponding to said received search criteria by examining said Fitness maintenance database to obtain the unique combination stored in the Fitness maintenance database.

Aspect 268: The server per aspect 261, wherein said Fitness maintenance database further includes at least one of a URL and an actual list of authorized vendors of said product associated with at least one fitness and/or nutrition information and record in said list of fitness and nutrition, said server further identifying at least one of said URL and an actual list of authorized vendors corresponding to said search criteria.

Aspect 269: The server per aspect 261, wherein said search engine software causes said exercise physiologist and dietician/nutritionist or certified personal trainer terminal to display said single web site.

Aspect 270: The server per aspect 261, wherein said web site is identified by a uniform resource locator.

Aspect 271: The server per aspect 261, wherein said web site is identified by a file location for a constructed web site.

Aspect 272: The server according to aspect 261, wherein said search engine software when executed by said central processing unit further performs the functions of: allowing a fitness and/or nutrition information and record participant to use one of said exercise physiologist and dietician/nutritionist or certified personal trainer terminals to register said fitness and/or nutrition information, record and said product associated with said fitness and/or nutrition information and record; verifying that a combination of said fitness and/or nutrition information and record and said product associated with said fitness and/or nutrition information and record are unique within said Fitness maintenance database; and storing said fitness and/or nutrition information, record and said product associated with said fitness and/or nutrition information and record in said Fitness maintenance database in the case where said combination is unique.

Aspect 273: The server per aspect 266, wherein said search engine software when executed by said central processing unit further performs the function of storing at least of a URL and an actual list of authorized vendors of said product associated with said fitness and/or nutrition information and record in one of said Fitness maintenance database and a separate Fitness maintenance database.

Aspect 274: The server per aspect 261, wherein said Fitness maintenance database further includes a registrant field, said registrant field corresponding to an entity associated with fitness and/or nutrition information and record in said Fitness maintenance database.

Aspect 275: A method for determining a single web site associated with fitness and/or nutrition information and record, said method comprising: receiving search criteria from the fitness and/or nutrition information and record personnel terminal, said search criteria comprising a fitness and/or nutrition information and record and a product associated with said fitness and nutrition; and uniquely identifying a single web site corresponding to said search criteria by examining the Fitness maintenance database, said Fitness maintenance database includes a list of fitness and/or nutrition information and record, products uniquely associated with the fitness and/or nutrition information and record, and web sites, and which includes a single web site for each unique combination stored in the Fitness maintenance database of a fitness and/or nutrition information and record and an associated product.

Aspect 276: The method per aspect 275, further comprising the step of causing said exercise physiologist and dietician/nutritionist or certified personal trainer terminal to display said single web site.

Aspect 277: The method per aspect 275, wherein said web site is identified by a uniform resource locator.

Aspect 278: The method per aspect 275, wherein said web site is identified by a file location for a constructed web site.

Aspect 279: The method per aspect 275, further including the step of identifying at least one of a URL and an actual list of authorized vendors of said product associated with said fitness and/or nutrition information and record.

Aspect 280: A method for administrating the Triathlon and Nutrition Periodization Training Program comprising: generating an individualized triathlon and nutrition periodization information records for an adolescent or adult triathlon and nutrition periodization participants being considered for participation in an individualized Triathlon and Nutrition Periodization Training Plan; create an individualized Triathlon and Nutrition Periodization Training Plan and record for each adolescent or adult triathlon and nutrition periodization participant based upon predetermined qualification criteria, including obtaining qualification data for each of the overweight or obese adolescent or adult triathlon and nutrition periodization participants, determining which type of services and education counseling, videos are to be provided to each of triathlon and nutrition periodization participants as part of the individualized Triathlon and Nutrition Periodization Training Program based upon the qualification data, and storing the qualification data and an indication of the services to be provided to each triathlon and nutrition periodization participants in the Fitness maintenance database; and generating forms and reports for implementing an individualized Triathlon and Nutrition Periodization Training Plan for qualified triathlon and nutrition periodization participants, the forms and reports complying with regulatory guidelines for implementing the Triathlon and Nutrition Periodization Training Program, and wherein the forms and reports also include a permission form to be signed by a person giving permission for an evaluation of an Individualized Triathlon participation to occur.

Aspect 281: The method for administering an individualized Triathlon and Nutrition Periodization Training Plans per aspect 280 further comprising assessing the overweight or obese adolescent or adult triathlon and nutrition periodization participants' progress in Triathlon and Nutrition Periodization Training Plans.

Aspect 282: The method for administering an individualized Triathlon and Nutrition Periodization Training Plans per aspect 280, wherein the step of creating an individualized Triathlon and Nutrition Periodization Training Plan and record further comprises setting goals for each overweight or obese adolescent or adult triathlon and nutrition periodization participant based on the qualification data and storing the goals in the Fitness maintenance database.

Aspect 283: The method for administering an individualized Triathlon and Nutrition Periodization Training Plan per aspect 280, wherein the step of creating an individualized Triathlon and Nutrition Periodization Training Plan and records further comprises setting objectives for each overweight or obese adolescent or adult triathlon and nutrition periodization participant based on the qualification data and storing the objectives in the Fitness maintenance database.

Aspect 284: The method for administering an individualized Triathlon and Nutrition Periodization Training Plans per aspect 280, wherein the step of generating forms and reports further comprises creating forms for reviewing the overweight or obese adolescent or adult triathlon and nutrition periodization participants and determining the eligibility of triathlon and nutrition periodization participants.

Aspect 285: The method for administering an individualized Triathlon and Nutrition Periodization Training Plans according to aspect 281, wherein the step of generating an individualized triathlon and nutrition periodization participant information records comprises: generating a unique individualized triathlon and nutrition periodization participant identifier for each overweight or obese adolescent or adult triathlon and nutrition periodization participant being considered for participation in an individualized Triathlon and Nutrition Periodization Training Plan; inputting information concerning each overweight or obese adolescent or adult triathlon and nutrition periodization participant being considered for participation in an individualized Triathlon and Nutrition Periodization Training Plan, including each individualized triathlon and nutrition periodization participant's personal data and education counseling, data; and; storing the information in the Fitness maintenance database.

Aspect 286: The method for administering an individualized Triathlon and Nutrition Periodization Training Plans per aspect 281, wherein the step of creating an individualized Triathlon and Nutrition Periodization Training Plan and records comprises creating an initial, individualized Triathlon and Nutrition Periodization Training Plan and record for the new overweight or obese adolescent or adult triathlon and nutrition periodization participants.

Aspect 287: The method for administering an individualized Triathlon and Nutrition Periodization Training Plans per aspect 281, wherein the step of creating an individualized Triathlon and Nutrition Periodization Training Plan and records comprises creating an annual individualized Triathlon and Nutrition Periodization Training Plan and records for monitoring triathlon and nutrition periodization participants in an individualized Triathlon and Nutrition Periodization Training Plan.

Aspect 288: The method for administering an individualized Triathlon and Nutrition Periodization Training Plans according to aspect 281, wherein the step of creating an individualized Triathlon and Nutrition Periodization Training Plan and records further comprises creating triennial Individualized Triathlon and Nutrition Periodization Training Plan and records for evaluating the overweight or obese adolescent or adult triathlon and nutrition periodization participant's progress in an individualized Triathlon and Nutrition Periodization Training Plan.

Aspect 289: The method for administering an individualized Triathlon and Nutrition Periodization Training Plans per aspect 281, wherein the step of assessing the overweight or obese adolescent or adult triathlon and nutrition periodization participant's progress in an individualized Triathlon and Nutrition Periodization Training Plans comprises retesting the overweight or obese adolescent or adult triathlon and nutrition periodization participant to obtain new qualification data.

Aspect 290: The method for administering an individualized Triathlon and Nutrition Periodization Training Plans per aspect 281, wherein the step of assessing the overweight or obese adolescent or adult triathlon and nutrition periodization participants' progress in an individualized Triathlon and Nutrition Periodization Training Plans comprises verifying Individualized Triathlon, Fitness and/or nutrition periodization Information.

Aspect 291: The method of aspect 280, wherein the individualized Triathlon and Nutrition Periodization Training Plans comprises instruction from either a one-on-one, group, computer-based or video educational format program.

Aspect 292: The method of aspect 280, wherein the individualized Triathlon and Nutrition Periodization Training Plans comprises questions from either a one-on-one, group or video education counseling, format program

Aspect 293: The method of aspect 280, wherein the individual overweight or obese adolescent or adult triathlon and nutrition periodization participant's answers to the questions are sent to the central fitness computer to create and update the Fitness maintenance database.

Aspect 294: The method of aspect 280, wherein the Triathlon and Nutrition Periodization Training Program comprises an individualized overweight or obese adolescent or adult triathlon and nutrition periodization participant data view program for displaying a selected subset of the data in graphical form.

Aspect 295: The method of aspect 280, wherein the Triathlon and Nutrition Periodization Training Program comprises a document view program for displaying Triathlon and Nutrition Periodization Training Program documents.

Aspect 296: The method of aspect 295, wherein the education counseling, document is retrieved from a wireless or on-line Oregon Bay Fitness services connected to communicate to the World Fitness Maintenance Network.

Aspect 297: The method of aspect 280, wherein the embedded pointer to the World Fitness Maintenance Network comprises as an icon.

Aspect 298: A method for administrating an on-demand fitness and/or nutrition distribution system comprising: a customer's electronic data processing device on which an on-demand fitness and/or nutrition application is loaded, the customer's electronic data processing device being configured to execute the on-demand fitness and/or nutrition application to perform operations including: storing a local on-demand fitness and/or nutrition database containing a portion or all of the on-demand fitness and/or nutrition items (i.e. sports medicine physician, exercise science physiologist, dietician, nutritionist, certified personal trainer, cycling wind tunnel specialist and other fitness and/or nutrition service providers) stored in an on-demand fitness and/or nutrition database hosted at a secure server, selecting an on-demand fitness and/or nutrition item stored in the local on-demand fitness maintenance database, displaying the selected on-demand fitness and/or nutrition item or causing the selected on-demand fitness and/or nutrition item to be displayed by a viewer application executing on the customer's electronic data processing device, constructing an email including a hyperlink for downloading or streaming the selected on-demand fitness and/or nutrition item from the on-demand fitness maintenance database of the secure server, and sending the constructed email to a recipient or causing the constructed email to be sent to the recipient by an email application executing on the customer's electronic data processing device.

Aspect 299: The on-demand fitness maintenance distribution system of aspect 298 wherein the customer's electronic data processing device is a mobile device or a notebook or desktop computer.

Aspect 300: The on-demand fitness maintenance distribution system of aspect 298 wherein the customer's electronic data processing device is a tablet computer.

Aspect 301: The on-demand fitness maintenance distribution system of aspect 298 further comprising: said secure server hosting said on-demand fitness maintenance database; wherein the secure server is configured to transmit or stream the selected on-demand fitness and/or nutrition item to a recipient's electronic data processing device responsive to selection of the hyperlink at the recipient's electronic data processing device.

Aspect 302: The on-demand fitness maintenance distribution system of aspect 301 wherein the on-demand fitness and/or nutrition application is not loaded onto the recipient's electronic data processing device.

Aspect 303: The on-demand fitness maintenance distribution system of aspect 301 wherein the secure server is further configured to keep a log of downloads or streams of the on-demand fitness and/or nutrition items.

Aspect 304: The on-demand fitness maintenance distribution system of aspect 301 wherein the secure server is further configured to present a visual representation of statistics of the logged downloads or streams of the on-demand fitness and/or nutrition items.

Aspect 305: The on-demand fitness maintenance distribution system of aspect 301 wherein the secure server is further configured to perform a security check on a URL address associated with the recipient's electronic data processing device and to transmit or stream the selected on-demand fitness and/or nutrition item only if the URL address passes the security check.

Aspect 306: The on-demand fitness maintenance distribution system of aspect 305 wherein the security check including one or more of (1) comparing the URL address with a do-not-send list, (2) comparing the URL address with a permitted URL list, and (3) limiting the number of transmit or stream events to the URL address in a predetermined time interval.

Aspect 307: The on-demand fitness maintenance distribution system of aspect 304 wherein the storing comprises downloading the portion or all the on-demand fitness and/or nutrition items stored in the on-demand fitness maintenance database from the secure server to the customer's electronic data processing device via the internet.

Aspect 308: The on-demand fitness maintenance distribution system of aspect 301 wherein: the customer's electronic data processing device is configured to connect with the internet and the sending operation is performed via the internet, and the customer's electronic data processing device is configured to perform the selecting, displaying, and constructing operations without an internet connection and to delay the sending operation until the customer's electronic data processing device relates to the internet.

Aspect 309: The on-demand fitness maintenance distribution system of aspect 301 wherein: the customer's electronic data processing device is configured to connect with the internet and the sending operation is performed via the internet, and the customer's electronic data processing device is configured to perform the selecting, displaying, and constructing operations using the local on-demand fitness maintenance database without an internet connection.

Aspect 310: A non-transitory storage medium storing instructions executable by an electronic data processing device to perform a method including: selecting an on-demand fitness maintenance(on-demand fitness and/or nutrition) item stored in a local on-demand fitness maintenance database containing content downloaded from an on-demand fitness maintenance database hosted at a secure server; displaying the selected on-demand fitness and/or nutrition item or causing the selected on-demand fitness and/or nutrition item to be displayed by a viewer application executing on the electronic data processing device; constructing an email including a hyperlink for downloading or streaming the selected on-demand fitness and/or nutrition item from the on-demand fitness maintenance database of the secure server; and sending the constructed email to a recipient or causing the constructed email to be sent to the recipient by an email application executing on the electronic data processing device.

Aspect 311: The non-transitory storage medium of aspect 310 wherein: the sending operation utilizes the internet, and the selecting, displaying, and constructing operations are performed using the local on-demand fitness maintenance database and do not utilize the internet.

Aspect 312: The non-transitory storage medium of aspect 310 wherein the method further comprises delaying the sending until an internet connection is available.

Aspect 313: The non-transitory storage medium of aspect 310 wherein the method further comprises downloading the local on-demand fitness maintenance database from a remote server to the electronic data processing device via the internet.

Aspect 314: An on-demand fitness maintenance (on-demand fitness and/or nutrition) distribution system comprising: a server connected with the internet and hosting an on-demand fitness maintenance database storing on-demand fitness and/or nutrition items, the server configured to: connect with authenticated mobile electronic data processing devices via the internet and download portions or all of the on-demand fitness and/or nutrition items stored in the on-demand fitness maintenance database to the authenticated mobile electronic data processing devices via the internet connection; and transmit or stream an on-demand fitness and/or nutrition item to a recipient electronic data processing device via the internet responsive to receipt of a request for the on-demand fitness and/or nutrition item by selection of a hyperlink at the recipient's electronic data processing device identifying the on-demand fitness and/or nutrition item.

Aspect 315: The on-demand fitness maintenance distribution system of aspect 314 wherein the transmit or stream operation includes logging the transmit or stream event including an identification of the recipient at the server.

Aspect 316: The on-demand fitness maintenance distribution system of aspect 314 wherein the transmit or stream operation includes performing a security check on a URL address associated with the hyperlink and transmitting or streaming the selected on-demand fitness and/or nutrition item only if the URL address passes the security check.

Aspect 317: The method of aspect 314, wherein the embedded pointer to the World Fitness Maintenance Network comprises as an icon.

IV. Platform Architecture

The platform 700 may be embodied as, for example, but not be limited to, a website, a web application, a desktop application, and a mobile application compatible with a computing device. The computing device may comprise, but not be limited to, a desktop computer, laptop, a tablet, or mobile telecommunications device. Moreover, the platform 100 may be hosted on a centralized server, such as, for example, a cloud computing service. Although methods 400, 500 and 600 have been described to be performed by a computing device 700, it should be understood that, in some embodiments, different operations may be performed by different networked elements in operative communication with computing device 700.

Embodiments of the present disclosure may comprise a system having a memory storage and a processing unit. The processing unit coupled to the memory storage, wherein the processing unit is configured to perform the stages of methods 400, 500 and 600.

FIG. 7 is a block diagram of a system including computing device 700. Consistent with an embodiment of the disclosure, the aforementioned memory storage and processing unit may be implemented in a computing device, such as computing device 700 of FIG. 7. Any suitable combination of hardware, software, or firmware may be used to implement the memory storage and processing unit. For example, the memory storage and processing unit may be implemented with computing device 700 or any of other computing devices 718, in combination with computing device 700. The aforementioned system, device, and processors are examples and other systems, devices, and processors may comprise the aforementioned memory storage and processing unit, consistent with embodiments of the disclosure.

With reference to FIG. 7, a system consistent with an embodiment of the disclosure may include a computing device or cloud service, such as computing device 700. In a basic configuration, computing device 700 may include at least one processing unit 702 and a system memory 704. Depending on the configuration and type of computing device, system memory 704 may comprise, but is not limited to, volatile (e.g. random access memory (RAM)), non-volatile (e.g. read-only memory (ROM)), flash memory, or any combination. System memory 704 may include operating system 705, one or more programming modules 706, and may include a program data 707. Operating system 705, for example, may be suitable for controlling computing device 700's operation. In one embodiment, programming modules 706 may include image encoding module, machine learning module and image classifying module. Furthermore, embodiments of the disclosure may be practiced in conjunction with a graphics library, other operating systems, or any other application program and is not limited to any particular application or system. This basic configuration is illustrated in FIG. 7 by those components within a dashed line 708.

Computing device 700 may have additional features or functionality. For example, computing device 700 may also include additional data storage devices (removable and/or non-removable) such as, for example, magnetic disks, optical disks, or tape. Such additional storage is illustrated in FIG. 7 by a removable storage 709 and a non-removable storage 710. Computer storage media may include volatile and nonvolatile, removable and non-removable media implemented in any method or technology for storage of information, such as computer readable instructions, data structures, program modules, or other data. System memory 704, removable storage 709, and non-removable storage 710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 700. Any such computer storage media may be part of device 700. Computing device 700 may also have input device(s) 712 such as a keyboard, a mouse, a pen, a sound input device, a touch input device, etc. Output device(s) 714 such as a display, speakers, a printer, etc. may also be included. The aforementioned devices are examples and others may be used.

Computing device 700 may also contain a communication connection 716 that may allow device 700 to communicate with other computing devices 718, such as over a network in a distributed computing environment, for example, an intranet or the Internet. Communication connection 716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 704, including operating system 705. While executing on processing unit 702, programming modules 706 (e.g., application 720) may perform processes including, for example, stages of one or more of methods 500 and 600 as described above. The aforementioned process is an example, and processing unit 702 may perform other processes. Other programming modules that may be used in accordance with embodiments of the present disclosure may include image encoding applications, machine learning application, image classifiers etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

All rights including copyrights in the code included herein are vested in and the property of the Applicant. The Applicant retains and reserves all rights in the code included herein, and grants permission to reproduce the material only in connection with reproduction of the granted patent and for no other purpose.

V. Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A computer implemented method of facilitating operation of a health maintenance device, the computer implemented method comprising:
receiving, using a communication device, a first plurality of health maintenance participant information associated with a health maintenance user, wherein the first plurality of health maintenance participant information comprises a selection of at least one of an interactive learned program mode and an automated health maintenance test mode;
receiving, using a communication device, a second plurality of health maintenance participant information generated based on monitoring each of the health maintenance device and the health maintenance user;
generating, using a processing device, a plurality of performance assessments of the health maintenance user corresponding to a plurality of health maintenance sessions of the health maintenance device, wherein each performance assessment is generated based on the second plurality of the health maintenance participant information and the first plurality of health maintenance participant information;
storing, using a storage device, the plurality of performance assessments;
generating, using the processing device, at least one control code based on a comparing of the second plurality of health maintenance participant information with the health maintenance database; and
transmitting, using the communication device, the at least one control code to the health maintenance device, wherein the health maintenance device is configured to be adjusted based on the at least one control code.

2. The computer implemented method of claim 1, wherein the first plurality of health maintenance information comprises one or more of user data, at least one goal and a selection of an operation mode of the health maintenance device.

3. The computer implemented method of claim 2, wherein a performance assessment of the plurality of performance assessments comprises an energy expenditure associated with the at least one goal.

4. The computer implemented method of claim 3, wherein the performance assessment further comprises a percentage of reaching the at least one goal, wherein the percentage is determined based on dividing the energy expenditure by at least one of a weight and a time length of use of the health maintenance device.

5. The computer implemented method of claim 4 further comprising:
generating, using the processing device, a suggested goal based on a comparison of the percentage with a predetermined low; and
transmitting, using the communication device, the suggested goal to the health maintenance device.

6. The computer implemented method of claim 1, wherein the at least one control code is configured to adjust at least one of a difficulty level and an environmental factor associated with the health maintenance device.

7. The computer implemented method of claim 1, wherein generating the at least one control code is further based on the first plurality of health maintenance participant information.

8. The computer implemented method of claim 1, wherein the second plurality of health maintenance participant information comprises at least one of a weight, a speed and a distance traveled associated with the health maintenance device, wherein the health maintenance device comprises at least one sensor configured to detect at least one of the weight, the speed and the distance travelled.

9. The computer implemented method of claim 2 further comprising:
generating, using the processing device, at least one predetermined health maintenance boundary associated with the health maintenance device, wherein the at least one predetermined health maintenance boundary is generated based on the user data; and
transmitting, using the communication device, the at least one predetermined health maintenance boundary to the health maintenance device, wherein the health maintenance device is configured to generate an alert based on an operational value of the health maintenance device exceeding the at least one predetermined health maintenance boundary.

10. The computer implemented method of claim 9, wherein the user data comprises a physical condition, wherein the predetermined health maintenance boundary is further associated with physiological data of the health maintenance user, wherein the health maintenance device is further configured to detect physiological data of the health maintenance user.

11. A system for facilitating operation of a health maintenance device, the system comprising:
a communication device configured to:
receive a first plurality of health maintenance participant information associated with a health maintenance user, wherein the first plurality of health maintenance participant information comprises a selection of at least one of an interactive learned program mode and an automated health maintenance test mode;
receive a second plurality of health maintenance participant information generated based on monitoring each of the health maintenance device and the health maintenance user;
transmit at least one control code to the health maintenance device, wherein the health maintenance device is configured to be adjusted based on the at least one control code;
a processing device configured to:
generate a plurality of performance assessments of the health maintenance user corresponding to a plurality of health maintenance sessions of the health maintenance device, wherein each performance assessment is generated based on the second plurality of the health maintenance participant information and the first plurality of health maintenance participant information;
generate the at least one control code based on a comparing of the second plurality of health maintenance participant information with the health maintenance database; and
a storage device configured to store the plurality of performance assessments.

12. The system of claim 11, wherein the first plurality of health maintenance information comprises one or more of user data, at least one goal and a selection of an operation mode of the health maintenance device.

13. The system of claim 12, wherein a performance assessment of the plurality of performance assessments comprises an energy expenditure associated with the at least one goal.

14. The system of claim 13, wherein the performance assessment further comprises a percentage of reaching the at least one goal, wherein the percentage is determined based on dividing the energy expenditure by at least one of a weight and a time length of use of the health maintenance device.

15. The system of claim 14, wherein the processing device is further configured to generate a suggested goal based on a comparison of the percentage with a predetermined low, wherein the communication device is further configured to transmit the suggested goal to the health maintenance device.

16. The system of claim 11, wherein the at least one control code is configured to adjust at least one of a difficulty level and an environmental factor associated with the health maintenance device.

17. The system of claim 11, wherein generating the at least one control code is further based on the first plurality of health maintenance participant information.

18. The system of claim 11, wherein the second plurality of health maintenance participant information comprises at least one of a weight, a speed and a distance traveled associated with the health maintenance device, wherein the health maintenance device comprises at least one sensor configured to detect at least one of the weight, the speed and the distance travelled.

19. The system of claim 12, wherein the processing device is further configured to generate at least one predetermined health maintenance boundary associated with the health maintenance device, wherein the at least one predetermined health maintenance boundary is generated based on the user data, wherein the communication device is further configured to transmit the at least one predetermined health maintenance boundary to the health maintenance device, wherein the health maintenance device is configured to generate an alert based on an operational value of the health maintenance device exceeding the at least one predetermined health maintenance boundary.

20. The system of claim 19, wherein the user data comprises a physical condition, wherein the predetermined health maintenance boundary is further associated with physiological data of the health maintenance user, wherein the health maintenance device is further configured to detect physiological data of the health maintenance user.
